(19) United States
(12) Patent Application Publication
(10) Pub. No.: US 2026/0034229 A1
(43) Pub. Date: Feb. 5, 2026

(54) ANTIBODY-TARGETED ENZYMATIC CHEMOPROTECTION

(71) Applicant: THE UNIVERSITY OF CHICAGO, Chicago, IL (US)

(72) Inventors: Savas TAY, Burr Ridge, IL (US); Jonathan MATTHEWS, Chicago, IL (US); Betul CELIKER, Chicago, IL (US)

(21) Appl. No.: 19/361,810

(22) Filed: Oct. 17, 2025

Related U.S. Application Data
(63) Continuation-in-part of application No. PCT/US2024/025502, filed on Apr. 19, 2024.
(60) Provisional application No. 63/497,104, filed on Apr. 19, 2023.

(51) Int. Cl. A61K 47/68; A61K 38/50; A61P 39/00; C07K 16/28

(52) U.S. Cl. CPC A61K 47/6815; A61K 38/50; A61K 47/6849; A61K 47/6889; A61P 39/00; C07K 16/2803; C07K 16/2896; C12Y 305/04005

(57) ABSTRACT

Aspects of the disclosure relate to compositions and methods for reducing toxicity of a cytotoxic agent comprising administering an antigen-binding protein conjugated to a protection molecule.

This application is a continuation in-part of International Application No. PCT/US2024/025502, filed Apr. 19, 2024, which claims priority of U.S. Provisional Application No. 63/467,104 filed Apr. 19, 2023, which is hereby incorporated by reference in its entirety.

This invention was made with government support under HD007009 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Oct. 16, 2025, is named ARCDP0801USCIP.xml and is 1,544,632 bytes in size.

### BACKGROUND OF THE INVENTION

### 1. Field of the Invention

This invention relates to the field of molecular biology and medicine.

### 2. Background

Chemotherapy is an approach to cancer treatment that kills or blocks the growth of malignant cells with drugs that damage DNA and interrupt the cell cycle. While chemotherapy is a first-line treatment for many types of cancer, it also affects noncancerous cells, causing a range of adverse side-effects in patients, including hair follicle growth inhibition, gastrointestinal tract damage, peripheral neuropathy, and myelosuppression. These effects can be crippling and life-threatening, which limits the dosage of chemotherapy that is tolerable by cancer patients. As such, chemotherapeutic drugs must be administered within a narrow therapeutic window: at a dose sufficient to eliminate the cancer, but not so much as to excessively harm the patient (FIG. 1A). The therapeutic window is a criticial parameter of treatment, as ineffective therapy may leave behind a subset of malignant cells that will proliferate into recurrent (and often treatment-resistant) cancer.

To address this challenge, the modern strategy of targeted therapy uses molecules that interact with specific types of cells and cellular processes, with far fewer effects on other tissues. A major category is the use of monoclonal antibodies to selectively bind to proteins that are overexpressed on cancer cells, bringing along attached cytotoxic agents or triggering destruction by the immune system. This approach directs the effects of therapy towards cancer cells, decreasing toxicity. At the same time, however, the efficacy of targeted therapies are more constrained than their cytotoxic counterparts. Many malignant tumors do not exhibit targetable disease markers, and those that do often exhibit markers heterogeneously or at low levels. Targeted chemotherapy has the benefit of lower toxicity, albeit at the cost of limited efficacy, and so the therapeutic window is still relatively narrow.

The idea of using cytidine deaminases (CDA) to protect HSPCs from gemcitabine has been reported in literature before, with multiple papers overexpressing CDA in HSPCs. Lachmann et. al. 2013 gives a comprehensive review on all literature up to 2013 that has overexpressed CDA by either retroviral, plasmid, and lentiviral methods in cell lines (including fibroblast and hematopoetic cell lines), and in primary bone marrow cells and hematopoietic stem cells obtained from mice and humans. These papers show that CDA overexpression confers resistance against gemcitabine and other cytidine/deoxycytidine chemotherapy drugs such as ara-cytabine and decitabine.

Previous antibody or antibody fragment to enzyme conjugates exist such as antibody-directed enzyme prodrug therapy (ADEPT), targeted enzyme replacement fusions and neuroprotective protein fusions (Silver et. al. 2021). Out of these, ADEPT has been used for cancer treatment in which a bacterial enzyme that is known to convert a prodrug into its cytotoxic active form is delivered specifically to tumors via the conjugate where the activation happens extracellularly. The only successful ADEPT in clinical trials uses a bacterial enzyme, CPG2, and most efforts focus on optimizing this enzyme or finding alternatives that are less immunogenic (Sharma & Bagshawe, 2017). As such, there is a need in the art for new protein conjugates capable of altering the activity of chemotherapeutic agents.

### SUMMARY OF THE INVENTION

In general, the disclosure relates to the discovery of antigen-binding proteins conjugated to protection molecules to maintain both binding capacity and protection capacity. The antigen-binding proteins conjugated to protection molecules can be used to decrease the cytotoxic effects of a cytotoxic agent.

Certain aspects relate to compositions comprising an antigen-binding protein conjugated to a protection molecule. The protection molecule may be a molecule that is capable of neutralizing a cytotoxic agent. The protection molecule may neutralize the cytotoxic agent by any method, including enzymatic inactivation, degradation, sequestration, or otherwise acting as an antidote to the cytotoxic agent. The protection molecule may neturalize the cytotoxic agent by reducing the cytotoxic effects of the cytotoxic agent, including by providing an antidote. The protection molecule may induce cellular changes, such as signalling, gene expression, or protein-modifications, that reduce the cytotoxic effect of the cytotoxic agent. The protection molecule can be an cytotoxic agent-binding protein, including an antibody, or fragment thereof, that binds to the cytotoxic agent and/or an enzyme that binds to and neutralizes the cytotoxic agent. In certain aspects, the antigen-binding protein is an antibody or functional fragment thereof.

Disclosed herein are antigen-binding proteins capable of binding to an antigen on a healthy cell. The healthy cell may be a rapidly dividing cell. The healthy cell may be a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell. The healthy cell may be a cell that is affected by a cytotoxic agent, including any cytotoxic agent disclosed herein.

In certain aspects, the antigen-binding protein is capable of binding CD34. Antigen-binding proteins capable of binding CD34 are known in the art. In some aspects, the antigen-binding protein comprises one or more CDRs and/or a heavy chain and a light chain from an antibody known in the art to bind CD34. In certain aspects, the antigen-binding protein is capable of binding CD117. Antigen-binding proteins capable of binding CD117 are known in the art. In some aspects, the antigen-binding protein comprises one or more CDRs and/or a heavy chain and a light chain from an antibody known in the art to bind CD117, including those described in PCT Publication No. WO2020219775A1, which is hereby incorporated by reference in its entirety. In certain aspects, the antigen-binding protein is not capable of binding CD34. In certain aspects, the antigen-binding protein is not capable of binding CD117. In some aspects, the antigen-binding protein comprises one or more heavy chains disclosed in Table 1. In some aspects, the antigen-binding protein comprises one or more light chains disclosed in Table 1. In some aspects, the antigen-binding protein comprises a heavy chain disclosed in Table 1 and a light chain from the same antibody clone in Table 1. In some aspects, the antigen-binding protein comprises a heavy chain disclosed in Table 1 and a light chain from a different antibody clone in Table 1. Also disclosed are antigen-binding protein-cytidine deaminase conjugates comprising an amino acid sequence disclosed in Table 5.

Disclosed herein are antigen-binding proteins conjugated to protection molecules, where the protection molecules are enzymes. In certain aspects, the protection molecule may comprise a molecule capable of neutralizing gemcitabine. The protection molecule may be a cytidine deaminase. In some aspects, the cytidine deaminase comprises an amino acid sequence disclosed in Table 3. In certain aspects, the antigen-binding protein does not comprise one or more regions typically present on an antibody. The antigen-binding protein may or may not have an Fc region.

Also disclosed are proteins comprising an antigen-binding protein and a protection molecule. In some aspects, the protein comprises a sequence disclosed in Table 5. In some aspects, the protein comprises or does not comprise a purification tag. In some aspects, the purification tag is a 6×histidine tag. In some aspects, the 6×histidine tag comprises the sequence disclosed in Table 4.

Also disclosed herein are nucleic acids encoding for one or more proteins described herein. The nucleic acids can include one or more of the nucleic acids disclosed in Tables 1-4.

Disclosed herein are antigen-binding proteins conjugated to protection molecules that can neutralize cytotoxic agents, which may be anti-cancer agents. The cytotoxic agents can be any agent toxic to a cell in a patient, including an agent that kills a cell, such as an alkylating agent, an antimetabolite, a nucleotide analog, a taxane, a platinum-based agent, and/or an antibiotic. In certain aspects, the cytotoxic agent comprises gemcitabine. In certain aspects, the cytotoxic agent causes unwanted effects, including hair loss, fatigue, bruising, infection, anemia, gastrointestinal issues (such as nausea, vomiting, appetite changes, constipation, diarrhea), weight change, skin lesions, brain fog, change in libido, fertility issues, which may be reduced by the activity of the protection molecule. The cytotoxic agent may cause tissue degradation, including cardiac, pulmonary, liver, renal, gastrointestinal, or dermal tissue degradation.

The antigen-binding proteins described herein can be conjugated to the protection molecules described herein using any conjugation method. The protein and protection molecule can be conjugated using fusion chemistry, such as click chemistry, which may be copper-free click chemistry. The protein can be conjugated to the protection molecule at any suitable residue or moiety on the protein or protection molecule. The protein can be conjugated to the protection molecule at a primary amine on either the protein or protection molecule. In certain aspects, the antigen-binding protein and protection molecule are a single recombinant protein. The single recombinant protein may be generated by a nucleic acid that encodes both the antigen-binding protein and the protection molecule. The antigen-binding protein and protection molecule may be conjugated as a recombinant protein using an amino acid linker encoded in the nucleic acid. In certain aspects, the a heavy chain of the antigen-binding protein, a light chain of the antigen-binding protein, and the protection molecule are a single recombinant protein. The heavy chain, light chain, and antigen-binding protein may be separated by linkers, including a linker comprising one or more glycines and one or more serines such as a Glycine₄Serine linker. In some aspects, the linker comprises a linker disclosed in Table 2. In some aspects, the antigen-binding protein and the protection molecule are conjugated through one or more cognate affinity-binding molecules. The affinity-binding molecules can include molecules that can be appended to a protein and are able to bind to its cognate affinity-binding molecule. Examples of cognate affinity-binding molecule pairs include biotin and avidin or streptavidin, SpyTag and SpyCatcher, or other epitope tags and their cognate binding partner. The conjugation may occur anywhere on either the antigen-binding protein and/or protection molecule, including at the N-terminus and/or C-terminus of either molecule. The conjugation system may be a cleavable system. For example, the fusion chemistry may be reverable or contain cleavable moieties. The recombinant protein may comprise a cleavable sequence.

Certain aspects relate to compositions comprising any of the antigen-binding proteins described herein. The composition can also comprise other agents, including any cytotoxic agent described herein. The amount of cytotoxic agent in the composition can be an amount that is greater than an amount within the cytotoxic agent's therapeutic window. For example, the amount of cytotoxic agent in the composition can be an amount that is greater than what one skilled in the art would provide in the composition absent the antigen-binding protein. In certain aspects, the composition comprises an amount of cytotoxic agent of at least, at most, or approximately 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 mg (or any range derivable therein). In certain aspects, the composition comprises an amount of gemcitabine of at least, at most, or approximately 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 mg (or any range derivable therein). It is also contemplated herein that, in some aspects, one or more cytotoxic agents are not included in the composition.

Also described herein are methods including but not limited to methods of treating a patient, or methods of reducing one or more side effects of a cytotoxic agent, or methods of reducing cytotoxicity of a cytotoxic agent, or methods of expanding a therapeutic window of a cytotoxic agent, or methods of increasing the effective amount of a cytotoxic agent provided to a patient, any of which methods can comprise the step of administering to the patient any of the antigen-binding proteins conjugated to a protection molecule described herein. The patient can have received, or will receive the cytotoxic agent, before, during, or after being administered the antigen-binding protein. In certain aspects, the patient receives, or will receive, an amount of the cytotoxic agent that is greater than what one skilled in the art would administer to the patient absent the administration of the antigen-binding protein. In certain aspects, the patient has a disease, has been diagnosed with, has one or more symptoms of, or is suspected of having a disease, that is indicated for the cytotoxic agent. In certain aspects, the patient has, has been diagnosed with, has one or more symptoms of, or is suspected of having, a cancer, an infection, or an autoimmune disease. In certain aspects, the patient has had an adverse reaction to the cytotoxic agent. In certain aspects, the patient is prognosed to have an adverse reaction to the cytotoxic agent. In certain aspects, the patient is indicated to receive the cytotoxic agent.

In some aspects, the patient has or is likely to have one or more side effects from the cytotoxic agent. In some aspects, administering the antigen-binding protein conjugated to the protection molecule reduces the severity of or eliminates one or more side effects of the cytotoxic agent. In some aspects, the side effects comprise neutropenia, anemia, thrombocytopenia, lymphopenia, or a combination thereof. In some aspects, the side effects occur or are worsened when the patient receives the cytotoxic agent at a dosage above the therapeutic window of the cytotoxic agent. In some aspects, the side effects relate to the cytotoxic agent killing or affecting one or more healthy cells in the patient, including any of the healthy cells described herein.

Also disclosed are the following enumerated aspects:
Aspect 1 includes a composition comprising an antigen-binding protein conjugated to a protection molecule, wherein the protection molecule comprises a molecule capable of neutralizing a cytotoxic agent.
Aspect 2 depends upon Aspect 1, wherein the antigen-binding protein is an antibody or functional fragment thereof.
Aspect 3 depends upon Aspect 1 or 2, wherein the antigen-binding protein is capable of binding to an antigen expressed on a healthy cell.
Aspect 4 depends upon Aspect 3, wherein the healthy cell is a rapidly dividing healthy cell.
Aspect 5 depends upon Aspect 3 or 4, wherein the healthy cell is a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell.
Aspect 6 depends upon any one of Aspects 1 to 5, wherein the antigen-binding protein is capable of binding to CD34.
Aspect 7 depends upon any one of Aspects 1 to 5, wherein the antigen-binding protein is not capable of binding to CD34.
Aspect 8 depends upon any one of Aspects 1 to 5, wherein the antigen-binding protein is capable of binding to CD117.
Aspect 9 depends upon any one of Aspects 1 to 5, wherein the antigen-binding protein is not capable of binding to CD117.
Aspect 10 depends upon any one of Aspects 1 to 9, wherein the antigen-binding protein comprises a heavy chain amino acid sequence provided in Table 1 and a light chain amino acid sequence provided in Table 1, or a functional fragment thereof.
Aspect 11 depends upon any one of Aspects 1 to 10, wherein the antigen-binding protein and/or protection molecule comprises a purification tag.
Aspect 12 depends upon Aspect 11, wherein the purification tag comprises a 6×Histidine tag.
Aspect 13 depends upon Aspect 11 or 12, wherein the purification tag comprises a protease cleavage sequence.
Aspect 14 depends upon any one of Aspects 1 to 13, wherein the antigen-binding protein does not comprise an Fc region.
Aspect 15 depends upon any one of Aspects 1 to 14, wherein the protection molecule comprises an enzyme.
Aspect 16 depends upon any one of Aspects 1 to 15, wherein the protection molecule comprises a cytidine deaminase.
Aspect 17 depends upon Aspect 16, wherein the cytidine deaminase is a human cytidine deaminase.
Aspect 18 depends upon Aspect 16 or 17, wherein the cytidine deaminase comprises the amino acid sequence provided in Table 3.
Aspect 19 depends upon any one of Aspects 1 to 18, wherein the cytotoxic agent comprises an anti-cancer agent.
Aspect 20 depends upon any one of Aspects 1 to 19, wherein the cytotoxic agent comprises an alkylating agent, an antimetabolite, a nucleotide analog, a taxane, a platinum-based agent, and/or an antibiotic.
Aspect 21 depends upon any one of Aspects 1 to 20, wherein the cytotoxic agent comprises gemcitabine.
Aspect 22 depends upon any one of Aspects 1 to 21, wherein the antigen-binding protein and protection molecule are conjugated through click chemistry.
Aspect 23 depends upon Aspect 22, wherein the click chemistry is copper-free click chemistry.
Aspect 24 depends upon any one of Aspects 1 to 23, wherein the protection molecule is conjugated to a primary amine on the antigen-binding protein.
Aspect 25 depends upon any one of Aspects 1 to 21, wherein the antigen-binding protein and protection molecule are conjugated as a recombinant protein.
Aspect 26 depends upon any one of Aspects 1 to 21, wherein the antigen-binding protein and protection molecule are conjugated by cognate affinity-binding molecules.
Aspect 27 depends upon Aspect 26, wherein the cognate affinity-binding molecules comprise biotin and streptavidin and/or biotin and avidin.
Aspect 28 depends upon Aspect 26, wherein the cognate affinity-binding molecules comprise a Spy Tag and SpyCatcher.
Aspect 29 depends upon any one of Aspects 1 to 28, wherein the antigen-binding protein is cleavable from the protection molecule.
Aspect 30 depends upon any one of Aspects 1 to 29, wherein the protection molecule is conjugated to the N-terminus and/or C-terminus of the antigen-binding protein.
Aspect 31 includes a composition according to any one of Aspects 1 to 30, further comprising the cytotoxic agent.
Aspect 32 depends upon Aspect 31, wherein the composition comprises an amount of the cytotoxic agent greater than an amount within the cytotoxic agent's therapeutic window.
Aspect 33 includes a protein comprising a heavy chain amino acid sequence provided in Table 1, a light chain amino acid sequence provided in Table 1, and a cytidine deaminase sequence provided in Table 3.
Aspect 34 depends upon Aspect 33, further comprising a linker amino acid sequence provided in Table 2 and/or a His tag provided in Table 4.
Aspect 35 includes a CD117-binding protein-cytidine deaminase conjugate comprising an amino acid sequence provided in Table 5.
Aspect 36 includes a method of treating a patient that has, or will receive, a cytotoxic agent, the method comprising administering an antigen-binding protein conjugated to a protection molecule, wherein the protection molecule comprises a molecule capable of neutralizing the cytotoxic agent.
Aspect 37 depends upon Aspect 36, wherein the antigen-binding protein is an antibody or functional fragment thereof.
Aspect 38 depends upon Aspect 36 or 37, wherein the antigen-binding protein is capable of binding to an antigen expressed on a healthy cell.
Aspect 39 depends upon Aspect 38, wherein the healthy cell is a rapidly dividing healthy cell.
Aspect 40 depends upon Aspect 38 or 39, wherein the healthy cell is a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell.
Aspect 41 depends upon any one of Aspects 36 to 40, wherein the antigen-binding protein is capable of binding to CD34.
Aspect 42 depends upon any one of Aspects 36 to 40, wherein the antigen-binding protein is not capable of binding to CD34.
Aspect 43 depends upon any one of Aspects 36 to 40, wherein the antigen-binding protein is capable of binding to CD117.
Aspect 44 depends upon any one of Aspects 36 to 40, wherein the antigen-binding protein is not capable of binding to CD117.
Aspect 45 depends upon any one of Aspects 36 to 40, wherein the antigen-binding protein comprises a heavy chain amino acid sequence provided in Table 1 and a light chain amino acid sequence provided in Table 1, or a functional fragment thereof.
Aspect 46 depends upon any one of Aspects 36 to 45, wherein the antigen-binding protein and/or protection molecule comprises a purification tag.
Aspect 47 depends upon Aspect 46, wherein the purification tag comprises a 6×Histidine tag.
Aspect 48 depends upon Aspect 46 or 47, wherein the purification tag comprises a protease cleavage sequence.
Aspect 49 depends upon any one of Aspects 36 to 48, wherein the antigen-binding protein does not comprise an Fc region.
Aspect 50 depends upon any one of Aspects 36 to 49, wherein the protection molecule comprises an enzyme.
Aspect 51 depends upon any one of Aspects 36 to 50, wherein the protection molecule comprises a cytidine deaminase.
Aspect 52 depends upon Aspect 51, wherein the cytidine deaminase is a human cytidine deaminase.
Aspect 53 depends upon Aspect 51 or 52, wherein the cytidine deaminase comprises the amino acid sequence provided in Table 3.
Aspect 54 depends upon any one of Aspects 36 to 53, wherein the cytotoxic agent comprises an anti-cancer agent.
Aspect 55 depends upon any one of Aspects 36 to 54, wherein the cytotoxic agent comprises an alkylating agent, an antimetabolite, a nucleotide analog, a taxane, a platinum-based agent, and/or an antibiotic.
Aspect 56 depends upon any one of Aspects 36 to 55, wherein the cytotoxic agent comprises gemcitabine.
Aspect 57 depends upon any one of Aspects 36 to 56, wherein the antigen-binding protein and protection molecule are conjugated through click chemistry.
Aspect 58 depends upon Aspect 57, wherein the click chemistry is copper-free click chemistry.
Aspect 59 depends upon any one of Aspects 36 to 58, wherein the protection molecule is conjugated to a primary amine on the antigen-binding protein.
Aspect 60 depends upon any one of Aspects 36 to 56, wherein the antigen-binding protein and protection molecule are conjugated as a recombinant protein.
Aspect 61 depends upon any one of Aspects 36 to 56, wherein the antigen-binding protein and protection molecule are conjugated by cognate affinity-binding molecules.
Aspect 62 depends upon Aspect 61, wherein the cognate affinity-binding molecules comprise biotin and streptavidin and/or biotin and avidin.
Aspect 63 depends upon Aspect 61, wherein the cognate affinity-binding molecules comprise a Spy Tag and SpyCatcher.
Aspect 64 depends upon any one of Aspects 36 to 63, wherein the antigen-binding protein is cleavable from the protection molecule.
Aspect 65 depends upon any one of Aspects 36 to 64, wherein the protection molecule is conjugated to the N-terminus and/or C-terminus of the antigen-binding protein.
Aspect 66 depends upon any one of Aspects 36 to 65, wherein the patient received, or will receive, an amount of the cytotoxic agent that is greater than an amount in the cytotoxic agent's therapeutic window.
Aspect 67 depends upon any one of Aspects 36 to 66, wherein the patient is administered the antigen-binding protein prior to receiving the cytotoxic agent.
Aspect 68 depends upon any one of Aspects 36 to 67, wherein the patient is administered the antigen-binding protein concurrently with or in the same composition as the cytotoxic agent.
Aspect 69 depends upon any one of Aspects 36 to 68, wherein the patient is administered the antigen-binding protein subsequent to receiving the cytotoxic agent.
Aspect 70 depends upon any one of Aspects 36 to 69, wherein the patient has, has been diagnosed with, has one or more symptoms of, or is suspected of having a disease indicated for the cytotoxic agent.
Aspect 71 depends upon any one of Aspects 36 to 70, wherein the patient has, has been diagnosed with, has one or more symptoms of, or is suspected of having a cancer, an infection, or an autoimmune disease.
Aspect 72 depends upon any one of Aspects 36 to 71, wherein the patient has had an adverse reaction to the cytotoxic agent.
Aspect 73 depends upon any one of Aspects 36 to 72, wherein the patient is indicated to receive the cytotoxic agent.
Aspect 74 depends upon any one of Aspects 36 to 73, wherein the antigen-binding protein is administered by injection.
Aspect 75 depends upon any one of Aspects 36 to 74, wherein the antigen-binding protein is administered intravenously.
Aspect 76 includes a method of reducing one or more side effects of a cytotoxic agent, the method comprising administering to a patient an antigen-binding protein conjugated to a protection molecule, wherein the protection molecule comprises a molecule capable of neutralizing the cytotoxic agent.
Aspect 77 depends upon Aspect 76, wherein the antigen-binding protein is an antibody or functional fragment thereof.
Aspect 78 depends upon Aspect 76 or 77, wherein the antigen-binding protein is capable of binding to an antigen expressed on a healthy cell.
Aspect 79 depends upon Aspect 78, wherein the healthy cell is a rapidly dividing healthy cell.
Aspect 80 depends upon Aspect 78 or 79, wherein the healthy cell is a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell.
Aspect 81 depends upon any one of Aspects 76 to 80, wherein the antigen-binding protein is capable of binding to CD34.
Aspect 82 depends upon any one of Aspects 76 to 80, wherein the antigen-binding protein is not capable of binding to CD34.
Aspect 83 depends upon any one of Aspects 76 to 80, wherein the antigen-binding protein is capable of binding to CD117.
Aspect 84 depends upon any one of Aspects 76 to 80, wherein the antigen-binding protein is not capable of binding to CD117.
Aspect 85 depends upon any one of Aspects 76 to 80, wherein the antigen-binding protein comprises a heavy chain amino acid sequence provided in Table 1 and a light chain amino acid sequence provided in Table 1, or a functional fragment thereof.
Aspect 86 depends upon any one of Aspects 76 to 85, wherein the antigen-binding protein comprises a purification tag.
Aspect 87 depends upon Aspect 86, wherein the purification tag comprises a 6×Histidine tag.
Aspect 88 depends upon Aspect 86 or 87, wherein the purification tag comprises a protease cleavage sequence.
Aspect 89 depends upon any one of Aspects 76 to 88, wherein the antigen-binding protein does not comprise an Fc region.
Aspect 90 depends upon any one of Aspects 76 to 89, wherein the protection molecule comprises an enzyme.
Aspect 91 depends upon any one of Aspects 76 to 90, wherein the protection molecule comprises a cytidine deaminase.
Aspect 92 depends upon Aspect 91, wherein the cytidine deaminase is a human cytidine deaminase.
Aspect 93 depends upon Aspect 91 or 92, wherein the cytidine deaminase comprises the amino acid sequence provided in Table 3.
Aspect 94 depends upon any one of Aspects 76 to 93, wherein the cytotoxic agent comprises an anti-cancer agent.
Aspect 95 depends upon any one of Aspects 76 to 94, wherein the cytotoxic agent comprises an alkylating agent, an antimetabolite, a nucleotide analog, a taxane, a platinum-based agent, and/or an antibiotic.
Aspect 96 depends upon any one of Aspects 76 to 95, wherein the cytotoxic agent comprises gemcitabine.
Aspect 97 depends upon any one of Aspects 76 to 96, wherein the antigen-binding protein and protection molecule are conjugated through click chemistry.
Aspect 98 depends upon Aspect 97, wherein the click chemistry is copper-free click chemistry.
Aspect 99 depends upon any one of Aspects 76 to 98, wherein the protection molecule is conjugated to a primary amine on the antigen-binding protein.
Aspect 100 depends upon any one of Aspects 76 to 96, wherein the antigen-binding protein and protection molecule are conjugated as a recombinant protein.
Aspect 101 depends upon any one of Aspects 76 to 96, wherein the antigen-binding protein and protection molecule are conjugated by cognate affinity-binding molecules.
Aspect 102 depends upon Aspect 101, wherein the cognate affinity-binding molecules comprise biotin and streptavidin and/or biotin and avidin.
Aspect 103 depends upon Aspect 101, wherein the cognate affinity-binding molecules comprise a SpyTag and SpyCatcher.
Aspect 104 depends upon any one of Aspects 76 to 103, wherein the antigen-binding protein is cleavable from the protection molecule.
Aspect 105 depends upon any one of Aspects 76 to 104, wherein the protection molecule is conjugated to the N-terminus and/or C-terminus of the antigen-binding protein.
Aspect 106 depends upon any one of Aspects 76 to 105, wherein the patient received, or will receive, an amount of the cytotoxic agent that is greater than an amount in the cytotoxic agent's therapeutic window.
Aspect 107 depends upon any one of Aspects 76 to 106, wherein the patient is administered the antigen-binding protein prior to receiving the cytotoxic agent.
Aspect 108 depends upon any one of Aspects 76 to 107, wherein the patient is administered the antigen-binding protein concurrently with or in the same composition as the cytotoxic agent.
Aspect 109 depends upon any one of Aspects 76 to 108, wherein the patient is administered the antigen-binding protein subsequent to receiving the cytotoxic agent.
Aspect 110 depends upon any one of Aspects 76 to 109, wherein the patient has, has been diagnosed with, has one or more symptoms of, or is suspected of having a disease indicated for the cytotoxic agent.
Aspect 111 depends upon any one of Aspects 76 to 110, wherein the patient has, has been diagnosed with, has one or more symptoms of, or is suspected of having a cancer, an infection, or an autoimmune disease.
Aspect 112 depends upon any one of Aspects 76 to 111, wherein the patient has had an adverse reaction to the cytotoxic agent.
Aspect 113 depends upon any one of Aspects 76 to 112, wherein the patient is indicated to receive the cytotoxic agent.
Aspect 114 depends upon any one of Aspects 76 to 113, wherein the antigen-binding protein is administered by injection.
Aspect 115 depends upon any one of Aspects 76 to 114, wherein the antigen-binding protein is administered intravenously.
Aspect 116 depends upon any one of Aspects 76 to 115, wherein the side effects comprise neutropenia, anemia, thrombocytopenia, lymphopenia, or a combination thereof.
Aspect 117 depends upon any one of Aspects 76 to 116, wherein the side effects occur or are worsened when the patient receives the cytotoxic agent at a dosage above the therapeutic window of the cytotoxic agent.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Molecularly-targeted chemoprotection widens the therapeutic window of cytotoxic chemotherapy by selectively protecting healthy tissues from toxicity. FIG. 1A shows the therapeutic window between efficacy of a drug and toxicity of a drug. FIG. 1B shows a schematic for the antigen-binding proteins conjugated to protection molecule concept.

FIGS. 2A-2C. FIG. 2A shows a specific example of an antigen-binding protein conjugated to a protection molecule. CD34 is a surface protein commonly expressed on stem and progenitor cells, with low expression on solid tumors. An antibody targeting CD34 is conjugated to cytidine deaminase, an enzyme that degrades the chemotherapeutic drug (CTX) gemcitabine (FIG. 2B). FIG. 2C shows an example of copper-free click chemistry and NHS ester chemistry used to build a conjugate exemplified herein.

FIGS. 3A-3B. Cytidine deaminase (CDA) is able to protect mammalian cells from gemcitabine toxicity (FIG. 3A) to levels indistinguishable from negative control. Anti-CD34 and anti-EpCAM Antibodies against CD34⁺ and EpCAM⁺ cell lines are used to demonstrate molecularly-targeted chemoprotection in vitro (FIG. 3B).

FIG. 4. Enzyme activity assay results for antibodies conjugated to CDA, normalized to the original activity of unconjugated CDA and background control.

FIG. 5. A representation of a fusion protein described herein. VH: heavy chain variable fragment of CD117 antibody. VL: light chain variable fragment of CD117 antibody. (G4S)3: glycine serine linker. CDA: human cytidine deaminase.

FIG. 6. A representation of a fusion protein described herein showing subunits. Heavy chain variable fragment of CD117 antibody, glycine, serine linker, light chain variable fragment of CD117 antibody, cytidine deaminase, and 6× histidine tag are shown.

FIG. 7. Flow cytometry results for CD117 in ovarian cancer, breast cancer, and pancreatic cancer cell lines.

FIG. 8. Extracellular CDA can reduce gemcitabine-mediated cell killing; protection against gemcitabine is extracellular CDA dose-dependent. This indicates when our fusion protein with CDA is in the extracellular space, it should be able to inactivate gemcitabine without the need to be internalized.

FIGS. 9A-9E. Exemplary production of cytidine deaminase fusion protein. FIG. 9A shows that the scFv_CD117_CDA_His fusion protein is produced in the induced lysate. FIG. 9B shows a Western blot of nickel resin-purified T7 shuffle lysate. S1: flow through after resin binding. W1: initial wash with equilibration buffer. WW1: second wash with equilibration buffer and lower concentration elution buffer. E #: eluted fractions. E2 is 1.75 ug/mL CDA. FIG. 9C shows CDA concentrations in T7 shuffle lysate purified samples. FIG. 9D shows absorbance results from indirect ELISA of a fusion protein binding to CD117. FIG. 9E shows results from CDA activity assay of a fusion protein.

FIG. 10. An image of a Western blot for 6×Histidine of proteins produced by T7 Shuffle cells under three different induction conditions: 1) 30° C. for 4 hours; 2) 25° C. for 24 hours; and 3) 30° C. for 4 hours and 25° C. for 20 hours. The single chain variable fragment (scFv) of human CD117 antibody with the cytidine deaminase (CDA) enzyme was produced in T7 Shuffle cells, the recombinant fusion protein expected at about 48 kDa.

FIG. 11. Confirming the binding of the fusion protein to the target CD117. Absorbance results are shown from an indirect ELISA against CD117. Lysates from T7 Shuffle cells that were induced under different conditions were assayed to confirm the binding of the fusion protein to the target. Uninduced lysate was used as negative control and about 4× higher signal from induced lysate can be observed.

FIG. 12. An exemplary graphical representation of absorbance spectra shift from 271 nm to 262 nm, the indicative peaks of cytidine to uridine, to confirm the activity of CDA in a fusion protein.

FIGS. 13A-13F show CDA results of lysates of T7 Shuffle cells that were induced for 4 hours for scFv-CDA-His fusion protein. FIG. 13A shows an image of a Western blot for anti-6×Histine in lysate. FIG. 13B shows Western blot images of anti-6×Histine and anti-CDA in T7 Shuffle cell lysate samples from various stages of nickel resin purification. FIG. 13C shows a Western blot for anti-CDA in samples for 0, 25, 50, 75, 100, 500, and 1000 μg of CDA. FIG. 13D is a CDA standard curve on Western blot. FIG. 13E is a graphical representation of area over CDA in pg for the standards shown in FIG. 13D and tested samples. FIG. 13F is a graphical representation of CDA concentration in purified T7 Shuffle cell lysate samples. S1: flow through after resin binding. W1: initial wash with equilibration buffer. WW1: second wash with equilibration buffer and lower concentration elution buffer. E #: eluted fractions. E2 is 1.75 ug/mL CDA.

FIGS. 14A-14E show CDA activity and CD117 binding results of eluted fractions from T7 Shuffle cell lysates that were induced for 4 hours with a scFV-CDA-His fusion protein. FIG. 14A is a schematic diagram of an indirect ELISA procedure. FIG. 14B illustrates the chemical reactions in CDA activity assay. FIG. 14C is a cytidine standard curve graph. FIG. 14D is a graphical representation of binding affinity of a fusion protein to CD117. FIG. 14E is a graphical representation of CDA activity of a fusion protein.

FIGS. 15A-15D show images of SDS-PAGE gels for confirming the production of proteins of interest in BL21 DE3 and T7 Shuffle cells. FIG. 15A is an image of a SDS-PAGE gel of lysates of BL21 DE3 that were induced under 37° C. or 30° C., for 4 hours, 7 hours, or overnight, for scFv-His. FIG. 15B is an image of a SDS-PAGE gel of lysates of BL21 DE3 that were induced under 37° C. or 30° C., for 4 hours, 7 hours, or overnight, for CDA-His. * marks an erroneous sample where the correct 30 C 7 h sample is shown on the far right marked *. FIG. 15C is an image of a SDS-PAGE gel of lysates of BL21 DE3 that were induced under 37° C. or 30° C., for 4 hours, 7 hours, or overnight, for scFv-CDA-His. ** marks an erroneous sample where the correct 30 C 7 h is shown on the far right marked **. FIG. 15D is an image of a SDS-PAGE gel of lysates of T7 Shuffle that were induced under 30° C. for 4 hours or overnight, for scFv-His, CDA-His, or scFv-CDA-His.

FIGS. 16A-16C show images of SDS-PAGE gels for confirming the yield of protein after purification, and for the purity of each sample. FIG. 16A is an image of a SDS-PAGE gel of purified protein samples from lysates of BL21 DE3 that were induced at 37° C. or 30° C., for 4 hours, 7 hours, or overnight, for scFv-His or scFv-CDA-His. FIG. 16B is an image of a SDS-PAGE gel of purified protein samples from lysates of BL21 DE3 that were induced at 37° C. or 30° C., for 4 hours, 7 hours, or overnight, for CDA-His or scFv-CDA-His. FIG. 16C is an image of a SDS-PAGE gel of purified protein samples from lysates of T7 Shuffle that were induced at 30° C. for 4 hours or overnight, for scFv-His, CDA-His, or scFv-CDA-His.

FIGS. 17A-17D show the determination of purity of samples by densitometry analysis on SDS-PAGE gels. FIG. 17A is an image of a SDS-PAGE gel of lysates from BL21 DE3 that were induced for 4 hours, 7 hours, or overnight, at 37° C. or 30° C., for scFv-His or scFv-CDA-His. FIG. 17B is a graphical representation of intensity of the ladder bands in lane 1 shown in FIG. 17A. FIG. 17C is a graphical representation of intensity of the bands in lane 7 shown in FIG. 17A. FIG. 17D shows results from a densitometry analysis of lane 7, emphasizing the bange percentages and band number 11.

FIGS. 18A-18B are graphical representations of the purity and concentration of protein samples determined by densitometry analysis on SDS-PAGE gels. FIG. 18A shows the percent purity of scFv-His, CDA-His, and scFv-CDA-His induced in BL21 DE3 or T7 Shuffle over 4, 7, or overnight, at 37° C. or 30° C. Purity of samples ranged from about 5-85%. FIG. 18B shows the ceoncentration in μg/mL of scFv-His, CDA-His, and scFv-CDA-His induced in BL21 DE3 or T7 Shuffle over 4, 7, or overnight, at 37° C. or 30° C. The amount of protein can be calculated by percent purity multiplied by total protein concentration in the sample measured by Qubit. High range of protein concentration is shown, with the yield between about 4-450 μg/mL.

FIGS. 19A-19F show representative Western blot images validating the presence of scFv-His, CDA-HIS, or scFv-CDA-His in BL21 and T7 Shuffle cells by using an anti-6×Histidine antibody. FIG. 19A shows a Western blot image of scFv-His in BL21 cells that were induced for 4 hours, 7 hours, or overnight, at 37° C. or 30° C. FIG. 19B shows a Western blot image of scFv-His in T7 Shuffle that were induced overnight or for 4 hours at 30° C. FIG. 19C shows a Western blot image of CDA-His in BL21 cells that were induced for 4 hours, 7 hours, or overnight, at 37° C. or 30° C. FIG. 19D shows a Western blot image of CDA-His in T7 Shuffle that were induced overnight or for 4 hours at 30° C. FIG. 19E shows a Western blot image of scFv-CDA-His in BL21 cells that were induced for 4 hours, 7 hours, or overnight, at 37° C. or 30° C. FIG. 19F shows a Western blot image of CDA-His in T7 Shuffle that were induced overnight or for 4 hours at 30° C.

FIGS. 20A-20B show the activity of recombinantly produced CDA-His measured by using the indophenol method. FIG. 20A is a graphical representation of ammonia produced in μM. FIG. 20B is a graphical representation of the mean specific CDA activity of CDA-His that were induced in BL21 DE3 for 4 hours, 7 hours, or overnight at 37° C. or 30° C. A commercially available CDA is provided as a positive control.

FIG. 21 is a graphical representation of the binding of recombinantly produced scFv-His and scFv-CDA-His to CD117 by using indirect CD117 ELISA. Protein samples are from BL21 DE3 and T7 Shuffle that were induced for 4 hours, 7 hours, or overnight at 37° C. or 30° C.

### DESCRIPTION OF ILLUSTRATIVE ASPECTS

Though previous literature has shown overexpression of CDA in HSPCs leads to less killing by gemcitabine, the conjugated proteins described herein provide novel therapeutics, which may be injected, that can deliver the enzyme directly. With the previous overexpression methods, the HSPCs would first need to collected from the patient, then genetically modified ex vivo, and transplanted back into the patient. The compositions and methods and other aspects described herein beneficially do not require any cell collection or ex vivo modification, making such aspects more easily translatable to a clinical setting.

Comparing the aspects herein to the ADEPT technology and other previous methods, said aspects are fundamentally different in at least two ways. First, the aspects target healthy cells and not cancer cells. Previous methods required identifying tumor-specific antigens, which can be especially challenging due to 1) the low abundance of such antigens in solid tumors versus liquid tumors, 2) the tumors' ability to alter the expression of their surface antigens to evade immune system attack, 3) the heterogeneous expression of the antigen within and between individuals, 4) the physical microenvironment barriers such as the dense stroma restricting infiltration and migration of molecules, and 5) the immunosuppressive tumor microenvironment inhibiting the activity of delivered molecules. The aspects described herein can overcome this antigen identification problem as 1) all individuals' HPSCs have the same antigens, eliminating the need for personalized treatment, 2) healthy cells do not alter the expression of their antigens as frequently as cancer cells, and 3) the bone marrow does not impose a physical barrier to complexes as it does not have a dense stroma/extracellular matrix. Second, aspects herein do not require delivering bacterial enzymes but may include delivery of human enzymes. Clinical trials for ADEPT showed high immunogenicity in patients receiving the treatment and the patients had developed antibodies against the bacterial enzymes. In aspects herein that deliver human-origin enzymes, low immunogenicity is expected.

### I. DEFINITIONS

The terms “protein,” “polypeptide,” and “peptide” are used interchangeably herein when referring to a gene product.

The term “antigen-binding protein” refers to a protein that specifically binds to a certain antigen. In some aspects, the antigen-binding protein has no, undetectable, or minimal non-specific binding. The certain antigen may be CD34.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

Cells or a culture of cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

Cells or a culture of cells are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In some aspects, the methods are useful for reducing the size and/or cell number of a tumor. In some aspects, the method of the disclosure are useful for inhibiting the growth of tumors, such as solid tumors, in a subject.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The antibody may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The term functional antibody fragment includes antigen binding fragments of antibodies, including e.g., Fab′, F(ab′)2, Fab, Fv, rlgG, and scFv fragments. The term scFv refers to a single chain Fv antibody in which the variable domains of the heavy chain and of the light chain of a traditional two chain antibody have been joined to form one chain. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment retains the ability to bind its cognate antigen at comparable affinity to the full antibody.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain aspects, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include several different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition may be adjusted according to well-known parameters.

The term “unit dose” or “dosage” refers to physically discrete units suitable for use in a subject, each unit containing a predetermined quantity of the therapeutic composition calculated to produce the desired responses discussed herein in association with its administration, i.e., the appropriate route and treatment regimen. The quantity to be administered, both according to number of treatments and unit dose, depends on the effect desired. The actual dosage amount of a composition of the present aspects administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any particular dose derivable therein. In non-limiting examples of a range derivable from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The use of a single chain variable fragment (scFv) is of particular interest. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference. Described herein are BCMA-specific scFv molecules that comprise the variable regions of light and heavy immunoglobulin chains encoding BCMA-binding domains that are engineered into a single polypeptide. Similarly, the CS1-specific scFv molecules described herein comprise the variable regions of light and heavy immunoglobulin chains encoding CS1-binding domains that are engineered into a single polypeptide.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

Subject” and “patient” refer to either a human or non-human, such as primates, mammals, and vertebrates. In particular aspects, the subject is a human.

Throughout this application, the term “about” is used to indicate that a value includes the inherent variation of error for the device, the method being employed to determine the value, or the variation that exists among the study subjects.

### II. ANTIGEN-BINDING PROTEIN CONJUGATES

Aspects of the disclosure relate to antigen-binding proteins conjugated to a protection molecule. In some aspects, the antigen-binding protein comprises an anti-CD34 antibody or a fragment thereof. In some aspects, the antigen-binding protein comprises an anti-CD117 antibody or a fragment thereof. In some aspects, the antigen-binding protein does not comprise an anti-CD34 antibody or a fragment thereof. In some aspects, the antigen-binding protein does not comprise an anti-CD117 antibody or a fragment thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies. The antigen may be expressed on a healthy cell. The healthy cells, such as rapidly dividing cells, may be susceptible to cytotoxic agents. The healthy cells may be cells that have not transformed to neoplastic cells. In some aspects, the antigen is a CD34 protein.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4:302; 2013)

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgAQ1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some aspects, bispecific antibodies can be biparatopic, wherein a bispecific antibody may specifically recognize a different epitope from the same antigen. In some aspects, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some aspects, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for aspects of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some aspects, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### 1. Functional Antibody Fragments and Antigen-Binding Fragments

a. Antigen-Binding Fragments

Certain aspects relate to antibody fragments, such as antibody fragments comprise the antigen-binding proteins described herein. The term functional antibody fragment includes antigen-binding fragments of an antibody that retain the ability to specifically bind to an antigen. These fragments are constituted of various arrangements of the variable region heavy chain (VH) and/or light chain (VL); and in some aspects, include constant region heavy chain 1 (CH1) and light chain (CL). In some aspects, they lack the Fc region constituted of heavy chain 2 (CH2) and 3 (CH3) domains. Aspects of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv) 2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv) 2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

b. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included. The Fc region may be removed from an antibody to generate the antigen-binding proteins described herein. Removing the Fc region may allow for the antigen-binding protein to not induce an immune response against the antigen, or cell expressing the antigen, the antigen-binding protein targets.

c. Polypeptides with Antibody CDRs & Scaffolding Domains that Display the CDRs

Antigen-binding peptide scaffolds, such as complementarity-determining regions (CDRs), are used to generate protein-binding molecules in accordance with the aspects. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### III. CELLS

Certain aspects relate to cells comprising polypeptides or nucleic acids of the disclosure, such as any of the antigen-binding proteins described herein. In some aspects the cell is an immune cell or a T cell. “T cell” includes all types of immune cells expressing CD3 including T-helper cells, cytotoxic T-cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual. In some aspects, the cell used in therapy of a patient is autologous. In some aspects, the cell used in therapy of a patient is non-autologous.

### IV. METHODS FOR MODIFYING GENOMIC DNA

In certain embodiments, the genomic DNA is modified either to include additional mutations, insertions, or deletions, or to integrate certain molecular contructs of the disclosure so that the constructs are expressed from the genomic DNA. In some aspects, a nucleic acid encoding a polypeptide of the disclosure is integrated into the genomic DNA of a cell. In some aspects, the integration is targeted integration. In some aspects, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T-cell receptor alpha constant) locus. For instance, cells would first be electroporated with a ribonucleoprotein (RNP) complex consisting of Cas9 protein complexed with a single-guide RNA (sgRNA) targeting the TRAC (T-cell receptor alpha constant) locus. Fifteen minutes post electroporation, the cells would be treated with AAV6 carrying the HDR template that encodes for the CAR.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxbl-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRIPSR/Cas-like endonucleases, I-Tev1 nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In aspects comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1): 93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some aspects, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one aspect, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### I. Methods of Treatment

Aspects of the current disclosure relate to methods of treating a patient that has, or will receive, a cytotoxic agent. Also contemplated are aspects that relate to methods of reducing cytotoxicity of a cytotoxic agent. The immune response stimulation may be done in vitro, in vivo, or ex vivo. The methods generally involve administering an antigen-binding protein conjugated to a protection molecule. In some aspects, the antigen-binding protein is a genetically modified mammalian cell with an expression vector, or an RNA (e.g., in vitro transcribed RNA), comprising nucleotide sequences encoding the antigen-binding protein. The cell can be an immune cell (e.g., a T lymphocyte or NK cell), a stem cell, a progenitor cell, etc. In some aspects, the cell is a cell described herein or the progeny thereof.

In some aspects, the methods relate to administration of the antigen-binding protein in combination with a treatment for cancer (or other disease treated with a cytotoxic agent) or administration to a person with a cancer (or other disease treated with a cytotoxic agent).

### II. Pharmaceutical Compositions

Administration of the compositions according to the current disclosure will typically be via any common route. This includes, but is not limited to parenteral, orthotopic, intradermal, subcutaneous, intramuscular, intraperitoneal, or intravenous injection.

Typically, compositions of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 2, 3, 4, 5, 6, 7, 8, 9, 10 or more. The administrations may range from 2-day to 12-month intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### V. CYTOTOXIC AND/OR ADDITIONAL THERAPIES

The current methods and compositions of the disclosure may include one or more additional therapies known in the art and/or described herein, which may be the cytotoxic agent protected by the protection molecule. In some aspects, the cytotoxic and/or additional therapies or agents comprise an additional cancer treatment. Examples of such treatments are described herein.

### A. Immunotherapies

In some aspects, the additional therapy or agent comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

In some aspects, the additional therapy or agent comprises immune checkpoint inhibitors. Certain aspects are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### B. Oncolytic Virus

In some aspects, the additional therapy or agent comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### C. Polysaccharides

In some aspects, the additional therapy or agent comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### D. Neoantigens

In some aspects, the additional therapy or agent comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### E. Chemotherapies

In some aspects, the additional therapy or agent or agent comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

In some aspects, the additional therapy or agent or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

### VI. ADMINISTRATION OF THERAPEUTIC COMPOSITIONS

Methods of the disclosure include administration of a combination of therapeutic agents and/or administration of therapeutic agents, such as the antigen-binding protein described herein and cytotoxic agent, for example. The therapy may be administered in any suitable manner known in the art. For example, the therapies may be administered sequentially (at different times) or concurrently (at the same time). In some aspects, the therapies are in a separate composition. In some aspects, the therapies are in the same composition.

Various combinations of the therapies may be employed, for example, one therapy designated “A” and another thapy designated “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies of the disclosure may be administered by the same route of administration or by different routes of administration. In some aspects, the therapy is administered intracolonically, intravenously, intramuscularly, subcutaneously, topically, orally, transdermally, intraperitoneally, intraorbitally, by implantation, by inhalation, intrathecally, intraventricularly, or intranasally. In some aspects, the microbial modulator is administered intravenously, intramuscularly, subcutaneously, topically, orally, transdermally, intraperitoneally, intraorbitally, by implantation, by inhalation, intrathecally, intraventricularly, or intranasally.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some aspects, the therapeutically effective or sufficient amount of a therapeutic composition that is administered to a human will be in the range of about 0.01 to about 50 mg/kg of patient body weight whether by one or more administrations. In some aspects, the therapeutic agent used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some aspects, the therapeutic agent is administered at 15 mg/kg. However, other dosage regimens may be useful. In one aspect, a therapeutic agent described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50μ; or about 1μ M to 40μ; or about 1μ M to 30μ; or about 1μ M to 20μ; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VII. KITS

Certain aspects of the disclosure also encompass kits for performing the methods of the disclosure. Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit aspects relate to kits comprising the therapeutic compositions of the disclosure. The kits may be useful in the treatment methods of the disclosure and comprise instructions for use.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

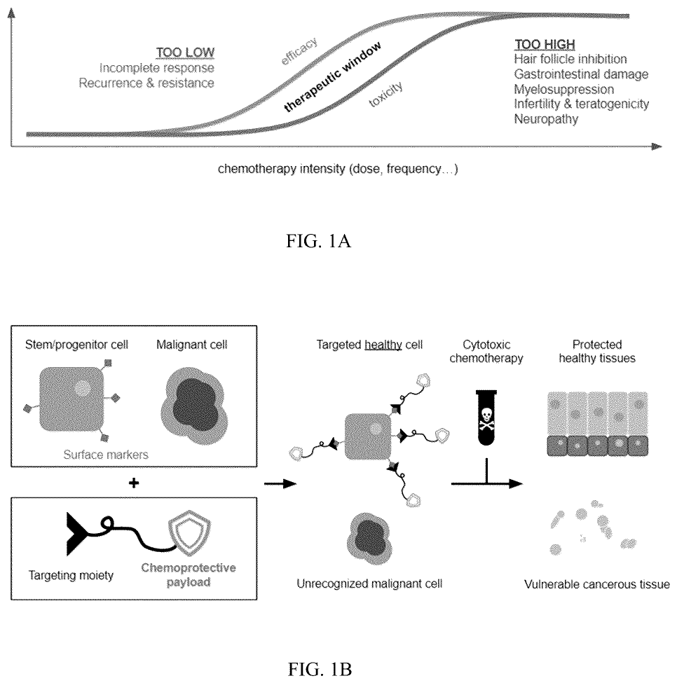

Described herein are strategies for cancer treatment that widens the therapeutic window of chemotherapy by directly and selectively protecting healthy tissues from cytotoxic drugs. The approach, in certain embodiments called molecularly-targeted chemoprotection (MTC), inverts the targeted therapy paradigm to instead deliver chemoprotective compounds to cells expressing molecular markers of healthy tissues prior to chemotherapy (FIG. 1B). Stem and progenitor cells are particularly critical for tissue maintenance and highly susceptible to cytotoxic therapy. With prudent selection of molecular markers to target, these cells can be protected from cytotoxic drugs more so than malignant cells. Targeting mechanisms can be configured, such as the use of antibodies (monoclonal or polyclonal), aptamers, or affimers. The mechanism of chemoprotection can also be tailored to different diseases and treatments, such as the use of drug-degrading enzymes, drug sequestrants, or nanoparticles of “antidote”. This flexible strategy widens the therapeutic window of cytotoxic drugs, making chemotherapy more tolerable for patients and enabling the use of higher-intensity treatment regimens to more completely and expeditiously treat cancer.

An example of MTC (FIGS. 1A-1B) uses an antibody fragment targeted against CD34-expressing cells that is covalently conjugated to cytidine deaminase (CDA), an enzyme that degrades gemcitabine (a commonly prescribed cytotoxic chemotherapy). CD34 is expressed on many stem and progenitor cells with low expression on solid tumors, altogether making it a strong candidate for targeted protection. The use of a CD34-recognizing antibody fragment without an Fc region ensures that the conjugate will not induce an immune response against the targeted healthy tissue. The conjugate can be administered prior to gemcitabine therapy, enriching levels of cytidine deaminase in CD34+ tissues. This approach widens the therapeutic window to make higher gemcitabine doses more tolerable for patients.

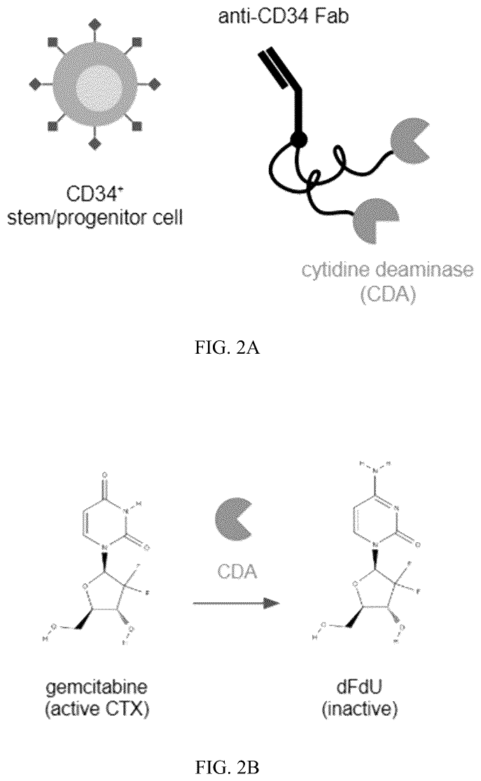

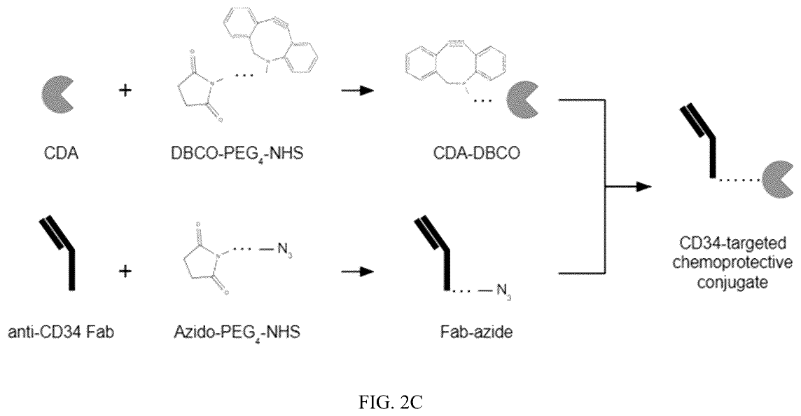

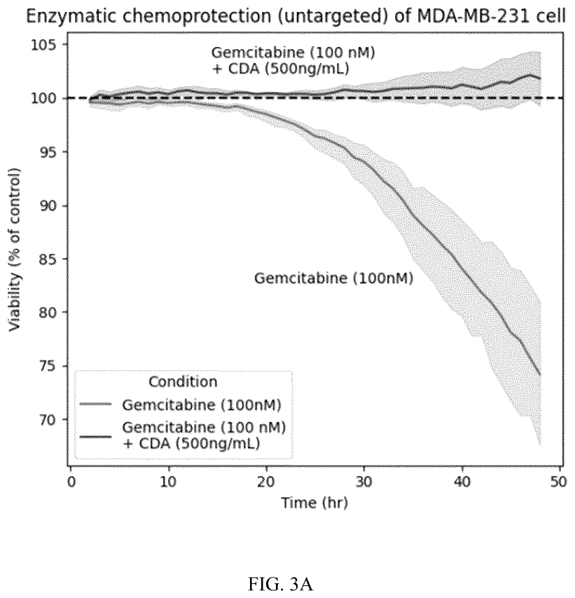

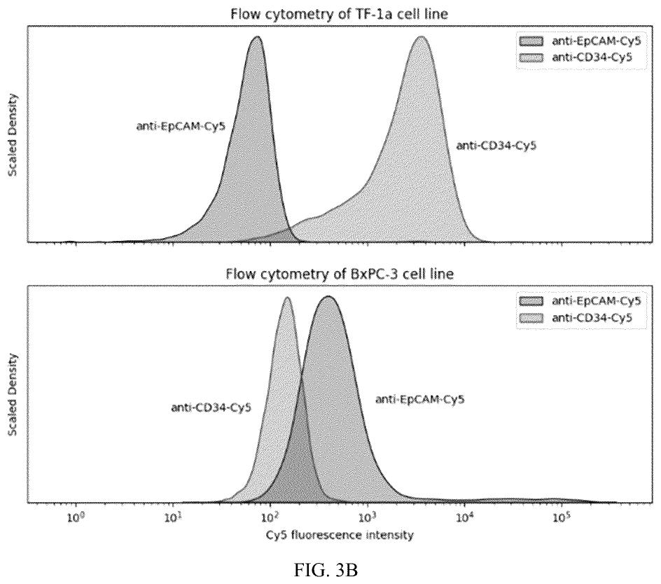

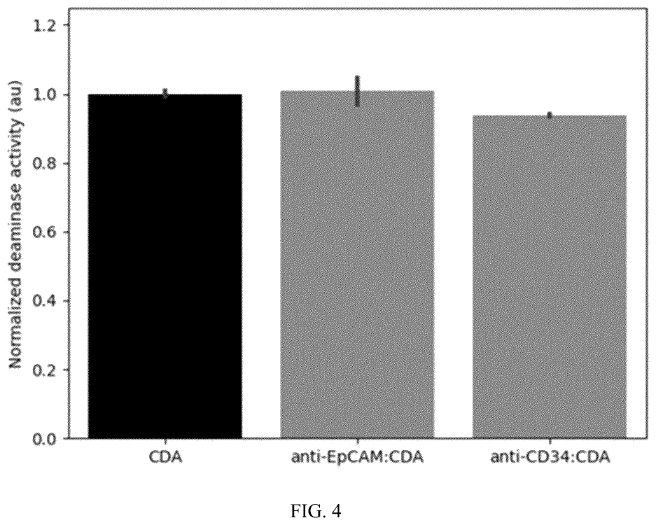

CDA and anti-CD34 antibodies can be produced recombinantly, either as separate products or as a fusion protein. In one example, copper-free click chemistry was with NHS ester functionalization of primary amines on the two molecules to produce a CD34-targeted chemoprotective conjugate for use in in vitro experiments (FIG. 2C). To validate, the unconjugated CDA in solution was able to protect a mammalian cell line from gemcitabine toxicity to levels indistinguishable from the negative control (FIG. 3A). For validation of targeted protection, two cell lines were used, one expressing CD34 (TF-la, derived from leukemia cells) and one expressing EpCAM (BxPC-3, derived from pancreatic cancer). The anti-CD34 and anti-EpCAM (control) antibodies that were obtained can indeed distinguish between these two cell lines (FIG. 3B). The generated conjugates were analyzed in a cytidine deaminase activity assay, and compared to unconjugated CDA. The antibody conjugates were purified with size-exclusion at a nominal 100 kDa MWCO, which is considerably larger than CDA (even in its multimeric forms). So all unconjugated CDA was removed for these samples. As shown in FIG. 4, the antibody conjugates had activity equivalent to unconjugated CDA.

### Example 2

CD117 is a receptor commonly found on the surface of hematopoietic stem and progenitor cells and has been used in literature to specifically ablate bone marrow cells prior to bone marrow transplantation such as in Czechowicz et al. 2019 and Pang et al. 2019. CDA is an important enzyme in the pyrimidine salvaging pathway and is known to deaminase cytidine/deoxycytidine (and their analogs) into uridine/deoxyuridine. Therefore, it can deaminate and therefore inactivate gemcitabine, a deoxyctidine analog used as a common chemotherapeutic. By fusing the scFv with the CDA, the enzyme can be delivered specifically to HSPCs prior to gemcitabine administration, leading to protection of the HSPCs from chemotherapy-induced killing. With the protection of HSPCs, the inventors expect to prevent myelosuppression, one of the most common side effects of chemotherapy. Depletion of the HSPCs also leads to reduction in downstream cell lineages in chemotherapy patients and therefore manifests as neutropenia (decreased white blood cells), anemia (decreased red blood cells), thrombocytopenia (decreased platelets) and lymphopenia (decreased lymphocytes). By protecting the HSPCs, reduction in these cell populations may be prevented as well as alleviation of a wide range of side effects caused by chemotherapy.

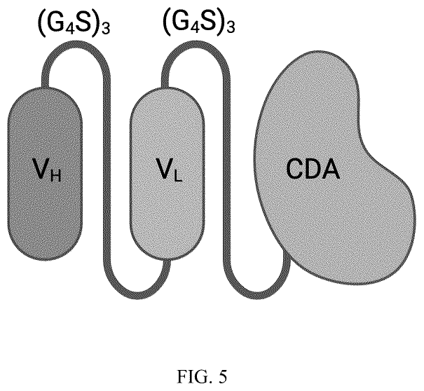

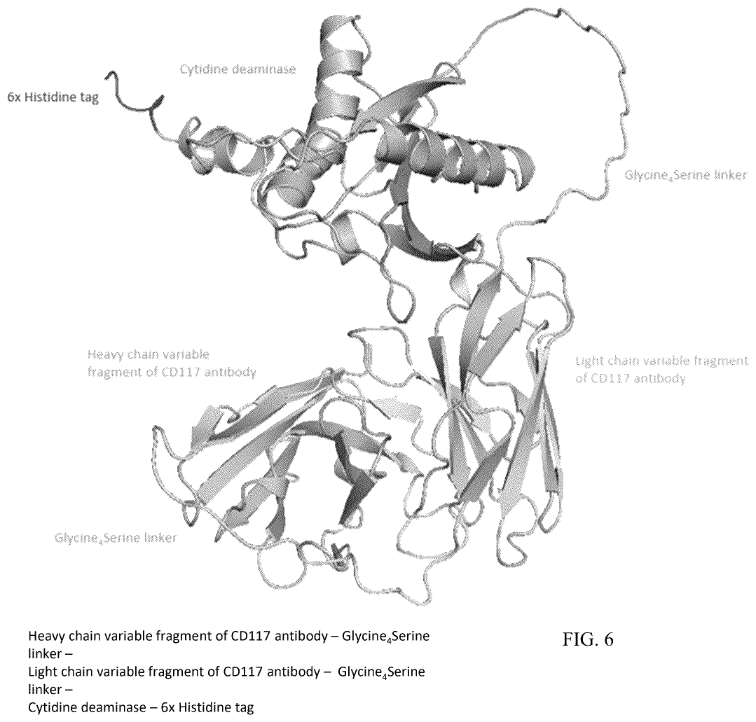

A fusion protein comprised of a single chain variable fragment (scFv) of the human CD117 (i.e. stem cell factor receptor) antibody and the enzyme cytidine deaminase (CDA) is described herein. The scFv sequence includes the heavy and light variable regions of CD117 antibody which are connected to each other through a glycine-serine linker (FIGS. 5-6). The scFv is then followed by a second linker that separates it from the human cytidine deaminase sequence (FIGS. 5-6). The inventors used a glycine-serine linker again between the scFv and the CDA, therefore producing the fusion protein with one plasmid in one expression system. However, the fusion protein can also be developed by producing the scFv and CDA separately with either chemical modifications on each to facilitate conjugation (which includes but is not limited to adding a click chemistry handle on one protein and an alkyne group on the other) or by adding additional cognate sequences to our proteins such as biotin and avidin or SpyTag and SpyCatcher.

In some aspects, for purification purposes, the fusion protein is flanked with a 6×Histidine tag, a commonly used molecule for pulldown of proteins. Though the inventors have initially kept the histidine tag, it can be easily removed by the addition of a protease cleavage site to the sequence before the tag, which can then be cleaved chemically with the corresponding protease. The fusion protein was recombinantly produced in E. coli, but may be produced in mammalian cells or cell-free in vitro transcription and translation systems. The fusion protein was purified initially with a Nickel resin column that binds the 6×Histidine tag and can be further purified, if necessary, with HPLC.

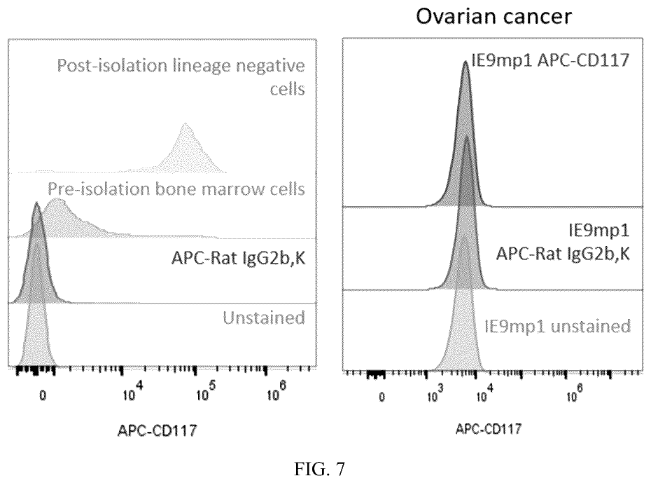

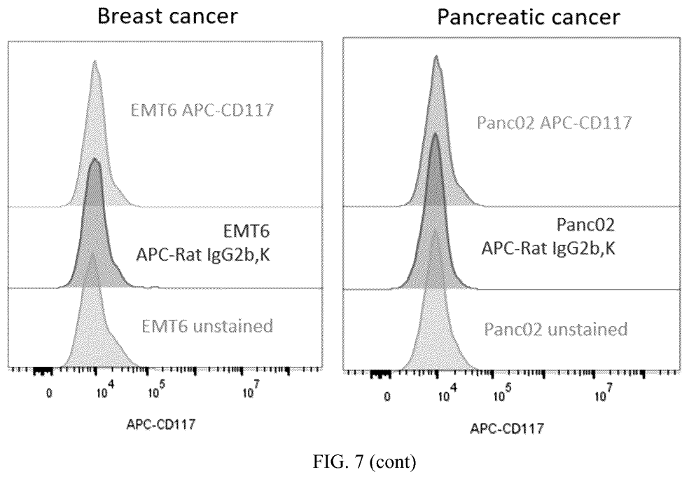

First, to check that there were cancer cell lines negative for CD117, the inventors used flow cytometry on several cell lines. Hematopoietic stem and progenitor cells showed high levels of CD117 expression whereas ovarian cancer cell line IE9mp1, breast cancer cell line EMT6, and pancreatic cancer cell line Panc02 showed no CD117 expression, making CD117 a good candidate for targeting hematopoietic stem and progenitor cells (FIG. 7).

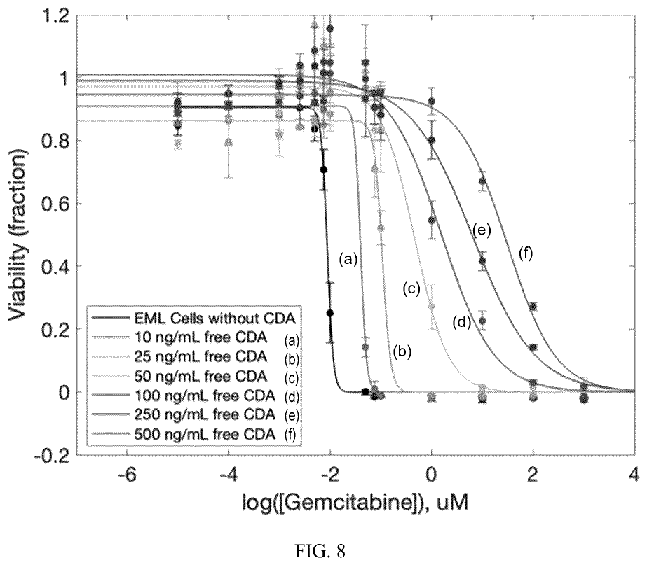

Next, the inventors tested for whether cytidine deaminase can inactivate gemcitabine extracellularly. Cell viabilities of EML cells treated with various concentrations of free CDA were measured by MTS assay, which showed increased IC50 of the cells when CDA is added to the extracellular space (FIG. 8). The inventors concluded that extracellular CDA can reduce gemcitabine-mediated cell killing and protection against gemcitabine is extracellular CDA dose-dependent. This indicated that when the fusion protein with CDA is in the extracellular space, it may be able to inactivate gemcitabine without the need to be internalized.

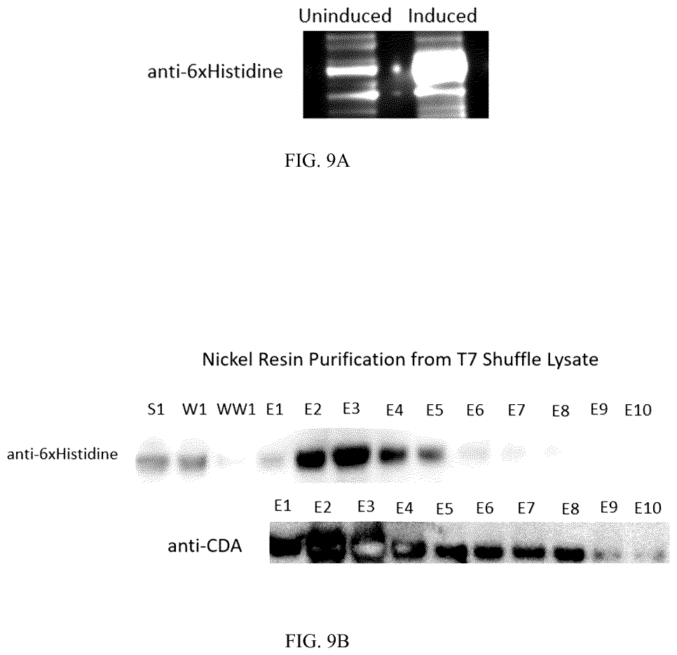

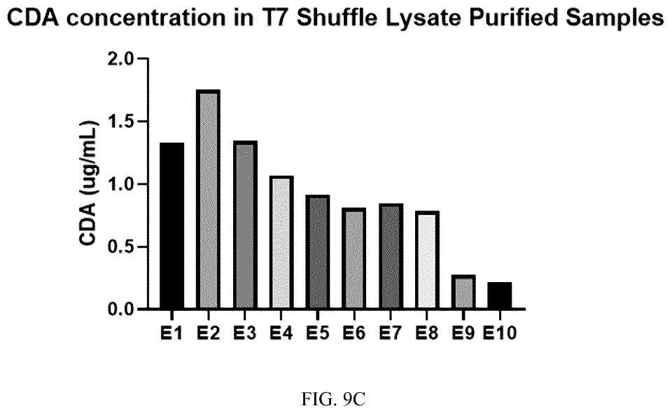

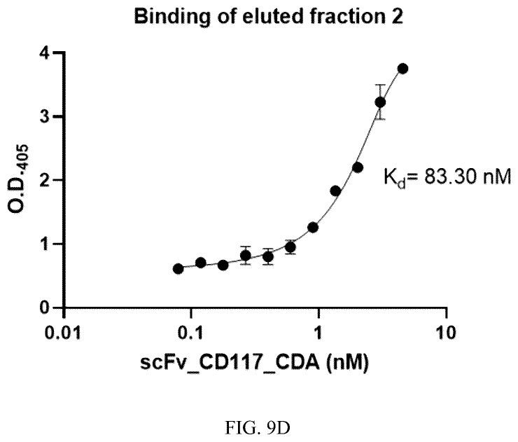

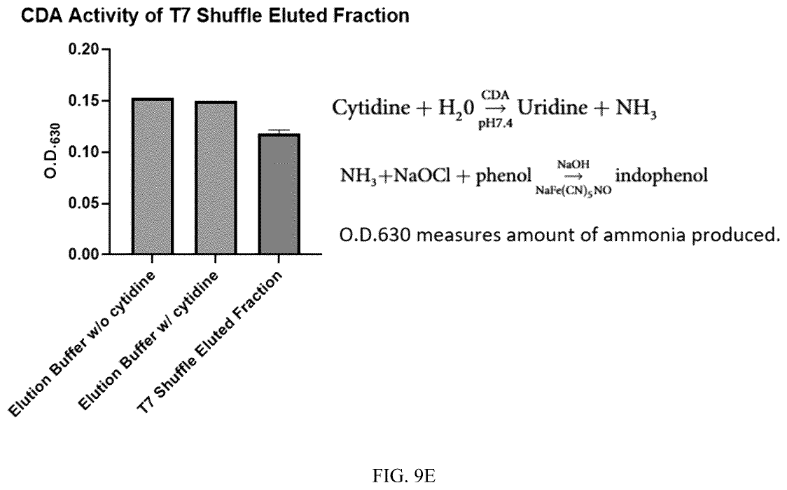

To further characterize the fusion protein, the inventors confirmed that the purified sample from T7 Shuttle bacteria showed good yield of the fusion protein (FIGS. 9A-9C) then characterized the fusion protein for its binding to CD117 by using indirect ELISA to produce a Kd value (FIG. 9D). During the purification, minimal fusion protein was lost in the wash steps and most of the protein was obtained in the elution steps (FIG. 9B). The fusion protein obtained from the purification was ˜1.5 μg/mL (FIG. 9C). For the binding of the fusion protein to the CD117, the affinity may be improved by testing different scFv sequences. Furthermore, the CDA activity of T7 Shuttle eluted fraction was tested by measuring the O.D.₆₃₀, which indicates the production amount of ammonia (FIG. 9E).

### Example 3

In this example, the inventors designed and produced a fusion protein including the single chain variable fragment (scFv) (scFv sequence from WO2020219775A1) of human CD117 antibody with the cytidine deaminase (CDA) enzyme. A 6×Histidine tag followed the fusion protein for easier purification. The contructed protein was produced in E. coli, specifically T7 Shuffle cells, then purified using a Nickel His-Trap column. In some instances, the E. coli were induced under different conditions, including 30° C. for 4 hours, 25° C. for 24 hours, and 30° C. for 4 hours and 25° C. for 20 hours. Lysate from uninduced E. coli was used as a negative control.

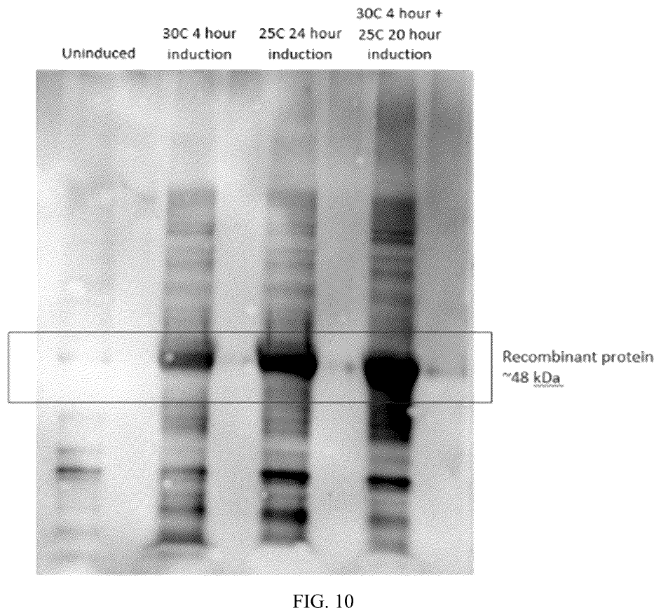

To confirm the production of the fusion protein in T7 Shuffle cells, a Western blot was performed by using an anti-6×Histidine antibody. The fusion protein was expected to have a size between 45-50 kDa. All three induction conditions produced high amounts of the fusion protein, which was detected at 48 kDa (FIG. 10).

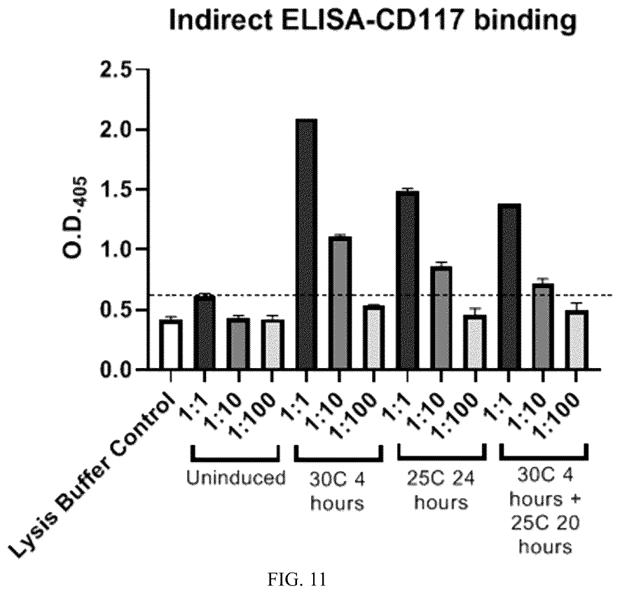

The ability for the fusion protein to bind the target CD117 was also tested. An indirect ELISA against CD117 was used to confirm the binding of the fusion protein to CD117. Uninduced lysate was used as negative control and about 4 times higher signal was observed from the induced lysates (FIG. 11).

### Example 4

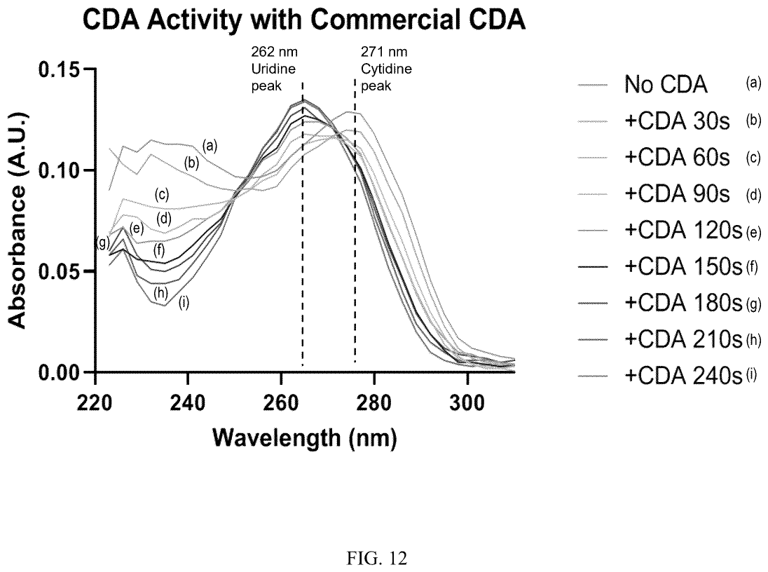

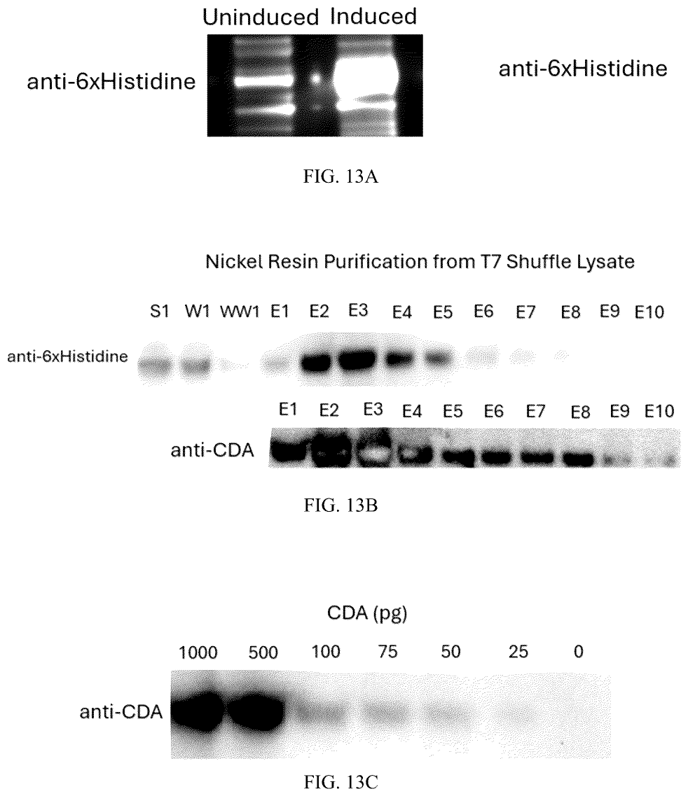

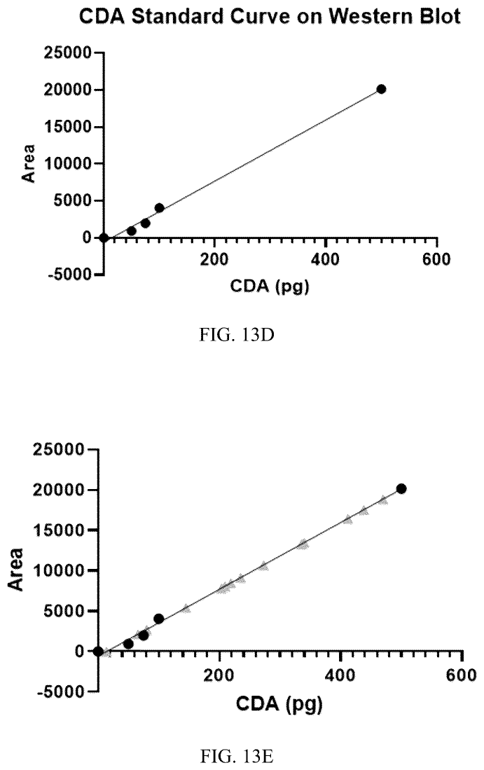

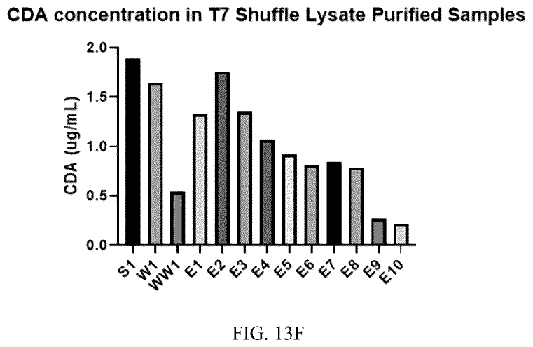

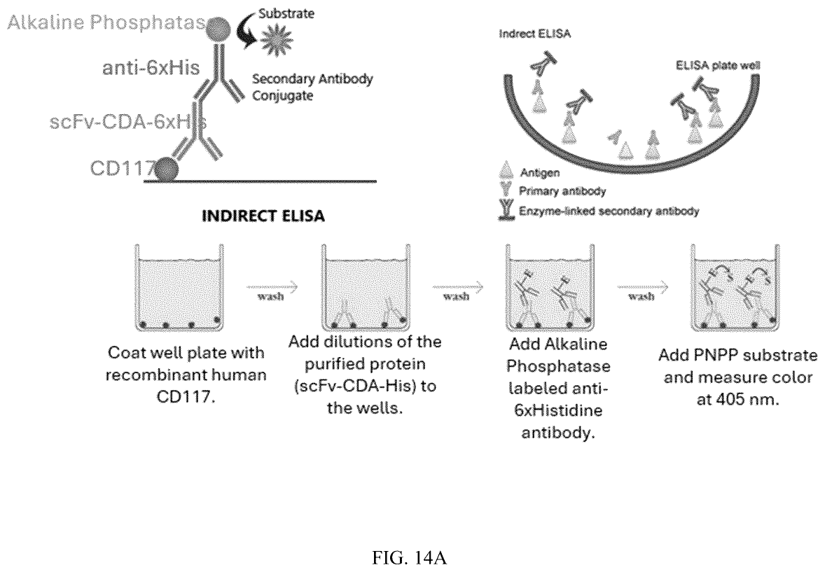

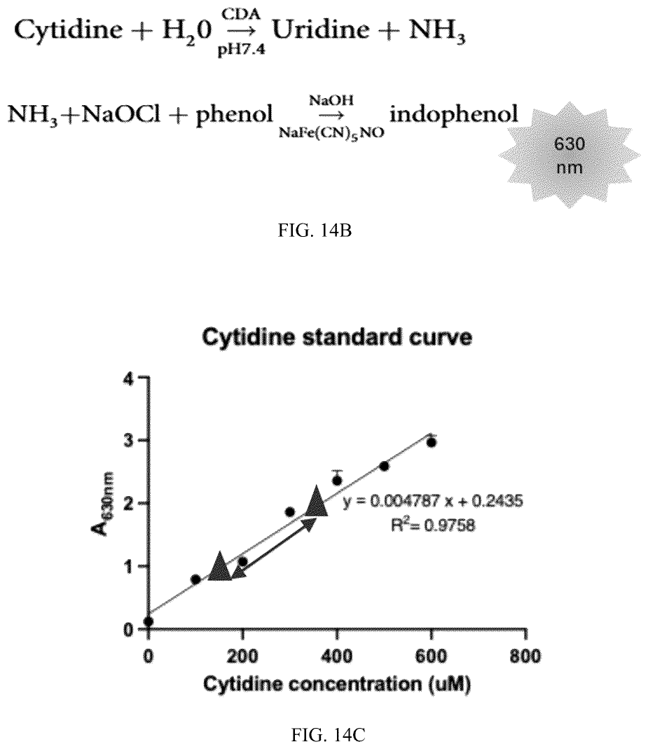

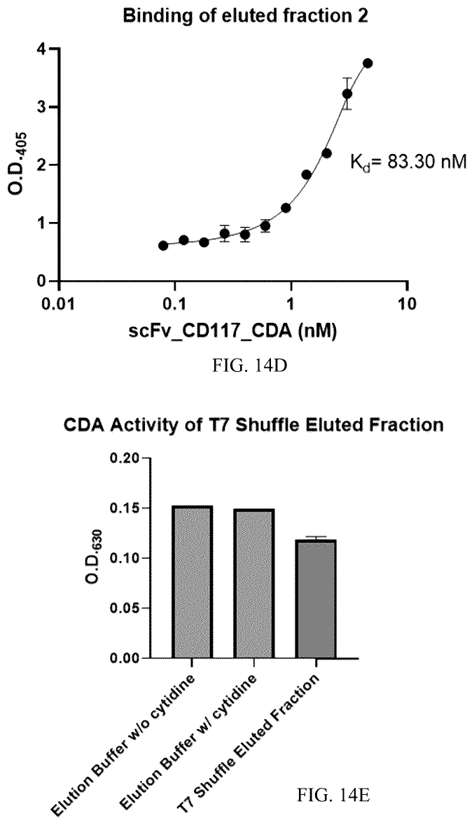

The activity of CDA in a fusion protein may be measured, for example, by evaluating the level of the fusion protein deaminating cytidine to uridine. One method is to observe a shift in the absorbance spectra from 271 nm to 262 nm which are indicative peaks of cytidine to uridine (FIG. 12).

Another method to evaluate the activity of CDA is by measuring the production of ammonia which results from the deamination of cytidine to uridine. The indophenol method for a kinetic assay and produce kcat and Km values from which the catalytic power of the enzyme can be obtained.

### Example 5

The inventors conducted another set of experiments, showing production of three proteins scFv-His, CDA-His and scFv-CDA-His in two strains of E. coli BL21 DE3 and T7 Shuffle cells. Different induction durations, 4 hours, 7 hours and overnight, at different induction temperatures, 30 C and 37 C, were tested. The purpose of these experiments were to identify conditions at which both scFv and CDA are active when produced alone.

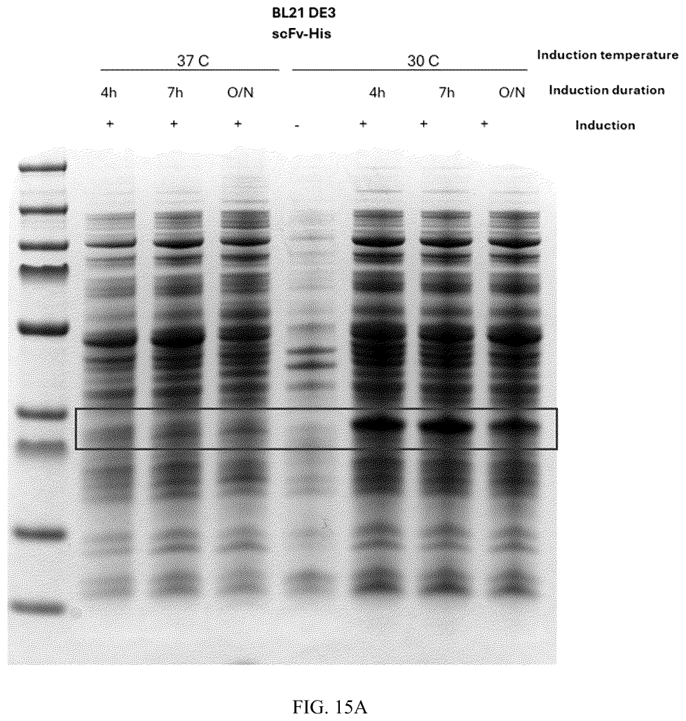

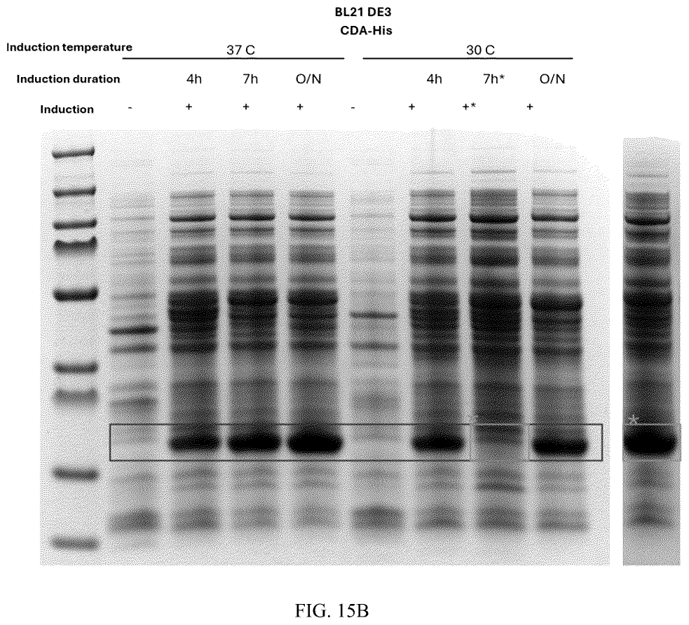

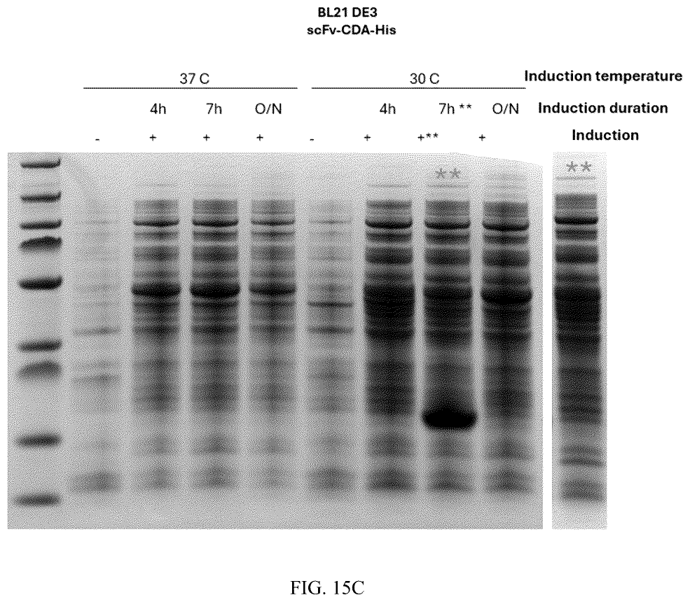

First, the inventors sought to improve recombinant protein production and purity in BL21 DE3 and T7 Shuffle cells. scFv-His, CDA-His, and scFv-CDA-His were induced in the cells for 4 hours, 7 hours, or overnight, at 37° C. or 30° C. (FIGS. 15A-15D). scFv-His was produced at both 37° C. and 30° C. in BL21 DE3, however a higher yield was shown for those induced at 30° C. There was no visible difference between the different induction times (FIG. 15A). CDA-His was produced both at 37° C. and 30° C. in BL21 DE3, with similar yields shown at both temperatures and all induction durations (FIG. 15B). Production of scFv-CDA-His in BL21 DE3 and T7 Shuffle was inconclusive due to lack of unique bands (FIGS. 15C, 15D). scFv-His was produced at 30° C. in T7 Shuffle, with higher yield shown with overnight induction compared to 4 hours of induction. CDA-His is produced at 30° C. in T7 Shuffle, with no visible effect of induction time.

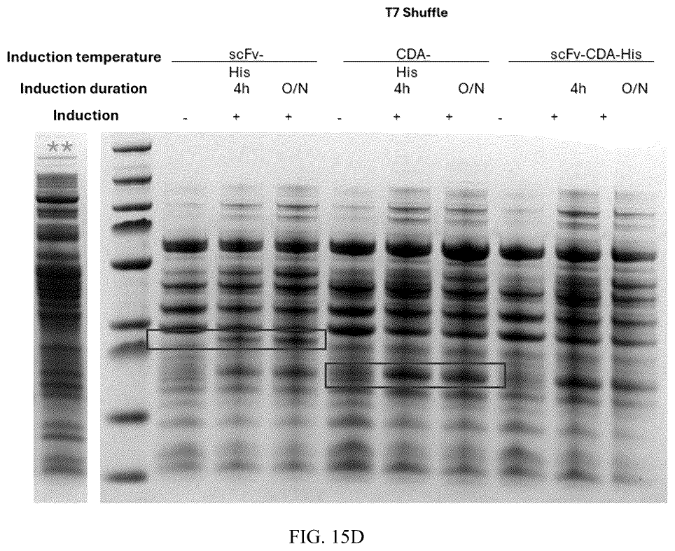

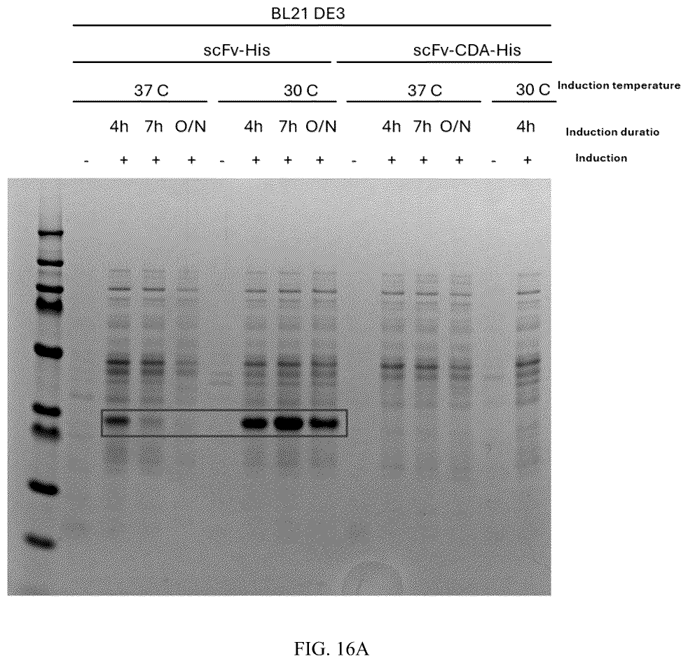

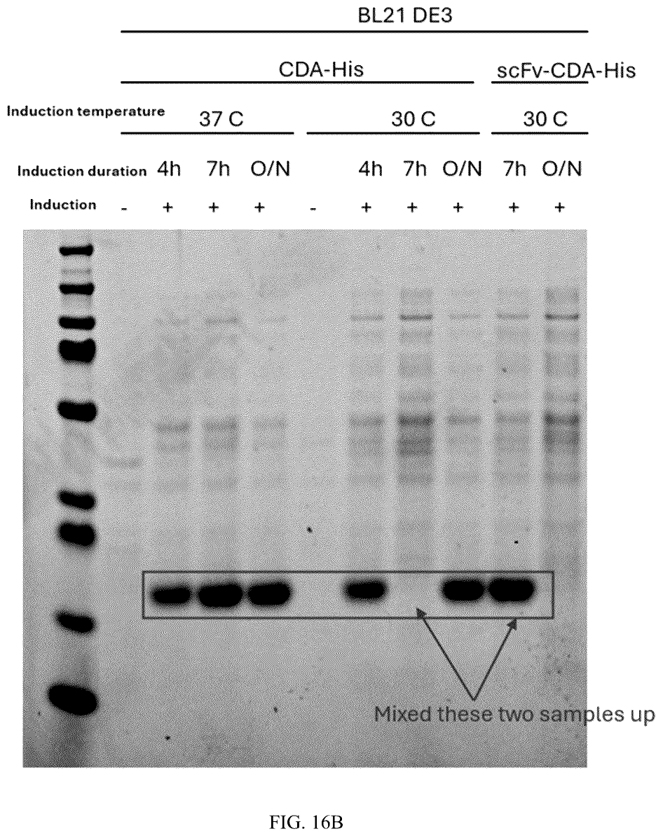

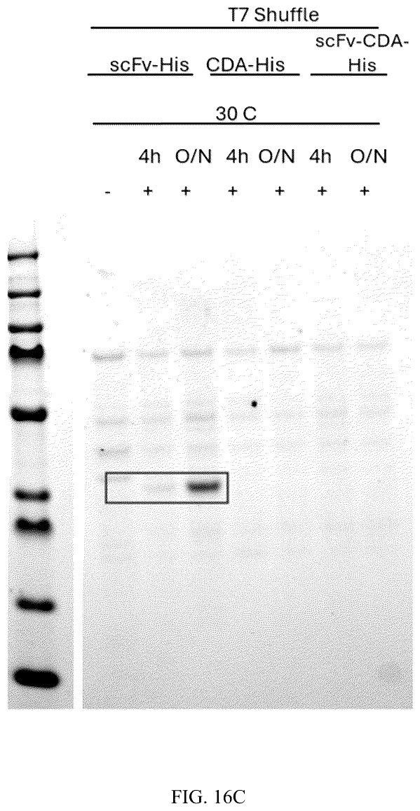

Next, the produced recombinant proteins were purified by using nickel magnetic beads, then the eluted fractions were assessed by using SDS-PAGE (FIGS. 16A-16C). The purification yielded high scFv-His production in BL21 DE3 at 37° C. only for 4 hours of induction, with minimal yield at 7 hours and no visible yield in the overnight group. However, at 30° C. the scFv-His yield was high at all induction durations. Purification of CDA-His from BL21 DE3 yielded high protein at both 30° C. and 37° C. at all induction durations in BL21 DE3. The purity of samples was low. Unique bands for scFv-CDA-His were not observed. In T7 Shuffle, the purification yielded visible scFv-His production with overnight induction and minimal scFv-His with 4 hours of induction. Purification of samples was low. Unique bands were not observed for scFv-CDA-His.

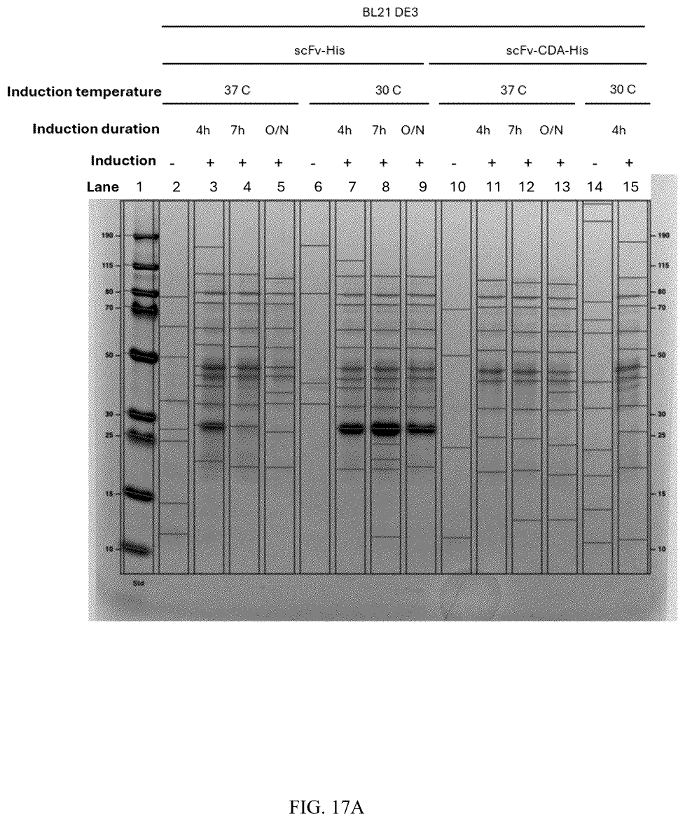

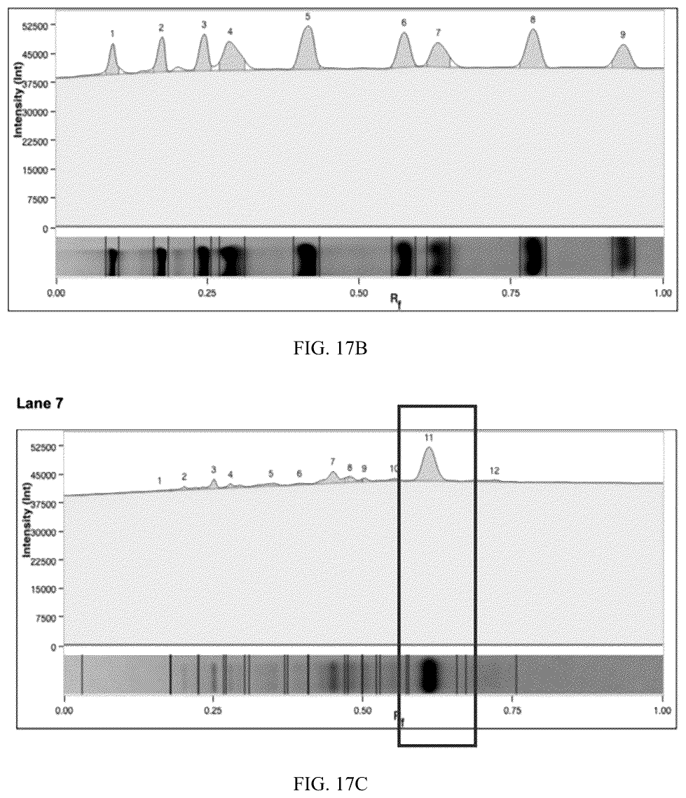

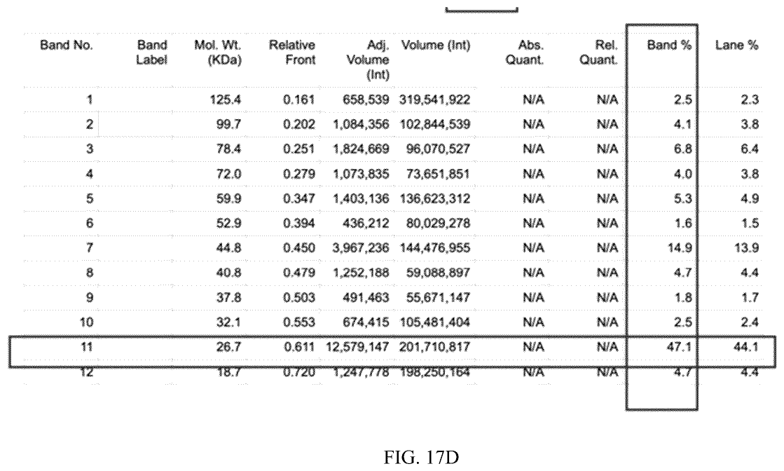

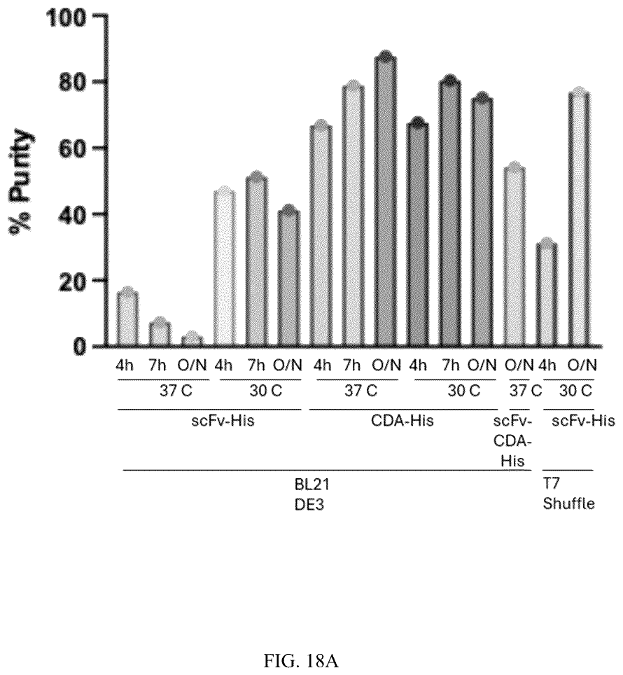

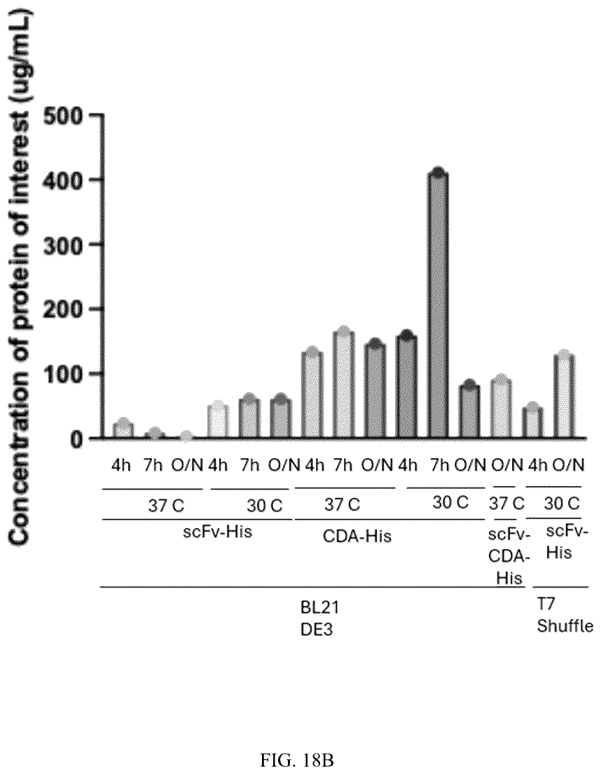

The purity of samples was also analyzed with densitometry on the SDS PAGE gels (FIGS. 17A-17D). Densitometry analysis showed a wide range of purity with the samples ranging from about 5-85%. The amount of protein of interest can be calculated by percent purity multiplied by total protein concentration in the sample measured by Qubit. Concentration of protein of interest shows a high range as well, with the highest yield at about 450 ug/mL and lowest at 4 ug/mL (FIGS. 18A-18B).

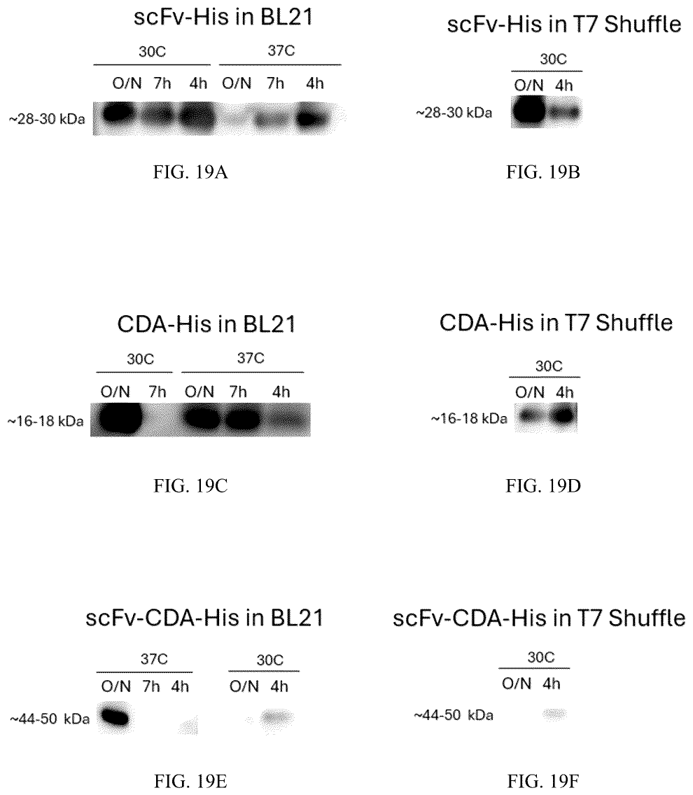

Recombinant protein production was also validated by using Western blot for 6×Histine (FIGS. 19A-19E). Both scFv and CDA were produced in BL21 DE3 and T7 Shuffle, though with different yields at different temperatures and induction times. scFv-CDA showed either none or minimal production in both BL21 and T7 Shuffle strains.

### Example 6

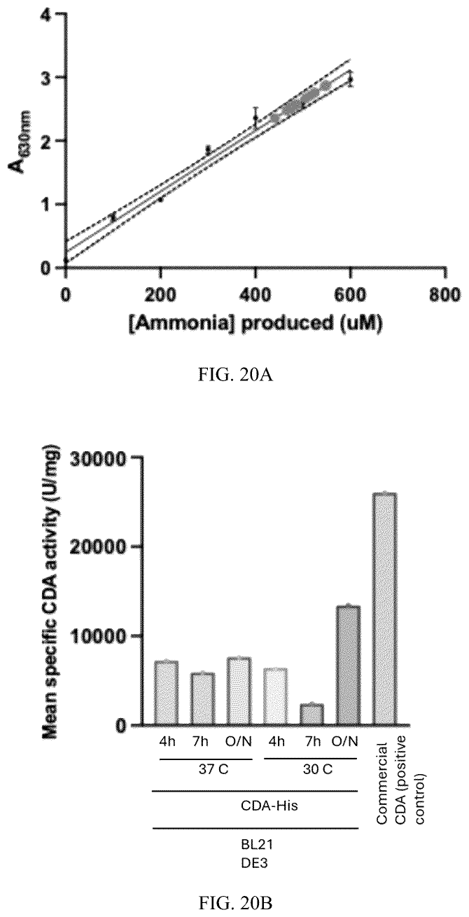

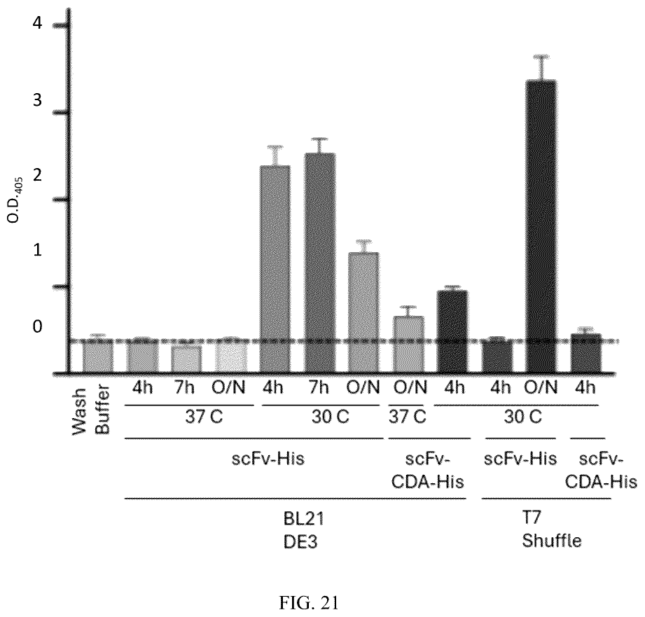

Samples that showed a signal in Western blot and densitometry analysis in Example 5 were assayed for CDA activity and CD117 binding. First, CDA activity was measured by using the indopheol method (FIGS. 20A-20B). The recombinantly produced and purified CDA-His in BL21 DE3 were active. The activity value was approximately half of commercially available human CDA from Abcam®. 1 Unit was the release of 1 μmol ammonia per minute at 37° C., and specific enzyme activity was defined as units of enzyme per mg of enzyme. Next, indirect CD117 ELISA was used to validate the binding of recombinantly produced scFv-His and scFv-CDA-His (FIG. 21). The recombinantly produced and purified scFv-His in BL21 DE3 showed binding to its target CD117 only when produced at 30° C. scFv-CDA-His fusion protein showed binding to CD117 when produced in BL21 DE3 with the given conditions. The scFv-His produced in T7 Shuffle showed binding when induced overnight but not when induced at 4 hours. The signals obtained from alkaline phosphate (shows absorbance at 405 nm) were not normalized to amount of protein; therefore, the intensity of signals were not correlated to binding affinity in FIG. 21.

### Example 7

For every heavy chain and light chain antibody pair, the following protein sequences and their corresponding DNA sequences are created:
1. Light chain variable region of antibody+ (G4S)3 linker+Heavy chain variable region of antibody+Glycine serine linker with variable length (11 possibilities)+CDA+6×His
2. Heavy chain variable region of antibody+ (G4S)3 linker+Light chain variable region of antibody+Glycine serine linker with variable length (11 possibilities)+CDA+6×His
3. CDA+Glycine serine linker with variable length (11 possibilities)+Heavy chain variable region of antibody+ (G4S)3 linker+Light chain variable region of antibody+6×His
4. CDA+Glycine serine linker with variable length (11 possibilities)+Light chain variable region of antibody+ (G4S)3 linker+Heavy chain variable region of antibody+6×His
5. Light chain variable region of antibody+ (G4S)3 linker+Heavy chain variable region of antibody+Glycine serine linker with variable length (11 possibilities)+CDA
6. Heavy chain variable region of antibody+ (G4S)3 linker+Light chain variable region of antibody+Glycine serine linker with variable length (11 possibilities)+CDA
7. CDA+Glycine serine linker with variable length (11 possibilities)+Heavy chain variable region of antibody+ (G4S)3 linker+Light chain variable region of antibody
8. CDA+Glycine serine linker with variable length (11 possibilities)+Light chain variable region of antibody+ (G4S)3 linker+Heavy chain variable region of antibody

In the above, the sequences 5-8 correspond to sequences 1˜4 where the His tag has been removed with a protease e.g. bovine carboxy peptidase A.

It should be noted that even though the peptide sequences will remain the same, the DNA sequences can vary slightly based on codon optimization for E. coli.

The CDA gene sequence can be obtained with the National Library of Medicine accession number P32320. The antibody sequences were obtained from International Publication No. WO 2020/219775 A1.

All sequences were expressed in a pET28a(+) plasmid following the ribosome binding site, however, they can be expressed in any plasmid with a T7 promoter and is compatible with expression in BL21 DE3 and T7 Shuffle strains of E. coli.

TABLE 1

| Naming in | | | |
|---|---|---|---|
| Sequence_ | | | |
| Name | Description | Peptide_sequence | DNA sequence |
| Ab58_LC | Ab58_light_chain_ | DIQMTQSPSSVSASVGDRVTITCR | GACATCCAGATGACCCAGTCTCCATCTTCCGTGTCTGCATCTGT |
| | variable_region | ASQGISSWLAWYQQKPGKAPKLL | AGGAGACAGAGTCACCATCACTTGTCGGGCGAGTCAGGGTAT |
| | | IIYAASSLQSGVPSRFSGSGSGTD | TAGCAGCTGGTTAGCCTGGTATCAGCAGAAACCAGGGAAAGC |
| | | FTLTISSLQPEDFATYYCQQTNSF | CCCTAAGCTCCTGATCTATGCTGCATCCAGTTTGCAAAGTGGG |
| | | PYTFGGGTKVEIK (SEQ ID NO: 441) | GTCCCATCAAGGTTCAGCGGCAGTGGATCTGGGACAGATTTCA |
| | | | CTCTCACCATCAGCAGCCTGCAGCCTGAAGATTTTGCAACTTA |
| | | | TTACTGTCAGCAAACAAATAGTTTCCCTTACACTTTTGGCGGA |
| | | | GGGACCAAGGTTGAGATCAAA (SEQ ID NO: 451) |
| Ab58_HC | Ab58_heavy_chain_ | EVQLLESGGGLVQPGGSLRLSCA | GAGGTGCAGCTGTTGGAGTCTGGGGGAGGCTTGGTACAGCCT |
| | variable_region | ASGETFSNYAMSWVRQAPGKGLE | GGGGGGTCCCTGAGACTCTCCTGTGCAGCCTCTGGATTCACCT |
| | | WVSAISGSGGSTYYADSVKGRFTI | TTAGCAATTATGCCATGAGCTGGGTCCGCCAGGCTCCAGGGAA |
| | | SRDNSKNTLYLQMNSLRAEDTAV | GGGGCTGGAGTGGGTCTCAGCTATTAGTGGTAGTGGTGGTAGC |
| | | YYCAKGPPTYHTNYYYMDVWGK | ACATACTACGCAGACTCCGTGAAGGGCCGGTTCACCATCTCCA |
| | | GTTVTVSS (SEQ ID NO: 442) | GAGACAATTCCAAGAACACGCTGTATCTGCAAATGAACAGCC |
| | | | TGAGAGCCGAGGACACGGCGGTGTACTACTGCGCCAAGGGCC |
| | | | CTCCTACATACCACACAAACTACTACTACATGGACGTATGGGG |
| | | | CAAGGGTACAACTGTCACCGTCTCCTCA (SEQ ID NO: 452) |
| Ab61_LC | Ab61_light_chain_ | DIQMTQSPSSVSASVGDRVTITCR | GACATCCAGATGACCCAGTCTCCATCTTCCGTGTCTGCATCTGT |
| | variable_region | ASQGISSWLAWYQQKPGKAPKLL | AGGAGACAGAGTCACCATCACTTGTCGGGCGAGTCAGGGTAT |
| | | IYAASSLQQSGVPSRFSGSGSGTD | TAGCAGCTGGTTAGCCTGGTATCAGCAGAAACCAGGGAAAGC |
| | | FTLTISSLQPEDFATYYCQQTNSE | CCCTAAGCTCCTGATCTATGCTGCATCCAGTTTGCAAAGTGGG |
| | | PYTFGGGTKVEIK (SEQ ID NO: 443) | GTCCCATCAAGGTTCAGCGGCAGTGGATCTGGGACAGATTTCA |
| | | | CTCTCACCATCAGCAGCCTGCAGCCTGAAGATTTTGCAACTTA |
| | | | TTACTGTCAGCAAACAAATAGTTTCCCTTACACTTTTGGCGGA |
| | | | GGGACCAAGGTTGAGATCAAA (SEQ ID NO: 453) |
| Ab61_HC | Ab61_heavy_chain_ | EVQLLESGGGLVQPGGSLRLSCA | GAGGTGCAGCTGTTGGAGTCTGGGGGAGGCTTGGTACAGCCT |
| | variable_region | ASGETESSYVMIWVRQAPGKGLEWV | GGGGGGTCCCTGAGACTCTCCTGTGCAGCCTCTGGATTCACCT |
| | | SSISGDSVTTYYADSVKGRFTISRDNS | TTAGCAGCTATGTCATGATCTGGGTCCGCCAGGCTCCAGGGAA |
| | | KNTLYLQMNSLRAEDTAVYYCAKGP | GGGGCTGGAGTGGGTCTCAAGCATTAGTGGTGACAGCGTAAC |
| | | PTYHTNYYYMDVWGKGTTVTVSS | AACATACTACGCAGACTCCGTGAAGGGCCGGTTCACCATCTCC |
| | | (SEQ ID NO: 444) | AGAGACAATTCCAAGAACACGCTGTATCTGCAAATGAACAGC |
| | | | CTGAGAGCCGAGGACACGGCGGTGTACTACTGCGCCAAGGGC |
| | | | CCTCCTACATACCACACAAACTACTACTACATGGACGTATGGG |
| | | | GCAAGGGTACAACTGTCACCGTCTCCTCA (SEQ ID NO: 454) |
| Ab66_LC | Ab66_light_chain_ | DIQMTQSPSSLSASVGDRVTITCRASQ | GACATCCAGATGACCCAGTCTCCATCCTCCCTGTCTGCATCTGT |
| | variable_region | SISSYLNWYQQKPGKAPKLLIYAASSL | AGGAGACAGAGTCACCATCACTTGCCGGGCAAGTCAGAGCAT |
| | | QSGVPSRFSGSGSGTDFTLTISSLQPED | TAGCAGCTATTTAAATTGGTATCAGCAGAAACCAGGGAAAGC |
| | | FATYYCQQSYIAPYTFGGGTKVEIK | CCCTAAGCTCCTGATCTATGCTGCATCCAGTTTGCAAAGTGGG |
| | | (SEQ ID NO: 445) | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTACTGTCAGCAAAGCTACATCGCCCCTTACACTTTTGGCGGA |
| | | | GGGACCAAGGTTGAGATCAAA (SEQ ID NO: 455) |
| Ab66_HC | Ab66_heavy_chain_ | EVQLVESGGGLVQPGGSLRLSCA | GAGGTGCAGCTGGTGGAGTCTGGGGGAGGCTTGGTCCAGCCT |
| | variable_region | ASGETFSDHYMDWVRQAPGKGLASG | GGAGGGTCCCTGAGACTCTCCTGTGCAGCCTCTGGATTCACCT |
| | | ETFSDHYMDWVRQAPGKGLEWVGRI | TCAGTGACCACTACATGGACTGGGTCCGCCAGGCTCCAGGGA |
| | | RNKASSYTTEYAASVKGRFTISRDDS | AGGGGCTGGAGTGGGTTGGCCGTACTAGAAACAAAGCTAGTA |
| | | KNSLYLQMNSLKTEDTAVYYCAREP | GTTACACCACAGAATACGCCGCGTCTGTGAAAGGCAGATTCAC |
| | | KYWIDFDLWGRGTLVTVSS (SEQ ID | CATCTCAAGAGATGATTCAAAGAACTCACTGTATCTGCAAATG |
| | | NO: 446) | AACAGCCTGAAAACCGAGGACACGGCGGTGTACTACTGCGCC |
| | | | AGAGAGCCTAAATACTGGATCGACTTCGACCTATGGGGGAGA |
| | | | GGTACCTTGGTCACCGTCTCCTCA (SEQ ID NO: 456) |
| Ab68_LC | Ab68_light_chain_ | DIQMTQSPSSLSASVGDRVTITCRASQ | GACATCCAGATGACCCAGTCTCCATCCTCCCTGTCTGCATCTGT |
| | variable_region | SISSYLNWYQQKPGKAPKLLIYAASSL | AGGAGACAGAGTCACCATCACTTGCCGGGCAAGTCAGAGCAT |
| | | QSGVPSRFSGSGSGTDFTLTISSLQPED | TAGCAGCTATTTAAATTGGTATCAGCAGAAACCAGGGAAAGC |
| | | FATYYCQQSYIAPYTFGGGTKVEIK | CCCTAAGCTCCTGATCTATGCTGCATCCAGTTTGCAAAGTGGG |
| | | (SEQ ID NO: 447) | GTCCCATCAAGGTTCAGTGGCAGTGGATCTGGGACAGATTTCA |
| | | | CTCTCACCATCAGCAGTCTGCAACCTGAAGATTTTGCAACTTA |
| | | | CTACTGTCAGCAAAGCTACATCGCCCCTTACACTTTTGGCGGA |
| | | | GGGACCAAGGTTGAGATCAAA (SEQ ID NO: 457) |
| Ab68_HC | Ab68_heavy_chain_ | EVQLVESGGGLVQPGRSLRLSCTASG | GAGGTGCAGCTGGTGGAGTCTGGGGGAGGCTTGGTACAGCCA |
| | variable_region | FTFSDHDMNWVRQAPGKGLEWVGR | GGGCGGTCCCTGAGACTCTCCTGTACAGCTTCTGGATTCACCT |
| | | TRNAAGSYTTEYAASVKGRFTISRDD | TCAGTGACCACGACATGAACTGGGTCCGCCAGGCTCCAGGGA |
| | | SKNSLYLQMNSLKTEDTAVYYCARE | AGGGGCTGGAGTGGGTTGGCCGTACTAGAAACGCCGCTGGAA |
| | | PKYWIDFDLWGRGTLVTSS (SEQ ID | GTTACACCACAGAATACGCCGCGTCTGTGAAAGGCAGATTCAC |
| | | NO: 448) | CATCTCAAGAGATGATTCAAAGAACTCACTGTATCTGCAAATG |
| | | | AACAGCCTGAAAACCGAGGACACGGCGGTGTACTACTGCGCC |
| | | | AGAGAGCCTAAATACTGGATCGACTTCGACCTATGGGGG (SEQ |
| | | | ID NO: 458) |
| Ab69 HC | Ab69_heavy_chain_ | EVQLVESGGGLVQPGGSLRLSCA | GAGGTGCAGCTGGTGGAGTCTGGGGGAGGCTTGGTCCAGCCT |
| | variable_region | ASGETFVDHDMDWVRQAPGKGLEW | GGAGGGTCCCTGAGACTCTCCTGTGCAGCCTCTGGATTCACCT |
| | | VGRTRNKLGSYTTEYAASVKGRET | TCGTAGACCACGACATGGACTGGGTCCGCCAGGCTCCAGGGA |
| | | GRDDSKNSLYLQMNSLKTEDTAVYY | AGGGGCTGGAGTGGGTTGGCCGTACTAGAAACAAACTAGGAA |
| | | CAREPKYWIDFDLWGRGTLVTVSS | GTTACACCACAGAATACGCCGCGTCTGTGAAAGGCAGATTCAC |
| | | (SEQ ID NO: 449) | CATCTCAAGAGATGATTCAAAGAACTCACTGTATCTGCAAATG |
| | | | AACAGCCTGAAAACCGAGGACACGGCGGTGTACTACTGCGCC |
| | | | AGAGAGCCTAAATACTGGATCGACTTCGACCTATGGGGGAGA |
| | | | GGTACCTTGGTCACCGTCTCCTCA (SEQ ID NO: 459) |
| Ab69_LC | Ab69_light_chain_ | DIQMTQSPSSLSASVGDRVTITCR | GACATCCAGATGACCCAGTCTCCATCCTCCCTGTCTGCATCTGT |
| | variable_region | ASQSISSYLNWYQQKPGKAPKLLIYA | AGGAGACAGAGTCACCATCACTTGCCGGGCAAGTCAGAGCAT |
| | | ASSLQSGVPSRFSGSGSGTDFTLTISSL | TAGCAGCTATTTAAATTGGTATCAGCAGAAACCAGGGAAAGC |
| | | QPEDFATYYCQQSYIAPYIFGGGTKV | CCCTAAGCTCCTGATCTATGCTGCATCCAGTTTGCAAAGTGGG |
| | | EIK (SEQ ID NO: 450) | GTCCCATCAAGGTTCAGTGGCAGTGGATGCGGTACAGATTTCA |
| | | | CTCTCACCATCAGCAGTCTGCAACCTGAAGATTTTGCAACTTA |
| | | | CTACTGTCAGCAAAGCTACATCGCCCCTTACACTTTTGGCGGA |
| | | | GGGACCAAGGTTGAGATCAAA (SEQ ID NO: 460) |

TABLE 2
List of potential linkers betweeen the scFv and CDA

| Naming in | | | |
|---|---|---|---|
| Sequence_ | | | |
| Name | Description | Peptide_sequence | DNA sequence |
| G4S linker | G4S linker | GGGGS (SEQ ID NO: 461) | ggtggcgggggatca (SEQ ID NO: 472) |
| (G4S)3 | (G4S)3 linker | GGGGSGGGGSGGGGS (SEQ ID NO: | ggagggggcggatcaggcggaggtggatcgggagggggtggtagc (SEQ ID NO: 473) |
| linker | | 462) | |
| (G4S)4 | (G4S)4 linker | GGGGSGGGGSGGGGSGGGGS (SEQ | ggtggaggcggttcgggtggagggggatctggtggcggaggtagcggtggcgggggatca (SEQ |
| linker | | ID NO: 463) | ID NO: 474) |
| (G4S)5 | (G4S)5 linker | GGGGSGGGGSGGGGSGGGGSGGGGS | ggtggaggcggttcgggtggagggggatctggtggcggaggtagcggtggcgggggatcaggtgg |
| linker | | (SEQ ID NO: 464) | cgggggatca (SEQ ID NO: 475) |
| (G4S)6 | (G4S)6 linker | GGGGSGGGGSGGGGSGGGGSGGGGS | ggtggaggcggttcgggtggagggggatctggtggcggaggtagcggtggcgggggatcaggtgg |
| linker | | GGGGS (SEQ ID NO: 465) | cgggggatcaggtggcgggggatca (SEQ ID NO: 476) |
| (G4S)7 | (G4S)7 linker | GGGGSGGGGSGGGGSGGGGSGGGGS | ggtggaggcggttcgggtggagggggatctggtggcggaggtagcggtggcgggggatcaggtgg |
| linker | | GGGGSGGGGS (SEQ ID NO: 466) | cgggggatcaggtggcgggggatcaggtggcgggggatca (SEQ ID NO: 477) |
| (G4S)8 | (G4S)8 linker | GGGGSGGGGSGGGGSGGGGSGGGGS | ggtggaggcggttcgggtggagggggatctggtggcggaggtagcggtggcgggggatcaggtgg |
| linker | | GGGGSGGGGSGGGGS (SEQ ID NO: | cgggggatcaggtggcgggggatcaggtggcgggggatcaggtggcgggggatca (SEQ ID |
| | | 467) | NO: 478) |
| (G4S)9 | (G4S)9 linker | GGGGSGGGGSGGGGSGGGGSGGGGS | ggtggaggcggttcgggtggagggggatctggtggcggaggtagcggtggcgggggatcaggtgg |
| linker | | GGGGSGGGGSGGGGSGGGGS (SEQ | cgggggatcaggtggcgggggatcaggtggcgggggatcaggtggcgggggatcaggtggcggg |
| | | ID NO: 468) | ggatca (SEQ ID NO: 479) |
| (G4S)10 | (G4S)10 linker | GGGGSGGGGSGGGGSGGGGSGGGGS | ggtggaggcggttcgggtggagggggatctggtggcggaggtagcggtggcgggggatcaggtgg |
| linker | | GGGGSGGGGSGGGGSGGGGSGGGGS | cgggggatcaggtggcgggggatcaggtggcgggggatcaggtggcgggggatcaggtggcggg |
| | | (SEQ ID NO: 469) | ggatcaggtggcgggggatca (SEQ ID NO: 480) |
| (G4S)11 | (G4S)11 linker | GGGGSGGGGSGGGGSGGGGSGGGGS | ggtggaggcggttcgggtggagggggatctggtggcggaggtagcggtggcgggggatcaggtgg |
| linker | | GGGGSGGGGSGGGGSGGGGSGGGGS | cgggggatcaggtggcgggggatcaggtggcgggggatcaggtggcgggggatcaggtggcggg |
| | | GGGGS (SEQ ID NO: 470) | ggatcaggtggcgggggatcaggtggcgggggatca (SEQ ID NO: 481) |
| (G4S)112 | (G4S)112 linker | GGGGSGGGGSGGGGSGGGGSGGGGS | ggtggaggcggttcgggtggagggggatctggtggcggaggtagcggtggcgggggatcaggtgg |
| linker | | GGGGSGGGGSGGGGSGGGGSGGGGS | gggggatcaggtggcgggggatcaggtggcgggggatcaggtggcgggggatcaggtggcggg |
| | | GGGGSGGGGS (SEQ ID NO: 471) | ggatcaggtggcgggggatcaggtggcgggggatcaggtggcgggggatca (SEQ ID NO: |
| | | | 482) |

TABLE 3
Human CDA sequence

| Naming | | |
|---|---|---|
| in | | |
| Sequence_ | | |
| Name | Peptide_sequence | DNA sequence |
| CDA | MAQKRPACTLKPECVQQLLV | cccgctgctctgctgcctgcccggggtaccaacatggcccagaagcgtcctgcct |
| | CSQEAKKSAYCPYSHFPVGA | gcaccctgaagcctgagtgtgtccagcagctgctggtttgctcccaggaggccaa |
| | ALLTQEGRIFKGCNIENACYP | gaagtcagcctactgcccctacagtcactttcctgtgggggctgccctgctcaccc |
| | LGICAERTAIQKAVSEGYKDF | aggaggggagaatcttcaaagggtgcaacatagaaaatgcctgctacccgctgg |
| | RAIAIASDMQDDFISPCGACR | gcatctgtgctgaacggaccgctatccagaaggccgtctcagaagggtacaagg |
| | QVMREFGTNWPVYMTKPDG | atttcagggcaattgctatcgccagtgacatgcaagatgattttatctctccatgtgg |
| | TYIVMTVQELLPSSFGPEDLQ | ggcctgcaggcaagtcatgagagagtttggcaccaactggcccgtgtacatgacc |
| | KTQ (SEQ ID NO: 483) | aagcoggatggtacgtatatt (SEQ ID NO: 484) |

TABLE 4
Purification His tag

| Naming in | | Peptide_ | |
|---|---|---|---|
| Sequence_Name | Description | sequence | DNA sequence |
| HIS | 6xHis | HHHHHH (SEQ ID NO: | caccatcatcatcaccat (SEQ ID NO: 486) |
| | | 485) | |

TABLE 5

| Sequence_ | |
|---|---|
| Name | Protein_Sequence |
| Ab58_LC_ | DIQMTQSPSSVSASVGDRVTITCRASQGISSWLAWYQQKPGKAPKLLLIYAASSLQ |
| (G4S)3_HC_G4S | SGVPSRFSGSGSGTDFTLTISSLQPEDFATYYCQQTNSFPYTFGGGTKVEIKGGGGS |
| linker_CDA_ | GGGGSGGGGSEVQLLESGGGLVQPGGSLRLSCAASGETFSNYAMSWVRQAPGKG |
| HIS | LEWVSAISGSGGSTYYADSVKGRFTISRDNSKNTLYLQMNSLRAEDTAVYYCAKG |
| | PPTYHTNYYYMDVWGKGTTVTVSSGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQHHHHHH (SEQ ID NO: 1) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)3 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQHHHHHH (SEQ ID NO: 2) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)4 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQHHHHHH (SEQ ID NO: 3) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)5 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 4) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)6 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQHHHHHH (SEQ ID NO: 5) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)7 | GGGGGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQHHHHHH (SEQ ID |
| | NO: 6) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)8 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 7) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)9 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQHHHH |
| | HH (SEQ ID NO: 8) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)10 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | QHHHHHH (SEQ ID NO: 9) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)11 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQHHHHHH (SEQ ID NO: 10) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)112 | GGGGSGGGGSEVQLLESGGGLVQPGGSLRLSCAASGETFSNYAMSWVRQAPGKG |
| linker_CDA_ | LEWVSAISGSGGSTYYADSVKGRFTISRDNSKNTLYLQMNSLRAEDTAVYYCAKG |
| HIS | PPTYHTNYYYMDVWGKGTTVTVSSGGGGSGGGGSGGGGSGGGGSGGGGSGGGG |
| | SGGGGSGGGGSGGGGSGGGGSGGGGSGGGGSMAQKRPACTLKPECVQQLLVCSQ |
| | EAKKSAYCPYSHFPVGAALLTQEGRIFKGCNIENACYPLGICAERTAIQKAVSEGY |
| | KDFRAIAIASDMQDDFISPCGACRQVMREFGTNWPVYMTKPDGTYIVMTVQELLP |
| | SSFGPEDLQKTQHHHHHH (SEQ ID NO: 11) |
| Ab61_LC_ | DIQMTQSPSSVSASVGDRVTITCRASQGISSWLAWYQQKPGKAPKLLIYAASSLQQ |
| (G4S)3_HC_G4S | SGVPSRFSGSGSGTDFTLTISSLQPEDFATYYCQQTNSEPYTFGGGTKVEIKGGGGS |
| linker_CDA_ | GGGGSGGGGSEVQLLESGGGLVQPGGSLRLSCAASGETESSYVMIWVRQAPGKGL |
| HIS | EWVSSISGDSVTTYYADSVKGRFTISRDNSKNTLYLQMNSLRAEDTAVYYCAKGP |
| | PTYHTNYYYMDVWGKGTTVTVSSGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQHHHHHH (SEQ ID NO: 12) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)3 | GGGGGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQHHHHHH (SEQ ID NO: 13) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)4 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQHHHHHH (SEQ ID NO: 14) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)5 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 15) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)6 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | MAQKRPACTLKPECVQQLLVCSQEAKKSAYCPYSHFPVGAALLTQEGRIFKGCNIE |
| | NACYPLGICAERTAIQKAVSEGYKDFRAIAIASDMQDDFISPCGACRQVMREFGTN |
| | WPVYMTKPDGTYIVMTVQELLPSSFGPEDLQKTQHHHHHH (SEQ ID NO: 16) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)7 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | GGGGSMAQKRPACTLKPECVQQLLVCSQEAKKSAYCPYSHFPVGAALLTQEGRIF |
| | KGCNIENACYPLGICAERTAIQKAVSEGYKDFRAIAIASDMQDDFISPCGACRQVM |
| | REFGTNWPVYMTKPDGTYIVMTVQELLPSSFGPEDLQKTQHHHHHH (SEQ ID NO: |
| | 17) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)8 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | GGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 18) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)9 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | GGGGSGGGGSGGGGSMAQKRPACTLKPECVQQLLVCSQEAKKSAYCPYSHFPVG |
| | AALLTQEGRIFKGCNIENACYPLGICAERTAIQKAVSEGYKDFRAIAIASDMQDDFI |
| | SPCGACRQVMREFGTNWPVYMTKPDGTYIVMTVQELLPSSFGPEDLQKTQHHHH |
| | HH (SEQ ID NO: 19) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)10 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | GGGGSGGGGSGGGGSGGGGSMAQKRPACTLKPECVQQLLVCSQEAKKSAYCPYS |
| | HFPVGAALLTQEGRIFKGCNIENACYPLGICAERTAIQKAVSEGYKDFRAIAIASDM |
| | QDDFISPCGACRQVMREFGTNWPVYMTKPDGTYIVMTVQELLPSSFGPEDLQKTQ |
| | HHHHHH (SEQ ID NO: 20) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)11 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | GGGGSGGGGSGGGGSGGGGSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQHHHHHH (SEQ ID NO: 21) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)112 | GGGGSGGGGSEVQLLESGGGLVQPGGSLRLSCAASGETESSYVMIWVRQAPGKGL |
| linker_CDA_ | EWVSSISGDSVTTYYADSVKGRFTISRDNSKNTLYLQMNSLRAEDTAVYYCAKGP |
| HIS | PTYHTNYYYMDVWGKGTTVTVSSGGGGSGGGGSGGGGSGGGGSGGGGSGGGGS |
| | GGGGSGGGGSGGGGSGGGGSGGGGSGGGGSMAQKRPACTLKPECVQQLLVCSQE |
| | AKKSAYCPYSHFPVGAALLTQEGRIFKGCNIENACYPLGICAERTAIQKAVSEGYK |
| | DFRAIAIASDMQDDFISPCGACRQVMREFGTNWPVYMTKPDGTYIVMTVQELLPSS |
| | FGPEDLQKTQHHHHHH (SEQ ID NO: 22) |
| Ab66_LC_ | DIQMTQSPSSLSASVGDRVTITCRASQSISSYLNWYQQKPGKAPKLLIYAASSLQSG |
| (G4S)3_HC_G4S | VPSRFSGSGSGTDFTLTISSLQPEDFATYYCQQSYIAPYTFGGGTKVEIKGGGGSGG |
| linker_CDA_ | GGSGGGGSEVQLVESGGGLVQPGGSLRLSCAASGETFSDHYMDWVRQAPGKGLA |
| HIS | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSMAQKRPA |
| | CTLKPECVQQLLVCSQEAKKSAYCPYSHFPVGAALLTQEGRIFKGCNIENACYPLGI |
| | CAERTAIQKAVSEGYKDFRAIAIASDMQDDFISPCGACRQVMREFGTNWPVYMTK |
| | PDGTYIVMTVQELLPSSFGPEDLQKTQHHHHHH (SEQ ID NO: 23) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)3 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSMAQKRPACTLKPECVQQLLVCSQEAKKSAYCPYSHFPVGAALLTQEGRIFKG |
| | CNIENACYPLGICAERTAIQKAVSEGYKDFRAIAIASDMQDDFISPCGACRQVMREF |
| | GTNWPVYMTKPDGTYIVMTVQELLPSSFGPEDLQKTQHHHHHH (SEQ ID NO: 24) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)4 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSMAQKRPACTLKPECVQQLLVCSQEAKKSAYCPYSHFPVGAALLTQE |
| | GRIFKGCNIENACYPLGICAERTAIQKAVSEGYKDFRAIAIASDMQDDFISPCGACR |
| | QVMREFGTNWPVYMTKPDGTYIVMTVQELLPSSFGPEDLQKTQHHHHHH (SEQ ID |
| | NO: 25) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)5 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSMAQKRPACTLKPECVQQLLVCSQEAKKSAYCPYSHFPVGAA |
| | LLTQEGRIFKGCNIENACYPLGICAERTAIQKAVSEGYKDFRAIAIASDMQDDFISPC |
| | GACRQVMREFGTNWPVYMTKPDGTYIVMTVQELLPSSFGPEDLQKTQHHHHHH |
| | (SEQ ID NO: 26) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)6 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSMAQKRPACTLKPECVQQLLVCSQEAKKSAYCPYSHF |
| | PVGAALLTQEGRIFKGCNIENACYPLGICAERTAIQKAVSEGYKDFRAIAIASDMQD |
| | DFISPCGACRQVMREFGTNWPVYMTKPDGTYIVMTVQELLPSSFGPEDLQKTQHH |
| | HHHH (SEQ ID NO: 27) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)7 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSGGGGSMAQKRPACTLKPECVQQLLVCSQEAKKSAYC |
| | PYSHFPVGAALLTQEGRIFKGCNIENACYPLGICAERTAIQKAVSEGYKDFRAIAIAS |
| | DMQDDFISPCGACRQVMREFGTNWPVYMTKPDGTYIVMTVQELLPSSFGPEDLQK |
| | TQHHHHHH (SEQ ID NO: 28) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)8 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSGGGGGGGGSMAQKRPACTLKPECVQQLLVCSQEAK |
| | KSAYCPYSHFPVGAALLTQEGRIFKGCNIENACYPLGICAERTAIQKAVSEGYKDFR |
| | AIAIASDMQDDFISPCGACRQVMREFGTNWPVYMTKPDGTYIVMTVQELLPSSFGP |
| | EDLQKTQHHHHHH (SEQ ID NO: 29) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)9 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSMAQKRPACTLKPECVQQLLV |
| | CSQEAKKSAYCPYSHFPVGAALLTQEGRIFKGCNIENACYPLGICAERTAIQKAVSE |
| | GYKDFRAIAIASDMQDDFISPCGACRQVMREFGTNWPVYMTKPDGTYIVMTVQEL |
| | LPSSFGPEDLQKTQHHHHHH (SEQ ID NO: 30) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)10 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQHHHHHH (SEQ ID NO: 31) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)11 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQHHHHHH (SEQ ID NO: 32) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)112 | GGSGGGGSEVQLVESGGGLVQPGGSLRLSCAASGETFSDHYMDWVRQAPGKGLA |
| linker_CDA_ | SGETFSDHYMDWVRQAPGKGLEWVGRIRNKASSYTTEYAASVKGRFTISRDDSKN |
| HIS | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSGGGGSGGGGSGGGGSMAQKR |
| | PACTLKPECVQQLLVCSQEAKKSAYCPYSHFPVGAALLTQEGRIFKGCNIENACYP |
| | LGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 33) |
| Ab68_LC_ | DIQMTQSPSSLSASVGDRVTITCRASQSISSYLNWYQQKPGKAPKLLIYAASSLQSG |
| (G4S)3_HC_G4S | VPSRFSGSGSGTDFTLTISSLQPEDFATYYCQQSYIAPYTFGGGTKVEIKGGGGSGG |
| linker_CDA_ | GGSGGGGSEVQLVESGGGLVQPGRSLRLSCTASGFTFSDHDMNWVRQAPGKGLE |
| HIS | WVGRTRNAAGSYTTEYAASVKGRFTISRDDSKNSLYLQMNSLKTEDTAVYYCAR |
| | EPKYWIDFDLWGRGTLVTSSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQHHHHHH (SEQ ID NO: 34) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)3 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQHHHHHH (SEQ ID NO: 35) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)4 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQHHHHHH (SEQ ID NO: 36) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)5 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQHHHHHH (SEQ ID NO: 37) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)6 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSMAQ |
| | KRPACTLKPECVQQLLVCSQEAKKSAYCPYSHFPVGAALLTQEGRIFKGCNIENAC |
| | YPLGICAERTAIQKAVSEGYKDFRAIAIASDMQDDFISPCGACRQVMREFGTNWPV |
| | YMTKPDGTYIVMTVQELLPSSFGPEDLQKTQHHHHHH (SEQ ID NO: 38) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)7 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQHHHHHH (SEQ ID NO: 39) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)8 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQHHHHHH (SEQ ID |
| | NO: 40) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)9 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSGGGG |
| | SGGGGGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 41) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)10 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQHHHH |
| | HH (SEQ ID NO: 42) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)11 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | QHHHHHH (SEQ ID NO: 43) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)112 | GGSGGGGSEVQLVESGGGLVQPGRSLRLSCTASGFTFSDHDMNWVRQAPGKGLE |
| linker_CDA_ | WVGRTRNAAGSYTTEYAASVKGRFTISRDDSKNSLYLQMNSLKTEDTAVYYCAR |
| HIS | EPKYWIDFDLWGRGTLVTSSGGGGSGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQHHHHHH (SEQ ID NO: 44) |
| Ab69_LC_ | EVQLVESGGGLVQPGGSLRLSCAASGETFVDHDMDWVRQAPGKGLEWVGRTRN |
| (G4S)3_HC_G4S | KLGSYTTEYAASVKGRETGRDDSKNSLYLQMNSLKTEDTAVYYCAREPKYWIDF |
| linker_CDA_ | DLWGRGTLVTVSSGGGGSGGGGSGGGGSDIQMTQSPSSLSASVGDRVTITCRASQS |
| HIS | ISSYLNWYQQKPGKAPKLLIYAASSLQSGVPSRFSGSGSGTDFTLTISSLQPEDFATY |
| | YCQQSYIAPYIFGGGTKVEIKGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQHHHHHH (SEQ ID NO: 45) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)3 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQHHHHHH (SEQ ID NO: 46) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)4 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQHHHHHH (SEQ ID NO: 47) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)5 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQHHHHHH (SEQ ID NO: 48) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)6 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSGGGGSGGGGSGGGGSMAQ |
| | KRPACTLKPECVQQLLVCSQEAKKSAYCPYSHFPVGAALLTQEGRIFKGCNIENAC |
| | YPLGICAERTAIQKAVSEGYKDFRAIAIASDMQDDFISPCGACRQVMREFGTNWPV |
| | YMTKPDGTYIVMTVQELLPSSFGPEDLQKTQHHHHHH (SEQ ID NO: 49) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)7 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQHHHHHH (SEQ ID NO: 50) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)8 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQHHHHHH (SEQ ID |
| | NO: 51) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)9 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 52) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)10 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQHHHH |
| | HH (SEQ ID NO: 53) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)11 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | QHHHHHH (SEQ ID NO: 54) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)112 | DLWGRGTLVTVSSGGGGSGGGGSGGGGSDIQMTQSPSSLSASVGDRVTITCRASQS |
| linker_CDA_ | ISSYLNWYQQKPGKAPKLLIYAASSLQSGVPSRFSGSGSGTDFTLTISSLQPEDFATY |
| HIS | YCQQSYIAPYIFGGGTKVEIKGGGGSGGGGSGGGGSGGGGSGGGGSGGGGSGGGG |
| | SGGGGSGGGGGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQHHHHHH (SEQ ID NO: 55) |
| Ab58_HC_ | EVQLLESGGGLVQPGGSLRLSCAASGETFSNYAMSWVRQAPGKGLEWVSAISGSG |
| (G4S)3_LC_G4S | GSTYYADSVKGRFTISRDNSKNTLYLQMNSLRAEDTAVYYCAKGPPTYHTNYYY |
| linker_CDA_ | MDVWGKGTTVTVSSGGGGSGGGGSGGGGSDIQMTQSPSSVSASVGDRVTITCRAS |
| HIS | QGISSWLAWYQQKPGKAPKLLLIYAASSLQSGVPSRFSGSGSGTDFTLTISSLQPED |
| | FATYYCQQTNSFPYTFGGGTKVEIKGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQHHHHHH (SEQ ID NO: 56) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)3 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQHHHHHH (SEQ ID NO: 57) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)4 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQHHHHHH (SEQ ID NO: 58) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)5 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 59) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)6 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQHHHHHH (SEQ ID NO: 60) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)7 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQHHHHHH (SEQ ID |
| | NO: 61) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)8 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 62) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)9 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQHHHH |
| | HH (SEQ ID NO: 63) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)10 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | QHHHHHH (SEQ ID NO: 64) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)11 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQHHHHHH (SEQ ID NO: 65) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)112 | MDVWGKGTTVTVSSGGGGSGGGGSGGGGSDIQMTQSPSSVSASVGDRVTITCRAS |
| linker_CDA_ | QGISSWLAWYQQKPGKAPKLLLIYAASSLQSGVPSRFSGSGSGTDFTLTISSLQPED |
| HIS | FATYYCQQTNSFPYTFGGGTKVEIKGGGGSGGGGSGGGGSGGGGSGGGGSGGGG |
| | SGGGGSGGGGSGGGGGGGGSGGGGSGGGGSMAQKRPACTLKPECVQQLLVCSQ |
| | EAKKSAYCPYSHFPVGAALLTQEGRIFKGCNIENACYPLGICAERTAIQKAVSEGY |
| | KDFRAIAIASDMQDDFISPCGACRQVMREFGTNWPVYMTKPDGTYIVMTVQELLP |
| | SSFGPEDLQKTQHHHHHH (SEQ ID NO: 66) |
| Ab61_HC_ | EVQLLESGGGLVQPGGSLRLSCAASGETESSYVMIWVRQAPGKGLEWVSSISGDSV |
| (G4S)3_LC_G4S | TTYYADSVKGRFTISRDNSKNTLYLQMNSLRAEDTAVYYCAKGPPTYHTNYYYM |
| linker_CDA_ | DVWGKGTTVTVSSGGGGSGGGGSGGGGSDIQMTQSPSSVSASVGDRVTITCRASQ |
| HIS | GISSWLAWYQQKPGKAPKLLIYAASSLQQSGVPSRFSGSGSGTDFTLTISSLQPEDF |
| | ATYYCQQTNSEPYTFGGGTKVEIKGGGGSMAQKRPACTLKPECVQQLLVCSQEAK |
| | KSAYCPYSHFPVGAALLTQEGRIFKGCNIENACYPLGICAERTAIQKAVSEGYKDFR |
| | AIAIASDMQDDFISPCGACRQVMREFGTNWPVYMTKPDGTYIVMTVQELLPSSFGP |
| | EDLQKTQHHHHHH (SEQ ID NO: 67) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)3 | DVWGKGTTVTVSSGGGGGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQHHHHHH (SEQ ID NO: 68) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)4 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQHHHHHH (SEQ ID NO: 69) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)5 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSMAQKR |
| | PACTLKPECVQQLLVCSQEAKKSAYCPYSHFPVGAALLTQEGRIFKGCNIENACYP |
| | LGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 70) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)6 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | MAQKRPACTLKPECVQQLLVCSQEAKKSAYCPYSHFPVGAALLTQEGRIFKGCNIE |
| | NACYPLGICAERTAIQKAVSEGYKDFRAIAIASDMQDDFISPCGACRQVMREFGTN |
| | WPVYMTKPDGTYIVMTVQELLPSSFGPEDLQKTQHHHHHH (SEQ ID NO: 71) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)7 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | GGGGSMAQKRPACTLKPECVQQLLVCSQEAKKSAYCPYSHFPVGAALLTQEGRIF |
| | KGCNIENACYPLGICAERTAIQKAVSEGYKDFRAIAIASDMQDDFISPCGACRQVM |
| | REFGTNWPVYMTKPDGTYIVMTVQELLPSSFGPEDLQKTQHHHHHH (SEQ ID NO: |
| | 72) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)8 | DVWGKGTTVTVSSGGGGGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | GGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 73) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)9 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | GGGGSGGGGSGGGGSMAQKRPACTLKPECVQQLLVCSQEAKKSAYCPYSHFPVG |
| | AALLTQEGRIFKGCNIENACYPLGICAERTAIQKAVSEGYKDFRAIAIASDMQDDFI |
| | SPCGACRQVMREFGTNWPVYMTKPDGTYIVMTVQELLPSSFGPEDLQKTQHHHH |
| | HH (SEQ ID NO: 74) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)10 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | GGGGSGGGGGGGGSGGGGSMAQKRPACTLKPECVQQLLVCSQEAKKSAYCPYS |
| | HFPVGAALLTQEGRIFKGCNIENACYPLGICAERTAIQKAVSEGYKDFRAIAIASDM |
| | QDDFISPCGACRQVMREFGTNWPVYMTKPDGTYIVMTVQELLPSSFGPEDLQKTQ |
| | HHHHHH (SEQ ID NO: 75) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)11 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | GGGGSGGGGGGGGSGGGGSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQHHHHHH (SEQ ID NO: 76) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)112 | DVWGKGTTVTVSSGGGGSGGGGSGGGGSDIQMTQSPSSVSASVGDRVTITCRASQ |
| linker_CDA_ | GISSWLAWYQQKPGKAPKLLIYAASSLQQSGVPSRFSGSGSGTDFTLTISSLQPEDF |
| HIS | ATYYCQQTNSEPYTFGGGTKVEIKGGGGSGGGGSGGGGSGGGGSGGGGSGGGGS |
| | GGGGSGGGGSGGGGSGGGGSGGGGSGGGGSMAQKRPACTLKPECVQQLLVCSQE |
| | AKKSAYCPYSHFPVGAALLTQEGRIFKGCNIENACYPLGICAERTAIQKAVSEGYK |
| | DFRAIAIASDMQDDFISPCGACRQVMREFGTNWPVYMTKPDGTYIVMTVQELLPSS |
| | FGPEDLQKTQHHHHHH (SEQ ID NO: 77) |
| Ab66_HC_ | EVQLVESGGGLVQPGGSLRLSCAASGETFSDHYMDWVRQAPGKGLASGETFSDH |
| (G4S)3_LC_G4S | YMDWVRQAPGKGLEWVGRIRNKASSYTTEYAASVKGRFTISRDDSKNSLYLQMN |
| linker_CDA_ | SLKTEDTAVYYCAREPKYWIDFDLWGRGTLVTVSSGGGGSGGGGSGGGGSDIQM |
| HIS | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSMAQKRP |
| | ACTLKPECVQQLLVCSQEAKKSAYCPYSHFPVGAALLTQEGRIFKGCNIENACYPL |
| | GICAERTAIQKAVSEGYKDFRAIAIASDMQDDFISPCGACRQVMREFGTNWPVYM |
| | TKPDGTYIVMTVQELLPSSFGPEDLQKTQHHHHHH (SEQ ID NO: 78) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)3 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSMAQKRPACTLKPECVQQLLVCSQEAKKSAYCPYSHFPVGAALLTQEGRIFK |
| | GCNIENACYPLGICAERTAIQKAVSEGYKDFRAIAIASDMQDDFISPCGACRQVMR |
| | EFGTNWPVYMTKPDGTYIVMTVQELLPSSFGPEDLQKTQHHHHHH (SEQ ID NO: |
| | 79) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)4 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSMAQKRPACTLKPECVQQLLVCSQEAKKSAYCPYSHFPVGAALLTQ |
| | EGRIFKGCNIENACYPLGICAERTAIQKAVSEGYKDFRAIAIASDMQDDFISPCGAC |
| | RQVMREFGTNWPVYMTKPDGTYIVMTVQELLPSSFGPEDLQKTQHHHHHH (SEQ |
| | ID NO: 80) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)5 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSMAQKRPACTLKPECVQQLLVCSQEAKKSAYCPYSHFPVGA |
| | ALLTQEGRIFKGCNIENACYPLGICAERTAIQKAVSEGYKDFRAIAIASDMQDDFISP |
| | CGACRQVMREFGTNWPVYMTKPDGTYIVMTVQELLPSSFGPEDLQKTQHHHHHH |
| | (SEQ ID NO: 81) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)6 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGGGGGSG |
| | GGGSGGGGGGGGSGGGGSMAQKRPACTLKPECVQQLLVCSQEAKKSAYCPYSH |
| | FPVGAALLTQEGRIFKGCNIENACYPLGICAERTAIQKAVSEGYKDFRAIAIASDMQ |
| | DDFISPCGACRQVMREFGTNWPVYMTKPDGTYIVMTVQELLPSSFGPEDLQKTQH |
| | HHHHH (SEQ ID NO: 82) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)7 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSGGGGSGGGGSMAQKRPACTLKPECVQQLLVCSQEAKKSAY |
| | CPYSHFPVGAALLTQEGRIFKGCNIENACYPLGICAERTAIQKAVSEGYKDFRAIAI |
| | ASDMQDDFISPCGACRQVMREFGTNWPVYMTKPDGTYIVMTVQELLPSSFGPEDL |
| | QKTQHHHHHH (SEQ ID NO: 83) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)8 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSGGGGSGGGGSGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQHHHHHH (SEQ ID NO: 84) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)9 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQHHHHHH (SEQ ID NO: 85) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)10 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQHHHHHH (SEQ ID NO: 86) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)11 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQHHHHHH (SEQ ID NO: 87) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)112 | SLKTEDTAVYYCAREPKYWIDFDLWGRGTLVTVSSGGGGSGGGGSGGGGSDIQM |
| linker_CDA_ | TQSPSSLSASVGDRVTITCRASQSISSYLNWYQQKPGKAPKLLIYAASSLQSGVPSR |
| HIS | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 88) |
| Ab68_HC_ | EVQLVESGGGLVQPGRSLRLSCTASGFTFSDHDMNWVRQAPGKGLEWVGRTRNA |
| (G4S)3_LC_G4S | AGSYTTEYAASVKGRFTISRDDSKNSLYLQMNSLKTEDTAVYYCAREPKYWIDFD |
| linker_CDA_ | LWGRGTLVTSSGGGGSGGGGSGGGGSDIQMTQSPSSLSASVGDRVTITCRASQSISS |
| HIS | YLNWYQQKPGKAPKLLIYAASSLQSGVPSRFSGSGSGTDFTLTISSLQPEDFATYYC |
| | QQSYIAPYTFGGGTKVEIKGGGGSMAQKRPACTLKPECVQQLLVCSQEAKKSAYC |
| | PYSHFPVGAALLTQEGRIFKGCNIENACYPLGICAERTAIQKAVSEGYKDFRAIAIAS |
| | DMQDDFISPCGACRQVMREFGTNWPVYMTKPDGTYIVMTVQELLPSSFGPEDLQK |
| | TQHHHHHH (SEQ ID NO: 89) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)3 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSMAQKRPACTLKPECVQQLLVC |
| | SQEAKKSAYCPYSHFPVGAALLTQEGRIFKGCNIENACYPLGICAERTAIQKAVSEG |
| | YKDFRAIAIASDMQDDFISPCGACRQVMREFGTNWPVYMTKPDGTYIVMTVQELL |
| | PSSFGPEDLQKTQHHHHHH (SEQ ID NO: 90) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)4 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSMAQKRPACTLKPECVQ |
| | QLLVCSQEAKKSAYCPYSHFPVGAALLTQEGRIFKGCNIENACYPLGICAERTAIQK |
| | AVSEGYKDFRAIAIASDMQDDFISPCGACRQVMREFGTNWPVYMTKPDGTYIVMT |
| | VQELLPSSFGPEDLQKTQHHHHHH (SEQ ID NO: 91) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)5 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQHHHHHH (SEQ ID NO: 92) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)6 | LWGRGTLVTSSGGGGGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGGGGGSGGGGSGGGGSGGGGSGGGGSMAQKR |
| | PACTLKPECVQQLLVCSQEAKKSAYCPYSHFPVGAALLTQEGRIFKGCNIENACYP |
| | LGICAERTAIQKAVSEGYKDFRAIAIASDMQDDFISPCGACRQVMREFGTNWPVY |
| | MTKPDGTYIVMTVQELLPSSFGPEDLQKTQHHHHHH (SEQ ID NO: 93) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)7 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSGGGGSGGGGS |
| | MAQKRPACTLKPECVQQLLVCSQEAKKSAYCPYSHFPVGAALLTQEGRIFKGCNIE |
| | NACYPLGICAERTAIQKAVSEGYKDFRAIAIASDMQDDFISPCGACRQVMREFGTN |
| | WPVYMTKPDGTYIVMTVQELLPSSFGPEDLQKTQHHHHHH (SEQ ID NO: 94) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)8 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSGGGGSGGGGS |
| | GGGGSMAQKRPACTLKPECVQQLLVCSQEAKKSAYCPYSHFPVGAALLTQEGRIF |
| | KGCNIENACYPLGICAERTAIQKAVSEGYKDFRAIAIASDMQDDFISPCGACRQVM |
| | REFGTNWPVYMTKPDGTYIVMTVQELLPSSFGPEDLQKTQHHHHHH (SEQ ID NO: |
| | 95) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)9 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSGGGGSGGGGS |
| | GGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQHHHHHH |
| | (SEQ ID NO: 96) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)10 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSGGGGSGGGGS |
| | GGGGSGGGGSGGGGSMAQKRPACTLKPECVQQLLVCSQEAKKSAYCPYSHFPVG |
| | AALLTQEGRIFKGCNIENACYPLGICAERTAIQKAVSEGYKDFRAIAIASDMQDDFI |
| | SPCGACRQVMREFGTNWPVYMTKPDGTYIVMTVQELLPSSFGPEDLQKTQHHHH |
| | HH (SEQ ID NO: 97) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)11 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSGGGGSGGGGS |
| | GGGGSGGGGSGGGGSGGGGSMAQKRPACTLKPECVQQLLVCSQEAKKSAYCPYS |
| | HFPVGAALLTQEGRIFKGCNIENACYPLGICAERTAIQKAVSEGYKDFRAIAIASDM |
| | QDDFISPCGACRQVMREFGTNWPVYMTKPDGTYIVMTVQELLPSSFGPEDLQKTQ |
| | HHHHHH (SEQ ID NO: 98) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)112 | LWGRGTLVTSSGGGGSGGGGSGGGGSDIQMTQSPSSLSASVGDRVTITCRASQSISS |
| linker_CDA_ | YLNWYQQKPGKAPKLLIYAASSLQSGVPSRFSGSGSGTDFTLTISSLQPEDFATYYC |
| HIS | QQSYIAPYTFGGGTKVEIKGGGGSGGGGSGGGGSGGGGSGGGGSGGGGGGGGS |
| | GGGGSGGGGSGGGGSGGGGSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQHHHHHH (SEQ ID NO: 99) |
| Ab69_HC_ | DIQMTQSPSSLSASVGDRVTITCRASQSISSYLNWYQQKPGKAPKLLIYAASSLQSG |
| (G4S)3_LC_G4S | VPSRFSGSGSGTDFTLTISSLQPEDFATYYCQQSYIAPYIFGGGTKVEIKGGGGSGGG |
| linker_CDA_ | GSGGGGSEVQLVESGGGLVQPGGSLRLSCAASGETFVDHDMDWVRQAPGKGLE |
| HIS | WVGRTRNKLGSYTTEYAASVKGRETGRDDSKNSLYLQMNSLKTEDTAVYYCARE |
| | PKYWIDFDLWGRGTLVTVSSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQHHHHHH (SEQ ID NO: 100) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)3 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQHHHHHH (SEQ ID NO: 101) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)4 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQHHHHHH (SEQ ID NO: 102) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)5 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSMAQKRPAC |
| | TLKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQHHHHHH (SEQ ID NO: 103) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)6 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSMAQ |
| | KRPACTLKPECVQQLLVCSQEAKKSAYCPYSHFPVGAALLTQEGRIFKGCNIENAC |
| | YPLGICAERTAIQKAVSEGYKDFRAIAIASDMQDDFISPCGACRQVMREFGTNWPV |
| | YMTKPDGTYIVMTVQELLPSSFGPEDLQKTQHHHHHH (SEQ ID NO: 104) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)7 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSGGG |
| | GSMAQKRPACTLKPECVQQLLVCSQEAKKSAYCPYSHFPVGAALLTQEGRIFKGC |
| | NIENACYPLGICAERTAIQKAVSEGYKDFRAIAIASDMQDDFISPCGACRQVMREFG |
| | TNWPVYMTKPDGTYIVMTVQELLPSSFGPEDLQKTQHHHHHH (SEQ ID NO: 105) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)8 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSGGG |
| | GSGGGGSMAQKRPACTLKPECVQQLLVCSQEAKKSAYCPYSHFPVGAALLTQEGR |
| | IFKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQHHHHHH (SEQ ID |
| | NO: 106) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)9 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSGGG |
| | GSGGGGSGGGGSMAQKRPACTLKPECVQQLLVCSQEAKKSAYCPYSHFPVGAAL |
| | LTQEGRIFKGCNIENACYPLGICAERTAIQKAVSEGYKDFRAIAIASDMQDDFISPCG |
| | ACRQVMREFGTNWPVYMTKPDGTYIVMTVQELLPSSFGPEDLQKTQHHHHHH |
| | (SEQ ID NO: 107) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)10 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSGGG |
| | GSGGGGSGGGGSGGGGSMAQKRPACTLKPECVQQLLVCSQEAKKSAYCPYSHFP |
| | VGAALLTQEGRIFKGCNIENACYPLGICAERTAIQKAVSEGYKDFRAIAIASDMQD |
| | DFISPCGACRQVMREFGTNWPVYMTKPDGTYIVMTVQELLPSSFGPEDLQKTQHH |
| | HHHH (SEQ ID NO: 108) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)11 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSGGG |
| | GSGGGGSGGGGSGGGGSGGGGSMAQKRPACTLKPECVQQLLVCSQEAKKSAYCP |
| | YSHFPVGAALLTQEGRIFKGCNIENACYPLGICAERTAIQKAVSEGYKDFRAIAIAS |
| | DMQDDFISPCGACRQVMREFGTNWPVYMTKPDGTYIVMTVQELLPSSFGPEDLQK |
| | TQHHHHHH (SEQ ID NO: 109) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)112 | GSGGGGSEVQLVESGGGLVQPGGSLRLSCAASGETFVDHDMDWVRQAPGKGLE |
| linker_CDA_ | WVGRTRNKLGSYTTEYAASVKGRETGRDDSKNSLYLQMNSLKTEDTAVYYCARE |
| HIS | PKYWIDFDLWGRGTLVTVSSGGGGSGGGGSGGGGSGGGGSGGGGSGGGGSGGG |
| | GSGGGGSGGGGSGGGGSGGGGSGGGGSMAQKRPACTLKPECVQQLLVCSQEAKK |
| | SAYCPYSHFPVGAALLTQEGRIFKGCNIENACYPLGICAERTAIQKAVSEGYKDFRA |
| | IAIASDMQDDFISPCGACRQVMREFGTNWPVYMTKPDGTYIVMTVQELLPSSFGPE |
| | DLQKTQHHHHHH (SEQ ID NO: 110) |
| CDA_G4S | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSVSASVG |
| HC_HIS | DRVTITCRASQGISSWLAWYQQKPGKAPKLLLIYAASSLQSGVPSRFSGSGSGTDFT |
| | LTISSLQPEDFATYYCQQTNSFPYTFGGGTKVEIKGGGGSGGGGSGGGGSEVQLLE |
| | SGGGLVQPGGSLRLSCAASGETFSNYAMSWVRQAPGKGLEWVSAISGSGGSTYYA |
| | DSVKGRFTISRDNSKNTLYLQMNSLRAEDTAVYYCAKGPPTYHTNYYYMDVWGK |
| | GTTVTVSSHHHHHH (SEQ ID NO: 111) |
| CDA_G4S | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSVSASVG |
| HC_HIS | DRVTITCRASQGISSWLAWYQQKPGKAPKLLIYAASSLQQSGVPSRFSGSGSGTDF |
| | TLTISSLQPEDFATYYCQQTNSEPYTFGGGTKVEIKGGGGSGGGGSGGGGSEVQLL |
| | ESGGGLVQPGGSLRLSCAASGETESSYVMIWVRQAPGKGLEWVSSISGDSVTTYY |
| | ADSVKGRFTISRDNSKNTLYLQMNSLRAEDTAVYYCAKGPPTYHTNYYYMDVWG |
| | KGTTVTVSSHHHHHH (SEQ ID NO: 112) |
| CDA_G4S | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSLSASVG |
| HC_HIS | DRVTITCRASQSISSYLNWYQQKPGKAPKLLIYAASSLQSGVPSRFSGSGSGTDFTL |
| | TISSLQPEDFATYYCQQSYIAPYTFGGGTKVEIKGGGGSGGGGSGGGGSEVQLVES |
| | GGGLVQPGGSLRLSCAASGETFSDHYMDWVRQAPGKGLASGETFSDHYMDWVR |
| | QAPGKGL |
| | EWVGRIRNKASSYTTEYAASVKGRFTISRDDSKNSLYLQMNSLKTEDTAVYYCAR |
| | EPKYWIDFDLWGRGTLVTVSSHHHHHH (SEQ ID NO: 113) |
| CDA_G4S | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSLSASVG |
| HC_HIS | DRVTITCRASQSISSYLNWYQQKPGKAPKLLIYAASSLQSGVPSRFSGSGSGTDFTL |
| | TISSLQPEDFATYYCQQSYIAPYTFGGGTKVEIKGGGGSGGGGSGGGGSEVQLVES |
| | GGGLVQPGRSLRLSCTASGFTFSDHDMNWVRQAPGKGLEWVGRTRNAAGSYTTE |
| | YAASVKGRFTISRDDSKNSLYLQMNSLKTEDTAVYYCAREPKYWIDFDLWGRGTL |
| | VTSSHHHHHH (SEQ ID NO: 114) |
| CDA_G4S | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSEVQLVESGGGLVQPGG |
| HC_HIS | SLRLSCAASGETFVDHDMDWVRQAPGKGLEWVGRTRNKLGSYTTEYAASVKGRE |
| | TGRDDSKNSLYLQMNSLKTEDTAVYYCAREPKYWIDFDLWGRGTLVTVSSGGGG |
| | SGGGGSGGGGSDIQMTQSPSSLSASVGDRVTITCRASQSISSYLNWYQQKPGKAPK |
| | LLIYAASSLQSGVPSRFSGSGSGTDFTLTISSLQPEDFATYYCQQSYIAPYIFGGGTK |
| | VEIKHHHHHH (SEQ ID NO: 115) |
| CDA_(G4S)3 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC_HIS | QSPSSVSASVGDRVTITCRASQGISSWLAWYQQKPGKAPKLLLIYAASSLQSGVPSR |
| | FSGSGSGTDFTLTISSLQPEDFATYYCQQTNSFPYTFGGGTKVEIKGGGGSGGGGSG |
| | GGGSEVQLLESGGGLVQPGGSLRLSCAASGETFSNYAMSWVRQAPGKGLEWVSAI |
| | SGSGGSTYYADSVKGRFTISRDNSKNTLYLQMNSLRAEDTAVYYCAKGPPTYHTN |
| | YYYMDVWGKGTTVTVSSHHHHHH (SEQ ID NO: 116) |
| CDA_(G4S)3 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC_HIS | QSPSSVSASVGDRVTITCRASQGISSWLAWYQQKPGKAPKLLIYAASSLQQSGVPS |
| | RFSGSGSGTDFTLTISSLQPEDFATYYCQQTNSEPYTFGGGTKVEIKGGGGSGGGGS |
| | GGGGSEVQLLESGGGLVQPGGSLRLSCAASGETESSYVMIWVRQAPGKGLEWVSS |
| | ISGDSVTTYYADSVKGRFTISRDNSKNTLYLQMNSLRAEDTAVYYCAKGPPTYHT |
| | NYYYMDVWGKGTTVTVSSHHHHHH (SEQ ID NO: 117) |
| CDA_(G4S)3 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC_HIS | QSPSSLSASVGDRVTITCRASQSISSYLNWYQQKPGKAPKLLIYAASSLQSGVPSRFS |
| | GSGSGTDFTLTISSLQPEDFATYYCQQSYIAPYTFGGGTKVEIKGGGGSGGGGSGG |
| | GGSEVQLVESGGGLVQPGGSLRLSCAASGETFSDHYMDWVRQAPGKGLASGETFS |
| | DHYMDWVRQAPGKGLEWVGRIRNKASSYTTEYAASVKGRFTISRDDSKNSLYLQ |
| | MNSLKTEDTAVYYCAREPKYWIDFDLWGRGTLVTVSSHHHHHH (SEQ ID NO: |
| | 118) |
| CDA_(G4S)3 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC_HIS | QSPSSLSASVGDRVTITCRASQSISSYLNWYQQKPGKAPKLLIYAASSLQSGVPSRFS |
| | GSGSGTDFTLTISSLQPEDFATYYCQQSYIAPYTFGGGTKVEIKGGGGSGGGGSGG |
| | GGSEVQLVESGGGLVQPGRSLRLSCTASGFTFSDHDMNWVRQAPGKGLEWVGRT |
| | RNAAGSYTTEYAASVKGRFTISRDDSKNSLYLQMNSLKTEDTAVYYCAREPKYWI |
| | DFDLWGRGTLVTSSHHHHHH (SEQ ID NO: 119) |
| CDA_(G4S)3 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSEVQLV |
| HC_HIS | ESGGGLVQPGGSLRLSCAASGETFVDHDMDWVRQAPGKGLEWVGRTRNKLGSYT |
| | TEYAASVKGRETGRDDSKNSLYLQMNSLKTEDTAVYYCAREPKYWIDFDLWGRG |
| | TLVTVSSGGGGSGGGGSGGGGSDIQMTQSPSSLSASVGDRVTITCRASQSISSYLN |
| | WYQQKPGKAPKLLIYAASSLQSGVPSRFSGSGSGTDFTLTISSLQPEDFATYYCQQS |
| | YIAPYIFGGGTKVEIKHHHHHH (SEQ ID NO: 120) |
| CDA_(G4S)4 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | DIQMTQSPSSVSASVGDRVTITCRASQGISSWLAWYQQKPGKAPKLLLIYAASSLQ |
| | SGVPSRFSGSGSGTDFTLTISSLQPEDFATYYCQQTNSFPYTFGGGTKVEIKGGGGS |
| | GGGGSGGGGSEVQLLESGGGLVQPGGSLRLSCAASGETFSNYAMSWVRQAPGKG |
| | LEWVSAISGSGGSTYYADSVKGRFTISRDNSKNTLYLQMNSLRAEDTAVYYCAKG |
| | PPTYHTNYYYMDVWGKGTTVTVSSHHHHHH (SEQ ID NO: 121) |
| CDA_(G4S)4 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | DIQMTQSPSSVSASVGDRVTITCRASQGISSWLAWYQQKPGKAPKLLIYAASSLQQ |
| | SGVPSRFSGSGSGTDFTLTISSLQPEDFATYYCQQTNSEPYTFGGGTKVEIKGGGGS |
| | GGGGSGGGGSEVQLLESGGGLVQPGGSLRLSCAASGETESSYVMIWVRQAPGKGL |
| | EWVSSISGDSVTTYYADSVKGRFTISRDNSKNTLYLQMNSLRAEDTAVYYCAKGP |
| | PTYHTNYYYMDVWGKGTTVTVSSHHHHHH (SEQ ID NO: 122) |
| CDA_(G4S)4 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYTFGGGTKVEIKGGGGSGG |
| | GGSGGGGSEVQLVESGGGLVQPGGSLRLSCAASGETFSDHYMDWVRQAPGKGLA |
| | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSHHHHHH (SEQ ID |
| | NO: 123) |
| CDA_(G4S)4 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYTFGGGTKVEIKGGGGSGG |
| | GGSGGGGSEVQLVESGGGLVQPGRSLRLSCTASGFTFSDHDMNWVRQAPGKGLE |
| | WVGRTRNAAGSYTTEYAASVKGRFTISRDDSKNSLYLQMNSLKTEDTAVYYCAR |
| | EPKYWIDFDLWGRGTLVTSSHHHHHH (SEQ ID NO: 124) |
| CDA_(G4S)4 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | EVQLVESGGGLVQPGGSLRLSCAASGETFVDHDMDWVRQAPGKGLEWVGRTRN |
| | KLGSYTTEYAASVKGRETGRDDSKNSLYLQMNSLKTEDTAVYYCAREPKYWIDF |
| | DLWGRGTLVTVSSGGGGSGGGGSGGGGSDIQMTQSPSSLSASVGDRVTITCRASQS |
| | ISSYLNWYQQKPGKAPKLLIYAASSLQSGVPSRFSGSGSGTDFTLTISSLQPEDFATY |
| | YCQQSYIAPYIFGGGTKVEIKHHHHHH (SEQ ID NO: 125) |
| CDA_(G4S)5 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSDIQMTQSPSSVSASVGDRVTITCRASQGISSWLAWYQQKPGKAPKLLLIYA |
| | ASSLQSGVPSRFSGSGSGTDFTLTISSLQPEDFATYYCQQTNSFPYTFGGGTKVEIKG |
| | GGGSGGGGSGGGGSEVQLLESGGGLVQPGGSLRLSCAASGETFSNYAMSWVRQA |
| | PGKGLEWVSAISGSGGSTYYADSVKGRFTISRDNSKNTLYLQMNSLRAEDTAVYY |
| | CAKGPPTYHTNYYYMDVWGKGTTVTVSSHHHHHH (SEQ ID NO: 126) |
| CDA_(G4S)5 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGGGGGSGGGGSGGGGS |
| HC_HIS | GGGGSDIQMTQSPSSVSASVGDRVTITCRASQGISSWLAWYQQKPGKAPKLLIYAA |
| | SSLQQSGVPSRFSGSGSGTDFTLTISSLQPEDFATYYCQQTNSEPYTFGGGTKVEIKG |
| | GGGSGGGGSGGGGSEVQLLESGGGLVQPGGSLRLSCAASGETESSYVMIWVRQAP |
| | GKGLEWVSSISGDSVTTYYADSVKGRFTISRDNSKNTLYLQMNSLRAEDTAVYYC |
| | AKGPPTYHTNYYYMDVWGKGTTVTVSSHHHHHH (SEQ ID NO: 127) |
| CDA_(G4S)5 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYTFGGGTKVEIKGGG |
| | GSGGGGGGGGSEVQLVESGGGLVQPGGSLRLSCAASGETFSDHYMDWVRQAPG |
| | KGLASGETFSDHYMDWVRQAPGKGLEWVGRIRNKASSYTTEYAASVKGRFTISRD |
| | DSKNSLYLQMNSLKTEDTAVYYCAREPKYWIDFDLWGRGTLVTVSSHHHHHH |
| | (SEQ ID NO: 128) |
| CDA_(G4S)5 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYTFGGGTKVEIKGGG |
| | GSGGGGSGGGGSEVQLVESGGGLVQPGRSLRLSCTASGFTFSDHDMNWVRQAPG |
| | KGLEWVGRTRNAAGSYTTEYAASVKGRFTISRDDSKNSLYLQMNSLKTEDTAVY |
| | YCAREPKYWIDFDLWGRGTLVTSSHHHHHH (SEQ ID NO: 129) |
| CDA_(G4S)5 | MAQKRPACTLKPECVQQLLVCSQEAKKSAYCPYSHFPVGAALLTQEGRIFKGCNIE |
| linke_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSEVQLVESGGGLVQPGGSLRLSCAASGETFVDHDMDWVRQAPGKGLEWV |
| | GRTRNKLGSYTTEYAASVKGRETGRDDSKNSLYLQMNSLKTEDTAVYYCAREPK |
| | YWIDFDLWGRGTLVTVSSGGGGSGGGGSGGGGSDIQMTQSPSSLSASVGDRVTIT |
| | CRASQSISSYLNWYQQKPGKAPKLLIYAASSLQSGVPSRFSGSGSGTDFTLTISSLQP |
| | EDFATYYCQQSYIAPYIFGGGTKVEIKHHHHHH (SEQ ID NO: 130) |
| CDA_(G4S)6 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSDIQMTQSPSSVSASVGDRVTITCRASQGISSWLAWYQQKPGKAPK |
| | LLLIYAASSLQSGVPSRFSGSGSGTDFTLTISSLQPEDFATYYCQQTNSFPYTFGGGT |
| | KVEIKGGGGSGGGGSGGGGSEVQLLESGGGLVQPGGSLRLSCAASGETFSNYAMS |
| | WVRQAPGKGLEWVSAISGSGGSTYYADSVKGRFTISRDNSKNTLYLQMNSLRAED |
| | TAVYYCAKGPPTYHTNYYYMDVWGKGTTVTVSSHHHHHH (SEQ ID NO: 131) |
| CDA_(G4S)6 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGGGGGSDIQMTQSPSSVSASVGDRVTITCRASQGISSWLAWYQQKPGKAPK |
| | LLIYAASSLQQSGVPSRFSGSGSGTDFTLTISSLQPEDFATYYCQQTNSEPYTFGGGT |
| | KVEIKGGGGSGGGGSGGGGSEVQLLESGGGLVQPGGSLRLSCAASGETESSYVMI |
| | WVRQAPGKGLEWVSSISGDSVTTYYADSVKGRFTISRDNSKNTLYLQMNSLRAED |
| | TAVYYCAKGPPTYHTNYYYMDVWGKGTTVTVSSHHHHHH (SEQ ID NO: 132) |
| CDA_(G4S)6 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIKGGGGSGGGGSGGGGSEVQLVESGGGLVQPGGSLRLSCAASGETFSDHYMDW |
| | VRQAPGKGLASGETFSDHYMDWVRQAPGKGLEWVGRIRNKASSYTTEYAASVKG |
| | RFTISRDDSKNSLYLQMNSLKTEDTAVYYCAREPKYWIDFDLWGRGTLVTVSSHH |
| | HHHH (SEQ ID NO: 133) |
| CDA_(G4S)6 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIKGGGGSGGGGSGGGGSEVQLVESGGGLVQPGRSLRLSCTASGFTFSDHDMNWV |
| | RQAPGKGLEWVGRTRNAAGSYTTEYAASVKGRFTISRDDSKNSLYLQMNSLKTED |
| | TAVYYCAREPKYWIDFDLWGRGTLVTSSHHHHHH (SEQ ID NO: 134) |
| CDA_(G4S)6 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGGGGGSEVQLVESGGGLVQPGGSLRLSCAASGETFVDHDMDWVRQAPGK |
| | GLEWVGRTRNKLGSYTTEYAASVKGRETGRDDSKNSLYLQMNSLKTEDTAVYYC |
| | AREPKYWIDFDLWGRGTLVTVSSGGGGSGGGGSGGGGSDIQMTQSPSSLSASVGD |
| | RVTITCRASQSISSYLNWYQQKPGKAPKLLIYAASSLQSGVPSRFSGSGSGTDFTLTI |
| | SSLQPEDFATYYCQQSYIAPYIFGGGTKVEIKHHHHHH (SEQ ID NO: 135) |
| CDA_(G4S)7 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSDIQMTQSPSSVSASVGDRVTITCRASQGISSWLAWYQQKP |
| | GKAPKLLLIYAASSLQSGVPSRFSGSGSGTDFTLTISSLQPEDFATYYCQQTNSFPYT |
| | FGGGTKVEIKGGGGSGGGGSGGGGSEVQLLESGGGLVQPGGSLRLSCAASGETFS |
| | NYAMSWVRQAPGKGLEWVSAISGSGGSTYYADSVKGRFTISRDNSKNTLYLQMN |
| | SLRAEDTAVYYCAKGPPTYHTNYYYMDVWGKGTTVTVSSHHHHHH (SEQ ID NO: |
| | 136) |
| CDA_(G4S)7 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSDIQMTQSPSSVSASVGDRVTITCRASQGISSWLAWYQQKP |
| | GKAPKLLIYAASSLQQSGVPSRFSGSGSGTDFTLTISSLQPEDFATYYCQQTNSEPYT |
| | FGGGTKVEIKGGGGSGGGGSGGGGSEVQLLESGGGLVQPGGSLRLSCAASGETESS |
| | YVMIWVRQAPGKGLEWVSSISGDSVTTYYADSVKGRFTISRDNSKNTLYLQMNSL |
| | RAEDTAVYYCAKGPPTYHTNYYYMDVWGKGTTVTVSSHHHHHH (SEQ ID NO: |
| | 137) |
| CDA_(G4S)7 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSDIQMTQSPSSLSASVGDRVTITCRASQSISSYLNWYQQKPG |
| | KAPKLLIYAASSLQSGVPSRFSGSGSGTDFTLTISSLQPEDFATYYCQQSYIAPYTFG |
| | GGTKVEIKGGGGSGGGGSGGGGSEVQLVESGGGLVQPGGSLRLSCAASGETFSDH |
| | YMDWVRQAPGKGLASGETFSDHYMDWVRQAPGKGLEWVGRIRNKASSYTTEYA |
| | ASVKGRFTISRDDSKNSLYLQMNSLKTEDTAVYYCAREPKYWIDFDLWGRGTLVT |
| | VSSHHHHHH (SEQ ID NO: 138) |
| CDA_(G4S)7 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSDIQMTQSPSSLSASVGDRVTITCRASQSISSYLNWYQQKPG |
| | KAPKLLIYAASSLQSGVPSRFSGSGSGTDFTLTISSLQPEDFATYYCQQSYIAPYTFG |
| | GGTKVEIKGGGGSGGGGSGGGGSEVQLVESGGGLVQPGRSLRLSCTASGFTFSDH |
| | DMNWVRQAPGKGLEWVGRTRNAAGSYTTEYAASVKGRFTISRDDSKNSLYLQM |
| | NSLKTEDTAVYYCAREPKYWIDFDLWGRGTLVTSSHHHHHH (SEQ ID NO: 139) |
| CDA_(G4S)7 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGGGGGSGGGGS |
| HC_HIS | GGGGSGGGGSGGGGSEVQLVESGGGLVQPGGSLRLSCAASGETFVDHDMDWVR |
| | QAPGKGLEWVGRTRNKLGSYTTEYAASVKGRETGRDDSKNSLYLQMNSLKTEDT |
| | AVYYCAREPKYWIDFDLWGRGTLVTVSSGGGGSGGGGSGGGGSDIQMTQSPSSLS |
| | ASVGDRVTITCRASQSISSYLNWYQQKPGKAPKLLIYAASSLQSGVPSRFSGSGSGT |
| | DFTLTISSLQPEDFATYYCQQSYIAPYIFGGGTKVEIKHHHHHH (SEQ ID NO: 140) |
| CDA_(G4S)8 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSDIQMTQSPSSVSASVGDRVTITCRASQGISSWLAW |
| | YQQKPGKAPKLLLIYAASSLQSGVPSRFSGSGSGTDFTLTISSLQPEDFATYYCQQT |
| | NSFPYTFGGGTKVEIKGGGGSGGGGSGGGGSEVQLLESGGGLVQPGGSLRLSCAA |
| | SGETFSNYAMSWVRQAPGKGLEWVSAISGSGGSTYYADSVKGRFTISRDNSKNTL |
| | YLQMNSLRAEDTAVYYCAKGPPTYHTNYYYMDVWGKGTTVTVSSHHHHHH |
| | (SEQ ID NO: 141) |
| CDA_(G4S)8 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSDIQMTQSPSSVSASVGDRVTITCRASQGISSWLAW |
| | YQQKPGKAPKLLIYAASSLQQSGVPSRFSGSGSGTDFTLTISSLQPEDFATYYCQQT |
| | NSEPYTFGGGTKVEIKGGGGSGGGGSGGGGSEVQLLESGGGLVQPGGSLRLSCAA |
| | SGETESSYVMIWVRQAPGKGLEWVSSISGDSVTTYYADSVKGRFTISRDNSKNTLY |
| | LQMNSLRAEDTAVYYCAKGPPTYHTNYYYMDVWGKGTTVTVSSHHHHHH (SEQ |
| | ID NO: 142) |
| CDA_(G4S)8 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYTFGGGTKVEIKGGGGSGGGGSGGGGSEVQLVESGGGLVQPGGSLRLSCAASGE |
| | TFSDHYMDWVRQAPGKGLASGETFSDHYMDWVRQAPGKGLEWVGRIRNKASSY |
| | TTEYAASVKGRFTISRDDSKNSLYLQMNSLKTEDTAVYYCAREPKYWIDFDLWGR |
| | GTLVTVSSHHHHHH (SEQ ID NO: 143) |
| CDA_(G4S)8 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYTFGGGTKVEIKGGGGSGGGGSGGGGSEVQLVESGGGLVQPGRSLRLSCTASGF |
| | TFSDHDMNWVRQAPGKGLEWVGRTRNAAGSYTTEYAASVKGRFTISRDDSKNSL |
| | YLQMNSLKTEDTAVYYCAREPKYWIDFDLWGRGTLVTSSHHHHHH (SEQ ID NO: |
| | 144) |
| CDA_(G4S)8 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSEVQLVESGGGLVQPGGSLRLSCAASGETFVDHDM |
| | DWVRQAPGKGLEWVGRTRNKLGSYTTEYAASVKGRETGRDDSKNSLYLQMNSL |
| | KTEDTAVYYCAREPKYWIDFDLWGRGTLVTVSSGGGGSGGGGSGGGGSDIQMTQ |
| | SPSSLSASVGDRVTITCRASQSISSYLNWYQQKPGKAPKLLIYAASSLQSGVPSRFSG |
| | SGSGTDFTLTISSLQPEDFATYYCQQSYIAPYIFGGGTKVEIKHHHHHH (SEQ ID NO: |
| | 145) |
| CDA_(G4S)9 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSDIQMTQSPSSVSASVGDRVTITCRASQGISS |
| | WLAWYQQKPGKAPKLLLIYAASSLQSGVPSRFSGSGSGTDFTLTISSLQPEDFATYY |
| | CQQTNSFPYTFGGGTKVEIKGGGGSGGGGSGGGGSEVQLLESGGGLVQPGGSLRL |
| | SCAASGETFSNYAMSWVRQAPGKGLEWVSAISGSGGSTYYADSVKGRFTISRDNS |
| | KNTLYLQMNSLRAEDTAVYYCAKGPPTYHTNYYYMDVWGKGTTVTVSSHHHHH |
| | H (SEQ ID NO: 146) |
| CDA_(G4S)9 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSDIQMTQSPSSVSASVGDRVTITCRASQGISS |
| | WLAWYQQKPGKAPKLLIYAASSLQQSGVPSRFSGSGSGTDFTLTISSLQPEDFATY |
| | YCQQTNSEPYTFGGGTKVEIKGGGGSGGGGSGGGGSEVQLLESGGGLVQPGGSLR |
| | LSCAASGETESSYVMIWVRQAPGKGLEWVSSISGDSVTTYYADSVKGRFTISRDNS |
| | KNTLYLQMNSLRAEDTAVYYCAKGPPTYHTNYYYMDVWGKGTTVTVSSHHHHH |
| | H (SEQ ID NO: 147) |
| CDA_(G4S)9 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGGGGGSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSEVQLVESGGGLVQPGGSLRLS |
| | CAASGETFSDHYMDWVRQAPGKGLASGETFSDHYMDWVRQAPGKGLEWVGRIR |
| | NKASSYTTEYAASVKGRFTISRDDSKNSLYLQMNSLKTEDTAVYYCAREPKYWID |
| | FDLWGRGTLVTVSSHHHHHH (SEQ ID NO: 148) |
| CDA_(G4S)9 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSEVQLVESGGGLVQPGRSLRLS |
| | CTASGFTFSDHDMNWVRQAPGKGLEWVGRTRNAAGSYTTEYAASVKGRFTISRD |
| | DSKNSLYLQMNSLKTEDTAVYYCAREPKYWIDFDLWGRGTLVTSSHHHHHH |
| | (SEQ ID NO: 149) |
| CDA_(G4S)9 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSEVQLVESGGGLVQPGGSLRLSCAASGETF |
| | VDHDMDWVRQAPGKGLEWVGRTRNKLGSYTTEYAASVKGRETGRDDSKNSLYL |
| | QMNSLKTEDTAVYYCAREPKYWIDFDLWGRGTLVTVSSGGGGSGGGGSGGGGSD |
| | IQMTQSPSSLSASVGDRVTITCRASQSISSYLNWYQQKPGKAPKLLIYAASSLQSGV |
| | PSRFSGSGSGTDFTLTISSLQPEDFATYYCQQSYIAPYIFGGGTKVEIKHHHHHH |
| | (SEQ ID NO: 150) |
| CDA_(G4S)10 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSDIQMTQSPSSVSASVGDRVTITCRA |
| | SQGISSWLAWYQQKPGKAPKLLLIYAASSLQSGVPSRFSGSGSGTDFTLTISSLQPE |
| | DFATYYCQQTNSFPYTFGGGTKVEIKGGGGSGGGGSGGGGSEVQLLESGGGLVQP |
| | GGSLRLSCAASGETFSNYAMSWVRQAPGKGLEWVSAISGSGGSTYYADSVKGRFT |
| | ISRDNSKNTLYLQMNSLRAEDTAVYYCAKGPPTYHTNYYYMDVWGKGTTVTVSS |
| | HHHHHH (SEQ ID NO: 151) |
| CDA_(G4S)10 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSDIQMTQSPSSVSASVGDRVTITCRA |
| | SQGISSWLAWYQQKPGKAPKLLIYAASSLQQSGVPSRFSGSGSGTDFTLTISSLQPE |
| | DFATYYCQQTNSEPYTFGGGTKVEIKGGGGSGGGGSGGGGSEVQLLESGGGLVQP |
| | GGSLRLSCAASGETESSYVMIWVRQAPGKGLEWVSSISGDSVTTYYADSVKGRFTI |
| | SRDNSKNTLYLQMNSLRAEDTAVYYCAKGPPTYHTNYYYMDVWGKGTTVTVSS |
| | HHHHHH (SEQ ID NO: 152) |
| CDA_(G4S)10 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYTFGGGTKVEIKGGGGSGGGGSGGGGSEVQLVESGGGLVQPGG |
| | SLRLSCAASGETFSDHYMDWVRQAPGKGLASGETFSDHYMDWVRQAPGKGLEW |
| | VGRIRNKASSYTTEYAASVKGRFTISRDDSKNSLYLQMNSLKTEDTAVYYCAREPK |
| | YWIDFDLWGRGTLVTVSSHHHHHH (SEQ ID NO: 153) |
| CDA_(G4S)10 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYTFGGGTKVEIKGGGGSGGGGSGGGGSEVQLVESGGGLVQPGR |
| | SLRLSCTASGFTFSDHDMNWVRQAPGKGLEWVGRTRNAAGSYTTEYAASVKGRF |
| | TISRDDSKNSLYLQMNSLKTEDTAVYYCAREPKYWIDFDLWGRGTLVTSSHHHHH |
| | H (SEQ ID NO: 154) |
| CDA_(G4S)10 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSEVQLVESGGGLVQPGGSLRLSCAA |
| | SGETFVDHDMDWVRQAPGKGLEWVGRTRNKLGSYTTEYAASVKGRETGRDDSK |
| | NSLYLQMNSLKTEDTAVYYCAREPKYWIDFDLWGRGTLVTVSSGGGGSGGGGSG |
| | GGGSDIQMTQSPSSLSASVGDRVTITCRASQSISSYLNWYQQKPGKAPKLLIYAASS |
| | LQSGVPSRFSGSGSGTDFTLTISSLQPEDFATYYCQQSYIAPYIFGGGTKVEIKHHHH |
| | HH (SEQ ID NO: 155) |
| CDA_(G4S)11 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGGGGGSGGGGSDIQMTQSPSSVSASVGDRV |
| | TITCRASQGISSWLAWYQQKPGKAPKLLLIYAASSLQSGVPSRFSGSGSGTDFTLTIS |
| | SLQPEDFATYYCQQTNSFPYTFGGGTKVEIKGGGGSGGGGSGGGGSEVQLLESGG |
| | GLVQPGGSLRLSCAASGETFSNYAMSWVRQAPGKGLEWVSAISGSGGSTYYADSV |
| | KGRFTISRDNSKNTLYLQMNSLRAEDTAVYYCAKGPPTYHTNYYYMDVWGKGTT |
| | VTVSSHHHHHH (SEQ ID NO: 156) |
| CDA_(G4S)11 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSDIQMTQSPSSVSASVGDRV |
| | TITCRASQGISSWLAWYQQKPGKAPKLLIYAASSLQQSGVPSRFSGSGSGTDFTLTI |
| | SSLQPEDFATYYCQQTNSEPYTFGGGTKVEIKGGGGSGGGGSGGGGSEVQLLESG |
| | GGLVQPGGSLRLSCAASGETESSYVMIWVRQAPGKGLEWVSSISGDSVTTYYADS |
| | VKGRFTISRDNSKNTLYLQMNSLRAEDTAVYYCAKGPPTYHTNYYYMDVWGKG |
| | TTVTVSSHHHHHH (SEQ ID NO: 157) |
| CDA_(G4S)11 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYTFGGGTKVEIKGGGGSGGGGSGGGGSEVQLVESGGG |
| | LVQPGGSLRLSCAASGETFSDHYMDWVRQAPGKGLASGETFSDHYMDWVRQAP |
| | GKGLEWVGRIRNKASSYTTEYAASVKGRFTISRDDSKNSLYLQMNSLKTEDTAVY |
| | YCAREPKYWIDFDLWGRGTLVTVSSHHHHHH (SEQ ID NO: 158) |
| CDA_(G4S)11 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYTFGGGTKVEIKGGGGSGGGGSGGGGSEVQLVESGGG |
| | LVQPGRSLRLSCTASGFTFSDHDMNWVRQAPGKGLEWVGRTRNAAGSYTTEYAA |
| | SVKGRFTISRDDSKNSLYLQMNSLKTEDTAVYYCAREPKYWIDFDLWGRGTLVTS |
| | SHHHHHH (SEQ ID NO: 159) |
| CDA_(G4S)11 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGGGGGSGGGGSGGGGSGGGGSEVQLVESGGGLVQPGGSLR |
| | LSCAASGETFVDHDMDWVRQAPGKGLEWVGRTRNKLGSYTTEYAASVKGRETG |
| | RDDSKNSLYLQMNSLKTEDTAVYYCAREPKYWIDFDLWGRGTLVTVSSGGGGSG |
| | GGGSGGGGSDIQMTQSPSSLSASVGDRVTITCRASQSISSYLNWYQQKPGKAPKLLI |
| | YAASSLQSGVPSRFSGSGSGTDFTLTISSLQPEDFATYYCQQSYIAPYIFGGGTKVEI |
| | KHHHHHH (SEQ ID NO: 160) |
| CDA_(G4S)112 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSGGGGSDIQMTQSPSSVSAS |
| | VGDRVTITCRASQGISSWLAWYQQKPGKAPKLLLIYAASSLQSGVPSRFSGSGSGT |
| | DFTLTISSLQPEDFATYYCQQTNSFPYTFGGGTKVEIKGGGGSGGGGSGGGGSEVQ |
| | LLESGGGLVQPGGSLRLSCAASGETFSNYAMSWVRQAPGKGLEWVSAISGSGGST |
| | YYADSVKGRFTISRDNSKNTLYLQMNSLRAEDTAVYYCAKGPPTYHTNYYYMDV |
| | WGKGTTVTVSSHHHHHH (SEQ ID NO: 161) |
| CDA_(G4S)112 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSGGGGSDIQMTQSPSSVSAS |
| | VGDRVTITCRASQGISSWLAWYQQKPGKAPKLLIYAASSLQQSGVPSRFSGSGSGT |
| | DFTLTISSLQPEDFATYYCQQTNSEPYTFGGGTKVEIKGGGGSGGGGSGGGGSEVQ |
| | LLESGGGLVQPGGSLRLSCAASGETESSYVMIWVRQAPGKGLEWVSSISGDSVTTY |
| | YADSVKGRFTISRDNSKNTLYLQMNSLRAEDTAVYYCAKGPPTYHTNYYYMDVW |
| | GKGTTVTVSSHHHHHH (SEQ ID NO: 162) |
| CDA_(G4S)112 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYTFGGGTKVEIKGGGGSGGGGSGGGGSEVQLV |
| | ESGGGLVQPGGSLRLSCAASGETFSDHYMDWVRQAPGKGLASGETFSDHYMDWV |
| | RQAPGKGLEWVGRIRNKASSYTTEYAASVKGRFTISRDDSKNSLYLQMNSLKTED |
| | TAVYYCAREPKYWIDFDLWGRGTLVTVSSHHHHHH (SEQ ID NO: 163) |
| CDA_(G4S)112 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYTFGGGTKVEIKGGGGSGGGGSGGGGSEVQLV |
| | ESGGGLVQPGRSLRLSCTASGFTFSDHDMNWVRQAPGKGLEWVGRTRNAAGSYT |
| | TEYAASVKGRFTISRDDSKNSLYLQMNSLKTEDTAVYYCAREPKYWIDFDLWGRG |
| | TLVTSSHHHHHH (SEQ ID NO: 164) |
| CDA_(G4S)112 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC_HIS | GGGGSGGGGSGGGGSGGGGSGGGGSGGGGSGGGGSGGGGSEVQLVESGGGLVQ |
| | PGGSLRLSCAASGETFVDHDMDWVRQAPGKGLEWVGRTRNKLGSYTTEYAASVK |
| | GRETGRDDSKNSLYLQMNSLKTEDTAVYYCAREPKYWIDFDLWGRGTLVTVSSG |
| | GGGSGGGGSGGGGSDIQMTQSPSSLSASVGDRVTITCRASQSISSYLNWYQQKPGK |
| | APKLLIYAASSLQSGVPSRFSGSGSGTDFTLTISSLQPEDFATYYCQQSYIAPYIFGG |
| | GTKVEIKHHHHHH (SEQ ID NO: 165) |
| CDA_G4S | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLLESGGGLVQPGG |
| LC_HIS | SLRLSCAASGETFSNYAMSWVRQAPGKGLEWVSAISGSGGSTYYADSVKGRFTISR |
| | DNSKNTLYLQMNSLRAEDTAVYYCAKGPPTYHTNYYYMDVWGKGTTVTVSSGG |
| | GGSGGGGSGGGGSDIQMTQSPSSVSASVGDRVTITCRASQGISSWLAWYQQKPGK |
| | APKLLLIYAASSLQSGVPSRFSGSGSGTDFTLTISSLQPEDFATYYCQQTNSFPYTFG |
| | GGTKVEIKHHHHHH (SEQ ID NO: 166) |
| CDA_G4S | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLLESGGGLVQPGG |
| LC_HIS | SLRLSCAASGETESSYVMIWVRQAPGKGLEWVSSISGDSVTTYYADSVKGRFTISR |
| | DNSKNTLYLQMNSLRAEDTAVYYCAKGPPTYHTNYYYMDVWGKGTTVTVSSGG |
| | GGSGGGGSGGGGSDIQMTQSPSSVSASVGDRVTITCRASQGISSWLAWYQQKPGK |
| | APKLLIYAASSLQQSGVPSRFSGSGSGTDFTLTISSLQPEDFATYYCQQTNSEPYTFG |
| | GGTKVEIKHHHHHH (SEQ ID NO: 167) |
| CDA_G4S | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLVESGGGLVQPGG |
| LC_HIS | SLRLSCAASGETFSDHYMDWVRQAPGKGLASGETFSDHYMDWVRQAPGKGLEW |
| | VGRIRNKASSYTTEYAASVKGRFTISRDDSKNSLYLQMNSLKTEDTAVYYCAREPK |
| | YWIDFDLWGRGTLVTVSSGGGGSGGGGSGGGGSDIQMTQSPSSLSASVGDRVTIT |
| | CRASQSISSYLNWYQQKPGKAPKLLIYAASSLQSGVPSRFSGSGSGTDFTLTISSLQP |
| | EDFATYYCQQSYIAPYTFGGGTKVEIKHHHHHH (SEQ ID NO: 168) |
| CDA_G4S | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLVESGGGLVQPGR |
| LC_HIS | SLRLSCTASGFTFSDHDMNWVRQAPGKGLEWVGRTRNAAGSYTTEYAASVKGRF |
| | TISRDDSKNSLYLQMNSLKTEDTAVYYCAREPKYWIDFDLWGRGTLVTSSGGGGS |
| | GGGGGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIKHHHHHH (SEQ ID NO: 169) |
| CDA_G4S | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSDIQMTQSPSSLSASVG |
| LC_HIS | DRVTITCRASQSISSYLNWYQQKPGKAPKLLIYAASSLQSGVPSRFSGSGSGTDFTL |
| | TISSLQPEDFATYYCQQSYIAPYIFGGGTKVEIKGGGGSGGGGSGGGGSEVQLVESG |
| | GGLVQPGGSLRLSCAASGETFVDHDMDWVRQAPGKGLEWVGRTRNKLGSYTTE |
| | YAASVKGRETGRDDSKNSLYLQMNSLKTEDTAVYYCAREPKYWIDFDLWGRGTL |
| | VTVSSHHHHHH (SEQ ID NO: 170) |
| CDA_(G4S)3 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLLE |
| LC_HIS | SGGGLVQPGGSLRLSCAASGETFSNYAMSWVRQAPGKGLEWVSAISGSGGSTYYA |
| | DSVKGRFTISRDNSKNTLYLQMNSLRAEDTAVYYCAKGPPTYHTNYYYMDVWGK |
| | GTTVTVSSGGGGSGGGGSGGGGSDIQMTQSPSSVSASVGDRVTITCRASQGISSWL |
| | AWYQQKPGKAPKLLLIYAASSLQSGVPSRFSGSGSGTDFTLTISSLQPEDFATYYCQ |
| | QTNSFPYTFGGGTKVEIKHHHHHH (SEQ ID NO: 171) |
| CDA_(G4S)3 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLLE |
| LC_HIS | SGGGLVQPGGSLRLSCAASGETESSYVMIWVRQAPGKGLEWVSSISGDSVTTYYA |
| | DSVKGRFTISRDNSKNTLYLQMNSLRAEDTAVYYCAKGPPTYHTNYYYMDVWGK |
| | GTTVTVSSGGGGSGGGGSGGGGSDIQMTQSPSSVSASVGDRVTITCRASQGISSWL |
| | AWYQQKPGKAPKLLIYAASSLQQSGVPSRFSGSGSGTDFTLTISSLQPEDFATYYCQ |
| | QTNSEPYTFGGGTKVEIKHHHHHH (SEQ ID NO: 172) |
| CDA_(G4S)3 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLV |
| LC_HIS | ESGGGLVQPGGSLRLSCAASGETFSDHYMDWVRQAPGKGLASGETFSDHYMDWV |
| | RQAPGKGLEWVGRIRNKASSYTTEYAASVKGRFTISRDDSKNSLYLQMNSLKTED |
| | TAVYYCAREPKYWIDFDLWGRGTLVTVSSGGGGSGGGGSGGGGSDIQMTQSPSSL |
| | SASVGDRVTITCRASQSISSYLNWYQQKPGKAPKLLIYAASSLQSGVPSRFSGSGSG |
| | TDFTLTISSLQPEDFATYYCQQSYIAPYTFGGGTKVEIKHHHHHH (SEQ ID NO: 173) |
| CDA_(G4S)3 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLV |
| LC_HIS | ESGGGLVQPGRSLRLSCTASGFTFSDHDMNWVRQAPGKGLEWVGRTRNAAGSYT |
| | TEYAASVKGRFTISRDDSKNSLYLQMNSLKTEDTAVYYCAREPKYWIDFDLWGRG |
| | TLVTSSGGGGSGGGGSGGGGSDIQMTQSPSSLSASVGDRVTITCRASQSISSYLNW |
| | YQQKPGKAPKLLIYAASSLQSGVPSRFSGSGSGTDFTLTISSLQPEDFATYYCQQSYI |
| | APYTFGGGTKVEIKHHHHHH (SEQ ID NO: 174) |
| CDA_(G4S)3 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSDIQMT |
| LC_HIS | QSPSSLSASVGDRVTITCRASQSISSYLNWYQQKPGKAPKLLIYAASSLQSGVPSRFS |
| | GSGSGTDFTLTISSLQPEDFATYYCQQSYIAPYIFGGGTKVEIKGGGGSGGGGSGGG |
| | GSEVQLVESGGGLVQPGGSLRLSCAASGETFVDHDMDWVRQAPGKGLEWVGRTR |
| | NKLGSYTTEYAASVKGRETGRDDSKNSLYLQMNSLKTEDTAVYYCAREPKYWID |
| | FDLWGRGTLVTVSSHHHHHH (SEQ ID NO: 175) |
| CDA_(G4S)4 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | EVQLLESGGGLVQPGGSLRLSCAASGETFSNYAMSWVRQAPGKGLEWVSAISGSG |
| | GSTYYADSVKGRFTISRDNSKNTLYLQMNSLRAEDTAVYYCAKGPPTYHTNYYY |
| | MDVWGKGTTVTVSSGGGGSGGGGSGGGGSDIQMTQSPSSVSASVGDRVTITCRAS |
| | QGISSWLAWYQQKPGKAPKLLLIYAASSLQSGVPSRFSGSGSGTDFTLTISSLQPED |
| | FATYYCQQTNSFPYTFGGGTKVEIKHHHHHH (SEQ ID NO: 176) |
| CDA_(G4S)4 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGGGGGS |
| LC_HIS | EVQLLESGGGLVQPGGSLRLSCAASGETESSYVMIWVRQAPGKGLEWVSSISGDSV |
| | TTYYADSVKGRFTISRDNSKNTLYLQMNSLRAEDTAVYYCAKGPPTYHTNYYYM |
| | DVWGKGTTVTVSSGGGGSGGGGSGGGGSDIQMTQSPSSVSASVGDRVTITCRASQ |
| | GISSWLAWYQQKPGKAPKLLIYAASSLQQSGVPSRFSGSGSGTDFTLTISSLQPEDF |
| | ATYYCQQTNSEPYTFGGGTKVEIKHHHHHH (SEQ ID NO: 177) |
| CDA_(G4S)4 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | EVQLVESGGGLVQPGGSLRLSCAASGETFSDHYMDWVRQAPGKGLASGETFSDH |
| | YMDWVRQAPGKGLEWVGRIRNKASSYTTEYAASVKGRFTISRDDSKNSLYLQMN |
| | SLKTEDTAVYYCAREPKYWIDFDLWGRGTLVTVSSGGGGSGGGGSGGGGSDIQM |
| | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKHHHHHH (SEQ |
| | ID NO: 178) |
| CDA_(G4S)4 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGGGGGSGGGGSGGGGS |
| LC_HIS | EVQLVESGGGLVQPGRSLRLSCTASGFTFSDHDMNWVRQAPGKGLEWVGRTRNA |
| | AGSYTTEYAASVKGRFTISRDDSKNSLYLQMNSLKTEDTAVYYCAREPKYWIDFD |
| | LWGRGTLVTSSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIKHHHHHH (SEQ ID NO: 179) |
| CDA_(G4S)4 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYIFGGGTKVEIKGGGGSGGG |
| | GSGGGGSEVQLVESGGGLVQPGGSLRLSCAASGETFVDHDMDWVRQAPGKGLE |
| | WVGRTRNKLGSYTTEYAASVKGRETGRDDSKNSLYLQMNSLKTEDTAVYYCARE |
| | PKYWIDFDLWGRGTLVTVSSHHHHHH (SEQ ID NO: 180) |
| CDA_(G4S)5 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSEVQLLESGGGLVQPGGSLRLSCAASGETFSNYAMSWVRQAPGKGLEWVS |
| | AISGSGGSTYYADSVKGRFTISRDNSKNTLYLQMNSLRAEDTAVYYCAKGPPTYH |
| | TNYYYMDVWGKGTTVTVSSGGGGSGGGGSGGGGSDIQMTQSPSSVSASVGDRVT |
| | ITCRASQGISSWLAWYQQKPGKAPKLLLIYAASSLQSGVPSRFSGSGSGTDFTLTISS |
| | LQPEDFATYYCQQTNSFPYTFGGGTKVEIKHHHHHH (SEQ ID NO: 181) |
| CDA_(G4S)5 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSEVQLLESGGGLVQPGGSLRLSCAASGETESSYVMIWVRQAPGKGLEWVSS |
| | ISGDSVTTYYADSVKGRFTISRDNSKNTLYLQMNSLRAEDTAVYYCAKGPPTYHT |
| | NYYYMDVWGKGTTVTVSSGGGGSGGGGSGGGGSDIQMTQSPSSVSASVGDRVTI |
| | TCRASQGISSWLAWYQQKPGKAPKLLIYAASSLQQSGVPSRFSGSGSGTDFTLTISS |
| | LQPEDFATYYCQQTNSEPYTFGGGTKVEIKHHHHHH (SEQ ID NO: 182) |
| CDA_(G4S)5 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSEVQLVESGGGLVQPGGSLRLSCAASGETFSDHYMDWVRQAPGKGLASGE |
| | TFSDHYMDWVRQAPGKGLEWVGRIRNKASSYTTEYAASVKGRFTISRDDSKNSLY |
| | LQMNSLKTEDTAVYYCAREPKYWIDFDLWGRGTLVTVSSGGGGSGGGGSGGGGS |
| | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYTFGGGTKVEIKHHHHHH |
| | (SEQ ID NO: 183) |
| CDA_(G4S)5 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSEVQLVESGGGLVQPGRSLRLSCTASGFTFSDHDMNWVRQAPGKGLEWVG |
| | RTRNAAGSYTTEYAASVKGRFTISRDDSKNSLYLQMNSLKTEDTAVYYCAREPKY |
| | WIDFDLWGRGTLVTSSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYTFGGGTKVEIKHHHHHH (SEQ ID NO: 184) |
| CDA_(G4S)5 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYIFGGGTKVEIKGGG |
| | GSGGGGSGGGGSEVQLVESGGGLVQPGGSLRLSCAASGETFVDHDMDWVRQAPG |
| | KGLEWVGRTRNKLGSYTTEYAASVKGRETGRDDSKNSLYLQMNSLKTEDTAVYY |
| | CAREPKYWIDFDLWGRGTLVTVSSHHHHHH (SEQ ID NO: 185) |
| CDA_(G4S)6 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSEVQLLESGGGLVQPGGSLRLSCAASGETFSNYAMSWVRQAPGKG |
| | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSDIQMTQSPSSVSASV |
| | GDRVTITCRASQGISSWLAWYQQKPGKAPKLLLIYAASSLQSGVPSRFSGSGSGTD |
| | FTLTISSLQPEDFATYYCQQTNSFPYTFGGGTKVEIKHHHHHH (SEQ ID NO: 186) |
| CDA_(G4S)6 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSEVQLLESGGGLVQPGGSLRLSCAASGETESSYVMIWVRQAPGKGL |
| | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSDIQMTQSPSSVSASVG |
| | DRVTITCRASQGISSWLAWYQQKPGKAPKLLIYAASSLQQSGVPSRFSGSGSGTDF |
| | TLTISSLQPEDFATYYCQQTNSEPYTFGGGTKVEIKHHHHHH (SEQ ID NO: 187) |
| CDA_(G4S)6 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSEVQLVESGGGLVQPGGSLRLSCAASGETFSDHYMDWVRQAPGKG |
| | LASGETFSDHYMDWVRQAPGKGLEWVGRIRNKASSYTTEYAASVKGRFTISRDDS |
| | KNSLYLQMNSLKTEDTAVYYCAREPKYWIDFDLWGRGTLVTVSSGGGGSGGGGS |
| | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYTFGGGTKVEIKHHH |
| | HHH (SEQ ID NO: 188) |
| CDA_(G4S)6 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSEVQLVESGGGLVQPGRSLRLSCTASGFTFSDHDMNWVRQAPGKG |
| | LEWVGRTRNAAGSYTTEYAASVKGRFTISRDDSKNSLYLQMNSLKTEDTAVYYC |
| | AREPKYWIDFDLWGRGTLVTSSGGGGSGGGGSGGGGSDIQMTQSPSSLSASVGDR |
| | VTITCRASQSISSYLNWYQQKPGKAPKLLIYAASSLQSGVPSRFSGSGSGTDFTLTIS |
| | SLQPEDFATYYCQQSYIAPYTFGGGTKVEIKHHHHHH (SEQ ID NO: 189) |
| CDA_(G4S)6 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYIFGGGTKV |
| | EIKGGGGSGGGGSGGGGSEVQLVESGGGLVQPGGSLRLSCAASGETFVDHDMDW |
| | VRQAPGKGLEWVGRTRNKLGSYTTEYAASVKGRETGRDDSKNSLYLQMNSLKTE |
| | DTAVYYCAREPKYWIDFDLWGRGTLVTVSSHHHHHH (SEQ ID NO: 190) |
| CDA_(G4S)7 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSEVQLLESGGGLVQPGGSLRLSCAASGETFSNYAMSWVRQ |
| | APGKGLEWVSAISGSGGSTYYADSVKGRFTISRDNSKNTLYLQMNSLRAEDTAVY |
| | YCAKGPPTYHTNYYYMDVWGKGTTVTVSSGGGGSGGGGSGGGGSDIQMTQSPSS |
| | VSASVGDRVTITCRASQGISSWLAWYQQKPGKAPKLLLIYAASSLQSGVPSRFSGS |
| | GSGTDFTLTISSLQPEDFATYYCQQTNSFPYTFGGGTKVEIKHHHHHH (SEQ ID NO: |
| | 191) |
| CDA_(G4S)7 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSEVQLLESGGGLVQPGGSLRLSCAASGETESSYVMIWVRQA |
| | PGKGLEWVSSISGDSVTTYYADSVKGRFTISRDNSKNTLYLQMNSLRAEDTAVYY |
| | CAKGPPTYHTNYYYMDVWGKGTTVTVSSGGGGSGGGGSGGGGSDIQMTQSPSSV |
| | SASVGDRVTITCRASQGISSWLAWYQQKPGKAPKLLIYAASSLQQSGVPSRFSGSG |
| | SGTDFTLTISSLQPEDFATYYCQQTNSEPYTFGGGTKVEIKHHHHHH (SEQ ID NO: |
| | 192) |
| CDA_(G4S)7 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSEVQLVESGGGLVQPGGSLRLSCAASGETFSDHYMDWVRQ |
| | APGKGLASGETFSDHYMDWVRQAPGKGLEWVGRIRNKASSYTTEYAASVKGRFT |
| | ISRDDSKNSLYLQMNSLKTEDTAVYYCAREPKYWIDFDLWGRGTLVTVSSGGGGS |
| | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIKHHHHHH (SEQ ID NO: 193) |
| CDA_(G4S)7 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSEVQLVESGGGLVQPGRSLRLSCTASGFTFSDHDMNWVRQ |
| | APGKGLEWVGRTRNAAGSYTTEYAASVKGRFTISRDDSKNSLYLQMNSLKTEDTA |
| | VYYCAREPKYWIDFDLWGRGTLVTSSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYTFGGGTKVEIKHHHHHH (SEQ ID NO: 194) |
| CDA_(G4S)7 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSDIQMTQSPSSLSASVGDRVTITCRASQSISSYLNWYQQKPG |
| | KAPKLLIYAASSLQSGVPSRFSGSGSGTDFTLTISSLQPEDFATYYCQQSYIAPYIFG |
| | GGTKVEIKGGGGGGGGSGGGGSEVQLVESGGGLVQPGGSLRLSCAASGETFVDH |
| | DMDWVRQAPGKGLEWVGRTRNKLGSYTTEYAASVKGRETGRDDSKNSLYLQMN |
| | SLKTEDTAVYYCAREPKYWIDFDLWGRGTLVTVSSHHHHHH (SEQ ID NO: 195) |
| CDA_(G4S)8 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSEVQLLESGGGLVQPGGSLRLSCAASGETFSNYAMS |
| | WVRQAPGKGLEWVSAISGSGGSTYYADSVKGRFTISRDNSKNTLYLQMNSLRAED |
| | TAVYYCAKGPPTYHTNYYYMDVWGKGTTVTVSSGGGGSGGGGSGGGGSDIQMT |
| | QSPSSVSASVGDRVTITCRASQGISSWLAWYQQKPGKAPKLLLIYAASSLQSGVPSR |
| | FSGSGSGTDFTLTISSLQPEDFATYYCQQTNSFPYTFGGGTKVEIKHHHHHH (SEQ |
| | ID NO: 196) |
| CDA_(G4S)8 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSEVQLLESGGGLVQPGGSLRLSCAASGETESSYVMI |
| | WVRQAPGKGLEWVSSISGDSVTTYYADSVKGRFTISRDNSKNTLYLQMNSLRAED |
| | TAVYYCAKGPPTYHTNYYYMDVWGKGTTVTVSSGGGGGGGGSGGGGSDIQMT |
| | QSPSSVSASVGDRVTITCRASQGISSWLAWYQQKPGKAPKLLIYAASSLQQSGVPS |
| | RFSGSGSGTDFTLTISSLQPEDFATYYCQQTNSEPYTFGGGTKVEIKHHHHHH (SEQ |
| | ID NO: 197) |
| CDA_(G4S)8 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSEVQLVESGGGLVQPGGSLRLSCAASGETFSDHYM |
| | DWVRQAPGKGLASGETFSDHYMDWVRQAPGKGLEWVGRIRNKASSYTTEYAAS |
| | VKGRFTISRDDSKNSLYLQMNSLKTEDTAVYYCAREPKYWIDFDLWGRGTLVTVS |
| | SGGGGSGGGGSGGGGSDIQMTQSPSSLSASVGDRVTITCRASQSISSYLNWYQQKP |
| | GKAPKLLIYAASSLQSGVPSRFSGSGSGTDFTLTISSLQPEDFATYYCQQSYIAPYTF |
| | GGGTKVEIKHHHHHH (SEQ ID NO: 198) |
| CDA_(G4S)8 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSEVQLVESGGGLVQPGRSLRLSCTASGFTFSDHDM |
| | NWVRQAPGKGLEWVGRTRNAAGSYTTEYAASVKGRFTISRDDSKNSLYLQMNSL |
| | KTEDTAVYYCAREPKYWIDFDLWGRGTLVTSSGGGGSGGGGSGGGGSDIQMTQS |
| | PSSLSASVGDRVTITCRASQSISSYLNWYQQKPGKAPKLLIYAASSLQSGVPSRFSGS |
| | GSGTDFTLTISSLQPEDFATYYCQQSYIAPYTFGGGTKVEIKHHHHHH (SEQ ID NO: |
| | 199) |
| CDA_(G4S)8 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYIFGGGTKVEIKGGGGSGGGGSGGGGSEVQLVESGGGLVQPGGSLRLSCAASGET |
| | FVDHDMDWVRQAPGKGLEWVGRTRNKLGSYTTEYAASVKGRETGRDDSKNSLY |
| | LQMNSLKTEDTAVYYCAREPKYWIDFDLWGRGTLVTVSSHHHHHH (SEQ ID NO: |
| | 200) |
| CDA_(G4S)9 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSEVQLLESGGGLVQPGGSLRLSCAASGETFS |
| | NYAMSWVRQAPGKGLEWVSAISGSGGSTYYADSVKGRFTISRDNSKNTLYLQMN |
| | SLRAEDTAVYYCAKGPPTYHTNYYYMDVWGKGTTVTVSSGGGGSGGGGSGGGG |
| | SDIQMTQSPSSVSASVGDRVTITCRASQGISSWLAWYQQKPGKAPKLLLIYAASSL |
| | QSGVPSRFSGSGSGTDFTLTISSLQPEDFATYYCQQTNSFPYTFGGGTKVEIKHHHH |
| | HH (SEQ ID NO: 201) |
| CDA_(G4S)9 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSEVQLLESGGGLVQPGGSLRLSCAASGETES |
| | SYVMIWVRQAPGKGLEWVSSISGDSVTTYYADSVKGRFTISRDNSKNTLYLQMNS |
| | LRAEDTAVYYCAKGPPTYHTNYYYMDVWGKGTTVTVSSGGGGSGGGGSGGGGS |
| | DIQMTQSPSSVSASVGDRVTITCRASQGISSWLAWYQQKPGKAPKLLIYAASSLQQ |
| | SGVPSRFSGSGSGTDFTLTISSLQPEDFATYYCQQTNSEPYTFGGGTKVEIKHHHHH |
| | H (SEQ ID NO: 202) |
| CDA_(G4S)9 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSEVQLVESGGGLVQPGGSLRLSCAASGETFS |
| | DHYMDWVRQAPGKGLASGETFSDHYMDWVRQAPGKGLEWVGRIRNKASSYTTE |
| | YAASVKGRFTISRDDSKNSLYLQMNSLKTEDTAVYYCAREPKYWIDFDLWGRGTL |
| | VTVSSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYTFGGGTKVEIKHHHHHH (SEQ ID NO: 203) |
| CDA_(G4S)9 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSEVQLVESGGGLVQPGRSLRLSCTASGFTFS |
| | DHDMNWVRQAPGKGLEWVGRTRNAAGSYTTEYAASVKGRFTISRDDSKNSLYLQ |
| | MNSLKTEDTAVYYCAREPKYWIDFDLWGRGTLVTSSGGGGSGGGGSGGGGSDIQ |
| | MTQSPSSLSASVGDRVTITCRASQSISSYLNWYQQKPGKAPKLLIYAASSLQSGVPS |
| | RFSGSGSGTDFTLTISSLQPEDFATYYCQQSYIAPYTFGGGTKVEIKHHHHHH (SEQ |
| | ID NO: 204) |
| CDA_(G4S)9 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYIFGGGTKVEIKGGGGSGGGGSGGGGSEVQLVESGGGLVQPGGSLRLSC |
| | AASGETFVDHDMDWVRQAPGKGLEWVGRTRNKLGSYTTEYAASVKGRETGRDD |
| | SKNSLYLQMNSLKTEDTAVYYCAREPKYWIDFDLWGRGTLVTVSSHHHHHH |
| | (SEQ ID NO: 205) |
| CDA_(G4S)10 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSEVQLLESGGGLVQPGGSLRLSCAA |
| | SGETFSNYAMSWVRQAPGKGLEWVSAISGSGGSTYYADSVKGRFTISRDNSKNTL |
| | YLQMNSLRAEDTAVYYCAKGPPTYHTNYYYMDVWGKGTTVTVSSGGGGSGGGG |
| | SGGGGSDIQMTQSPSSVSASVGDRVTITCRASQGISSWLAWYQQKPGKAPKLLLIY |
| | AASSLQSGVPSRFSGSGSGTDFTLTISSLQPEDFATYYCQQTNSFPYTFGGGTKVEIK |
| | HHHHHH (SEQ ID NO: 206) |
| CDA_(G4S)10 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSEVQLLESGGGLVQPGGSLRLSCAA |
| | SGETESSYVMIWVRQAPGKGLEWVSSISGDSVTTYYADSVKGRFTISRDNSKNTLY |
| | LQMNSLRAEDTAVYYCAKGPPTYHTNYYYMDVWGKGTTVTVSSGGGGSGGGGS |
| | GGGGSDIQMTQSPSSVSASVGDRVTITCRASQGISSWLAWYQQKPGKAPKLLIYAA |
| | SSLQQSGVPSRFSGSGSGTDFTLTISSLQPEDFATYYCQQTNSEPYTFGGGTKVEIKH |
| | HHHHH (SEQ ID NO: 207) |
| CDA_(G4S)10 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGGGGGSGGGGSGGGGS |
| LC_HIS | GGGGSGGGGSGGGGGGGGSGGGGSGGGGSEVQLVESGGGLVQPGGSLRLSCAA |
| | SGETFSDHYMDWVRQAPGKGLASGETFSDHYMDWVRQAPGKGLEWVGRIRNKA |
| | SSYTTEYAASVKGRFTISRDDSKNSLYLQMNSLKTEDTAVYYCAREPKYWIDFDL |
| | WGRGTLVTVSSGGGGGGGGSGGGGSDIQMTQSPSSLSASVGDRVTITCRASQSIS |
| | SYLNWYQQKPGKAPKLLIYAASSLQSGVPSRFSGSGSGTDFTLTISSLQPEDFATYY |
| | CQQSYIAPYTFGGGTKVEIKHHHHHH (SEQ ID NO: 208) |
| CDA_(G4S)10 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSEVQLVESGGGLVQPGRSLRLSCTA |
| | SGFTFSDHDMNWVRQAPGKGLEWVGRTRNAAGSYTTEYAASVKGRFTISRDDSK |
| | NSLYLQMNSLKTEDTAVYYCAREPKYWIDFDLWGRGTLVTSSGGGGSGGGGSGG |
| | GGSDIQMTQSPSSLSASVGDRVTITCRASQSISSYLNWYQQKPGKAPKLLIYAASSL |
| | QSGVPSRFSGSGSGTDFTLTISSLQPEDFATYYCQQSYIAPYTFGGGTKVEIKHHHH |
| | HH (SEQ ID NO: 209) |
| CDA_(G4S)10 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYIFGGGTKVEIKGGGGSGGGGSGGGGSEVQLVESGGGLVQPGG |
| | SLRLSCAASGETFVDHDMDWVRQAPGKGLEWVGRTRNKLGSYTTEYAASVKGRE |
| | TGRDDSKNSLYLQMNSLKTEDTAVYYCAREPKYWIDFDLWGRGTLVTVSSHHHH |
| | HH (SEQ ID NO: 210) |
| CDA_(G4S)11 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSEVQLLESGGGLVQPGGSLR |
| | LSCAASGETFSNYAMSWVRQAPGKGLEWVSAISGSGGSTYYADSVKGRFTISRDN |
| | SKNTLYLQMNSLRAEDTAVYYCAKGPPTYHTNYYYMDVWGKGTTVTVSSGGGG |
| | SGGGGSGGGGSDIQMTQSPSSVSASVGDRVTITCRASQGISSWLAWYQQKPGKAP |
| | KLLLIYAASSLQSGVPSRFSGSGSGTDFTLTISSLQPEDFATYYCQQTNSFPYTFGGG |
| | TKVEIKHHHHHH (SEQ ID NO: 211) |
| CDA_(G4S)11 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSEVQLLESGGGLVQPGGSLR |
| | LSCAASGETESSYVMIWVRQAPGKGLEWVSSISGDSVTTYYADSVKGRFTISRDNS |
| | KNTLYLQMNSLRAEDTAVYYCAKGPPTYHTNYYYMDVWGKGTTVTVSSGGGGS |
| | GGGGSGGGGSDIQMTQSPSSVSASVGDRVTITCRASQGISSWLAWYQQKPGKAPK |
| | LLIYAASSLQQSGVPSRFSGSGSGTDFTLTISSLQPEDFATYYCQQTNSEPYTFGGGT |
| | KVEIKHHHHHH (SEQ ID NO: 212) |
| CDA_(G4S)11 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSEVQLVESGGGLVQPGGSLR |
| | LSCAASGETFSDHYMDWVRQAPGKGLASGETFSDHYMDWVRQAPGKGLEWVGR |
| | IRNKASSYTTEYAASVKGRFTISRDDSKNSLYLQMNSLKTEDTAVYYCAREPKYWI |
| | DFDLWGRGTLVTVSSGGGGSGGGGSGGGGSDIQMTQSPSSLSASVGDRVTITCRAS |
| | QSISSYLNWYQQKPGKAPKLLIYAASSLQSGVPSRFSGSGSGTDFTLTISSLQPEDFA |
| | TYYCQQSYIAPYTFGGGTKVEIKHHHHHH (SEQ ID NO: 213) |
| CDA_(G4S)11 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSEVQLVESGGGLVQPGRSLR |
| | LSCTASGFTFSDHDMNWVRQAPGKGLEWVGRTRNAAGSYTTEYAASVKGRFTIS |
| | RDDSKNSLYLQMNSLKTEDTAVYYCAREPKYWIDFDLWGRGTLVTSSGGGGSGG |
| | GGSGGGGSDIQMTQSPSSLSASVGDRVTITCRASQSISSYLNWYQQKPGKAPKLLIY |
| | AASSLQSGVPSRFSGSGSGTDFTLTISSLQPEDFATYYCQQSYIAPYTFGGGTKVEIK |
| | HHHHHH (SEQ ID NO: 214) |
| CDA_(G4S)11 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYIFGGGTKVEIKGGGGSGGGGSGGGGSEVQLVESGGGL |
| | VQPGGSLRLSCAASGETFVDHDMDWVRQAPGKGLEWVGRTRNKLGSYTTEYAAS |
| | VKGRETGRDDSKNSLYLQMNSLKTEDTAVYYCAREPKYWIDFDLWGRGTLVTVS |
| | SHHHHHH (SEQ ID NO: 215) |
| CDA_(G4S)112 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSGGGGSEVQLLESGGGLVQP |
| | GGSLRLSCAASGETFSNYAMSWVRQAPGKGLEWVSAISGSGGSTYYADSVKGRFT |
| | ISRDNSKNTLYLQMNSLRAEDTAVYYCAKGPPTYHTNYYYMDVWGKGTTVTVSS |
| | GGGGSGGGGSGGGGSDIQMTQSPSSVSASVGDRVTITCRASQGISSWLAWYQQKP |
| | GKAPKLLLIYAASSLQSGVPSRFSGSGSGTDFTLTISSLQPEDFATYYCQQTNSFPYT |
| | FGGGTKVEIKHHHHHH (SEQ ID NO: 216) |
| CDA_(G4S)112 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSGGGGSEVQLLESGGGLVQP |
| | GGSLRLSCAASGETESSYVMIWVRQAPGKGLEWVSSISGDSVTTYYADSVKGRFTI |
| | SRDNSKNTLYLQMNSLRAEDTAVYYCAKGPPTYHTNYYYMDVWGKGTTVTVSS |
| | GGGGSGGGGSGGGGSDIQMTQSPSSVSASVGDRVTITCRASQGISSWLAWYQQKP |
| | GKAPKLLIYAASSLQQSGVPSRFSGSGSGTDFTLTISSLQPEDFATYYCQQTNSEPYT |
| | FGGGTKVEIKHHHHHH (SEQ ID NO: 217) |
| CDA_(G4S)112 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSGGGGSEVQLVESGGGLVQ |
| | PGGSLRLSCAASGETFSDHYMDWVRQAPGKGLASGETFSDHYMDWVRQAPGKG |
| | LEWVGRIRNKASSYTTEYAASVKGRFTISRDDSKNSLYLQMNSLKTEDTAVYYCA |
| | REPKYWIDFDLWGRGTLVTVSSGGGGSGGGGSGGGGSDIQMTQSPSSLSASVGDR |
| | VTITCRASQSISSYLNWYQQKPGKAPKLLIYAASSLQSGVPSRFSGSGSGTDFTLTIS |
| | SLQPEDFATYYCQQSYIAPYTFGGGTKVEIKHHHHHH (SEQ ID NO: 218) |
| CDA_(G4S)112 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSGGGGSEVQLVESGGGLVQ |
| | PGRSLRLSCTASGFTFSDHDMNWVRQAPGKGLEWVGRTRNAAGSYTTEYAASVK |
| | GRFTISRDDSKNSLYLQMNSLKTEDTAVYYCAREPKYWIDFDLWGRGTLVTSSGG |
| | GGSGGGGSGGGGSDIQMTQSPSSLSASVGDRVTITCRASQSISSYLNWYQQKPGKA |
| | PKLLIYAASSLQSGVPSRFSGSGSGTDFTLTISSLQPEDFATYYCQQSYIAPYTFGGG |
| | TKVEIKHHHHHH (SEQ ID NO: 219) |
| CDA_(G4S)112 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC_HIS | GGGGSGGGGSGGGGSGGGGSGGGGSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYIFGGGTKVEIKGGGGSGGGGSGGGGSEVQLVE |
| | SGGGLVQPGGSLRLSCAASGETFVDHDMDWVRQAPGKGLEWVGRTRNKLGSYTT |
| | EYAASVKGRETGRDDSKNSLYLQMNSLKTEDTAVYYCAREPKYWIDFDLWGRGT |
| | LVTVSSHHHHHH (SEQ ID NO: 220) |
| Ab58_LC_ | DIQMTQSPSSVSASVGDRVTITCRASQGISSWLAWYQQKPGKAPKLLLIYAASSLQ |
| (G4S)3_HC_G4S | SGVPSRFSGSGSGTDFTLTISSLQPEDFATYYCQQTNSFPYTFGGGTKVEIKGGGGS |
| linker_CDA_ | GGGGSGGGGSEVQLLESGGGLVQPGGSLRLSCAASGETFSNYAMSWVRQAPGKG |
| | LEWVSAISGSGGSTYYADSVKGRFTISRDNSKNTLYLQMNSLRAEDTAVYYCAKG |
| | PPTYHTNYYYMDVWGKGTTVTVSSGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQ (SEQ ID NO: 221) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)3 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQ (SEQ ID NO: 222) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)4 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQ (SEQ ID NO: 223) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)5 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 224) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)6 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQ (SEQ ID NO: 225) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)7 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQ (SEQ ID NO: 226) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)8 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 227) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)9 linker_ | GGGGSGGGGSEVQLLESGGGLVQPGGSLRLSCAASGETFSNYAMSWVRQAPGKG |
| CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQ (SEQ |
| | ID NO: 228) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)10 | GGGGSGGGGSEVQLLESGGGLVQPGGSLRLSCAASGETFSNYAMSWVRQAPGKG |
| linker_CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | Q (SEQ ID NO: 229) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)11 | GGGGSGGGGSEVQLLESGGGLVQPGGSLRLSCAASGETFSNYAMSWVRQAPGKG |
| linker_CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQ (SEQ ID NO: 230) |
| Ab58_LC_ | DIQMTQSPSSVSASVGDRVTITCRASQGISSWLAWYQQKPGKAPKLLLIYAASSLQ |
| (G4S)3_HC_ | SGVPSRFSGSGSGTDFTLTISSLQPEDFATYYCQQTNSFPYTFGGGTKVEIKGGGGS |
| (G4S)112 | GGGGSGGGGSEVQLLESGGGLVQPGGSLRLSCAASGETFSNYAMSWVRQAPGKG |
| linker_CDA | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSGGGGSGGGGSGGGG |
| | SGGGGSGGGGSGGGGSGGGGSGGGGSGGGGSMAQKRPACTLKPECVQQLLVCSQ |
| | EAKKSAYCPYSHFPVGAALLTQEGRIFKGCNIENACYPLGICAERTAIQKAVSEGY |
| | KDFRAIAIASDMQDDFISPCGACRQVMREFGTNWPVYMTKPDGTYIVMTVQELLP |
| | SSFGPEDLQKTQ (SEQ ID NO: 231) |
| Ab61_LC_ | DIQMTQSPSSVSASVGDRVTITCRASQGISSWLAWYQQKPGKAPKLLIYAASSLQQ |
| (G4S)3_HC_G4S | SGVPSRFSGSGSGTDFTLTISSLQPEDFATYYCQQTNSEPYTFGGGTKVEIKGGGGS |
| linker_CDA | GGGGSGGGGSEVQLLESGGGLVQPGGSLRLSCAASGETESSYVMIWVRQAPGKGL |
| | EWVSSISGDSVTTYYADSVKGRFTISRDNSKNTLYLQMNSLRAEDTAVYYCAKGP |
| | PTYHTNYYYMDVWGKGTTVTVSSGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQ (SEQ ID NO: 232) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)3 linker_ | GGGGSGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQ (SEQ ID NO: 233) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)4 linker_ | GGGGSGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQ (SEQ ID NO: 234) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)5 linker_ | GGGGSGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 235) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)6 linker_ | GGGGSGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | MAQKRPACTLKPECVQQLLVCSQEAKKSAYCPYSHFPVGAALLTQEGRIFKGCNIE |
| | NACYPLGICAERTAIQKAVSEGYKDFRAIAIASDMQDDFISPCGACRQVMREFGTN |
| | WPVYMTKPDGTYIVMTVQELLPSSFGPEDLQKTQ (SEQ ID NO: 236) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)7 linker_ | GGGGSGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | GGGGSMAQKRPACTLKPECVQQLLVCSQEAKKSAYCPYSHFPVGAALLTQEGRIF |
| | KGCNIENACYPLGICAERTAIQKAVSEGYKDFRAIAIASDMQDDFISPCGACRQVM |
| | REFGTNWPVYMTKPDGTYIVMTVQELLPSSFGPEDLQKTQ (SEQ ID NO: 237) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)8 linker_ | GGGGGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | GGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 238) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)9 linker_ | GGGGSGGGGSEVQLLESGGGLVQPGGSLRLSCAASGETESSYVMIWVRQAPGKGL |
| CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | GGGGSGGGGSGGGGSMAQKRPACTLKPECVQQLLVCSQEAKKSAYCPYSHFPVG |
| | AALLTQEGRIFKGCNIENACYPLGICAERTAIQKAVSEGYKDFRAIAIASDMQDDFI |
| | SPCGACRQVMREFGTNWPVYMTKPDGTYIVMTVQELLPSSFGPEDLQKTQ (SEQ |
| | ID NO: 239) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)10 | GGGGSGGGGSEVQLLESGGGLVQPGGSLRLSCAASGETESSYVMIWVRQAPGKGL |
| linker_CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | GGGGSGGGGSGGGGSGGGGSMAQKRPACTLKPECVQQLLVCSQEAKKSAYCPYS |
| | HFPVGAALLTQEGRIFKGCNIENACYPLGICAERTAIQKAVSEGYKDFRAIAIASDM |
| | QDDFISPCGACRQVMREFGTNWPVYMTKPDGTYIVMTVQELLPSSFGPEDLQKTQ |
| | (SEQ ID NO: 240) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)11 | GGGGSGGGGSEVQLLESGGGLVQPGGSLRLSCAASGETESSYVMIWVRQAPGKGL |
| linker_CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | GGGGSGGGGSGGGGSGGGGSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQ (SEQ ID NO: 241) |
| Ab61_LC_ | DIQMTQSPSSVSASVGDRVTITCRASQGISSWLAWYQQKPGKAPKLLIYAASSLQQ |
| (G4S)3_HC_ | SGVPSRFSGSGSGTDFTLTISSLQPEDFATYYCQQTNSEPYTFGGGTKVEIKGGGGS |
| (G4S)112 | GGGGSGGGGSEVQLLESGGGLVQPGGSLRLSCAASGETESSYVMIWVRQAPGKGL |
| linker_CDA | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSGGGGSGGGGSGGGGS |
| | GGGGSGGGGSGGGGSGGGGSGGGGSGGGGSMAQKRPACTLKPECVQQLLVCSQE |
| | AKKSAYCPYSHFPVGAALLTQEGRIFKGCNIENACYPLGICAERTAIQKAVSEGYK |
| | DFRAIAIASDMQDDFISPCGACRQVMREFGTNWPVYMTKPDGTYIVMTVQELLPSS |
| | FGPEDLQKTQ (SEQ ID NO: 242) |
| Ab66_LC_ | DIQMTQSPSSLSASVGDRVTITCRASQSISSYLNWYQQKPGKAPKLLIYAASSLQSG |
| (G4S)3_HC_G4S | VPSRFSGSGSGTDFTLTISSLQPEDFATYYCQQSYIAPYTFGGGTKVEIKGGGGSGG |
| linker_CDA | GGSGGGGSEVQLVESGGGLVQPGGSLRLSCAASGETFSDHYMDWVRQAPGKGLA |
| | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSMAQKRPA |
| | CTLKPECVQQLLVCSQEAKKSAYCPYSHFPVGAALLTQEGRIFKGCNIENACYPLGI |
| | CAERTAIQKAVSEGYKDFRAIAIASDMQDDFISPCGACRQVMREFGTNWPVYMTK |
| | PDGTYIVMTVQELLPSSFGPEDLQKTQ (SEQ ID NO: 243) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)3 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSMAQKRPACTLKPECVQQLLVCSQEAKKSAYCPYSHFPVGAALLTQEGRIFKG |
| | CNIENACYPLGICAERTAIQKAVSEGYKDFRAIAIASDMQDDFISPCGACRQVMREF |
| | GTNWPVYMTKPDGTYIVMTVQELLPSSFGPEDLQKTQ (SEQ ID NO: 244) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)4 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSMAQKRPACTLKPECVQQLLVCSQEAKKSAYCPYSHFPVGAALLTQE |
| | GRIFKGCNIENACYPLGICAERTAIQKAVSEGYKDFRAIAIASDMQDDFISPCGACR |
| | QVMREFGTNWPVYMTKPDGTYIVMTVQELLPSSFGPEDLQKTQ (SEQ ID NO: 245) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)5 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSMAQKRPACTLKPECVQQLLVCSQEAKKSAYCPYSHFPVGAA |
| | LLTQEGRIFKGCNIENACYPLGICAERTAIQKAVSEGYKDFRAIAIASDMQDDFISPC |
| | GACRQVMREFGTNWPVYMTKPDGTYIVMTVQELLPSSFGPEDLQKTQ (SEQ ID |
| | NO: 246) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)6 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSGGGGSMAQKRPACTLKPECVQQLLVCSQEAKKSAYCPYSHF |
| | PVGAALLTQEGRIFKGCNIENACYPLGICAERTAIQKAVSEGYKDFRAIAIASDMQD |
| | DFISPCGACRQVMREFGTNWPVYMTKPDGTYIVMTVQELLPSSFGPEDLQKTQ |
| | (SEQ ID NO: 247) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)7 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSGGGGSGGGGSMAQKRPACTLKPECVQQLLVCSQEAKKSAYC |
| | PYSHFPVGAALLTQEGRIFKGCNIENACYPLGICAERTAIQKAVSEGYKDFRAIAIAS |
| | DMQDDFISPCGACRQVMREFGTNWPVYMTKPDGTYIVMTVQELLPSSFGPEDLQK |
| | TQ (SEQ ID NO: 248) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)8 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGGGGGSGGGGSGGGGSGGGGSMAQKRPACTLKPECVQQLLVCSQEAK |
| | KSAYCPYSHFPVGAALLTQEGRIFKGCNIENACYPLGICAERTAIQKAVSEGYKDFR |
| | AIAIASDMQDDFISPCGACRQVMREFGTNWPVYMTKPDGTYIVMTVQELLPSSFGP |
| | EDLQKTQ (SEQ ID NO: 249) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)9 linker_ | GGSGGGGSEVQLVESGGGLVQPGGSLRLSCAASGETFSDHYMDWVRQAPGKGLA |
| CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSMAQKRPACTLKPECVQQLLV |
| | CSQEAKKSAYCPYSHFPVGAALLTQEGRIFKGCNIENACYPLGICAERTAIQKAVSE |
| | GYKDFRAIAIASDMQDDFISPCGACRQVMREFGTNWPVYMTKPDGTYIVMTVQEL |
| | LPSSFGPEDLQKTQ (SEQ ID NO: 250) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)10 | GGSGGGGSEVQLVESGGGLVQPGGSLRLSCAASGETFSDHYMDWVRQAPGKGLA |
| linker_CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQ (SEQ ID NO: 251) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)11 | GGSGGGGSEVQLVESGGGLVQPGGSLRLSCAASGETFSDHYMDWVRQAPGKGLA |
| linker_CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQ (SEQ ID NO: 252) |
| Ab66_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)112 | GGSGGGGSEVQLVESGGGLVQPGGSLRLSCAASGETFSDHYMDWVRQAPGKGLA |
| linker_CDA | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSSGGGGSGGGGSGG |
| | GGSGGGGSGGGGSGGGGSGGGGSGGGGSGGGGSGGGGSGGGGSGGGGSMAQKR |
| | PACTLKPECVQQLLVCSQEAKKSAYCPYSHFPVGAALLTQEGRIFKGCNIENACYP |
| | LGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 253) |
| Ab68_LC_ | DIQMTQSPSSLSASVGDRVTITCRASQSISSYLNWYQQKPGKAPKLLIYAASSLQSG |
| (G4S)3_HC_G4S | VPSRFSGSGSGTDFTLTISSLQPEDFATYYCQQSYIAPYTFGGGTKVEIKGGGGSGG |
| linker_CDA | GGSGGGGSEVQLVESGGGLVQPGRSLRLSCTASGFTFSDHDMNWVRQAPGKGLE |
| | WVGRTRNAAGSYTTEYAASVKGRFTISRDDSKNSLYLQMNSLKTEDTAVYYCAR |
| | EPKYWIDFDLWGRGTLVTSSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQ (SEQ ID NO: 254) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)3 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQ (SEQ ID NO: 255) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)4 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQ (SEQ ID NO: 256) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)5 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQ (SEQ ID NO: 257) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)6 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSMAQ |
| | KRPACTLKPECVQQLLVCSQEAKKSAYCPYSHFPVGAALLTQEGRIFKGCNIENAC |
| | YPLGICAERTAIQKAVSEGYKDFRAIAIASDMQDDFISPCGACRQVMREFGTNWPV |
| | YMTKPDGTYIVMTVQELLPSSFGPEDLQKTQ (SEQ ID NO: 258) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)7 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQ (SEQ ID NO: 259) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)8 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQ (SEQ ID NO: 260) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)9 linker_ | GGSGGGGSEVQLVESGGGLVQPGRSLRLSCTASGFTFSDHDMNWVRQAPGKGLE |
| CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 261) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)10 | GGSGGGGSEVQLVESGGGLVQPGRSLRLSCTASGFTFSDHDMNWVRQAPGKGLE |
| linker_CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQ (SEQ |
| | ID NO: 262) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)11 | GGSGGGGSEVQLVESGGGLVQPGRSLRLSCTASGFTFSDHDMNWVRQAPGKGLE |
| linker_CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | Q (SEQ ID NO: 263) |
| Ab68_LC_ | DIQMTQSPSSLSASVGDRVTITCRASQSISSYLNWYQQKPGKAPKLLIYAASSLQSG |
| (G4S)3_HC_ | VPSRFSGSGSGTDFTLTISSLQPEDFATYYCQQSYIAPYTFGGGTKVEIKGGGGSGG |
| (G4S)112 | GGSGGGGSEVQLVESGGGLVQPGRSLRLSCTASGFTFSDHDMNWVRQAPGKGLE |
| linker_CDA | WVGRTRNAAGSYTTEYAASVKGRFTISRDDSKNSLYLQMNSLKTEDTAVYYCAR |
| | EPKYWIDFDLWGRGTLVTSSGGGGSGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQ (SEQ ID NO: 264) |
| Ab69_LC_ | EVQLVESGGGLVQPGGSLRLSCAASGETFVDHDMDWVRQAPGKGLEWVGRTRN |
| (G4S)3_HC_G4S | KLGSYTTEYAASVKGRETGRDDSKNSLYLQMNSLKTEDTAVYYCAREPKYWIDF |
| linker_CDA | DLWGRGTLVTVSSGGGGSGGGGSGGGGSDIQMTQSPSSLSASVGDRVTITCRASQS |
| | ISSYLNWYQQKPGKAPKLLIYAASSLQSGVPSRFSGSGSGTDFTLTISSLQPEDFATY |
| | YCQQSYIAPYIFGGGTKVEIKGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQ (SEQ ID NO: 265) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)3 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQ (SEQ ID NO: 266) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)4 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQ (SEQ ID NO: 267) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)5 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQ (SEQ ID NO: 268) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)6 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSGGGGSMAQ |
| | KRPACTLKPECVQQLLVCSQEAKKSAYCPYSHFPVGAALLTQEGRIFKGCNIENAC |
| | YPLGICAERTAIQKAVSEGYKDFRAIAIASDMQDDFISPCGACRQVMREFGTNWPV |
| | YMTKPDGTYIVMTVQELLPSSFGPEDLQKTQ (SEQ ID NO: 269) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)7 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQ (SEQ ID NO: 270) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)8 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQ (SEQ ID NO: 271) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)9 linker_ | DLWGRGTLVTVSSGGGGSGGGGSGGGGSDIQMTQSPSSLSASVGDRVTITCRASQS |
| CDA | ISSYLNWYQQKPGKAPKLLIYAASSLQSGVPSRFSGSGSGTDFTLTISSLQPEDFATY |
| | YCQQSYIAPYIFGGGTKVEIKGGGGGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 272) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)10 | DLWGRGTLVTVSSGGGGSGGGGSGGGGSDIQMTQSPSSLSASVGDRVTITCRASQS |
| linker_CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQ (SEQ |
| | ID NO: 273 |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)11 | DLWGRGTLVTVSSGGGGSGGGGSGGGGSDIQMTQSPSSLSASVGDRVTITCRASQS |
| linker_CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSGGGGGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | Q (SEQ ID NO: 274) |
| Ab69_LC_ | EVQLVESGGGLVQPGGSLRLSCAASGETFVDHDMDWVRQAPGKGLEWVGRTRN |
| (G4S)3_HC_ | KLGSYTTEYAASVKGRETGRDDSKNSLYLQMNSLKTEDTAVYYCAREPKYWIDF |
| (G4S)112 | DLWGRGTLVTVSSGGGGSGGGGSGGGGSDIQMTQSPSSLSASVGDRVTITCRASQS |
| linker_CDA | ISSYLNWYQQKPGKAPKLLIYAASSLQSGVPSRFSGSGSGTDFTLTISSLQPEDFATY |
| | YCQQSYIAPYIFGGGTKVEIKGGGGSGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQ (SEQ ID NO: 275) |
| Ab58_HC_ | EVQLLESGGGLVQPGGSLRLSCAASGETFSNYAMSWVRQAPGKGLEWVSAISGSG |
| (G4S)3_LC_G4S | GSTYYADSVKGRFTISRDNSKNTLYLQMNSLRAEDTAVYYCAKGPPTYHTNYYY |
| linker_CDA | MDVWGKGTTVTVSSGGGGSGGGGSGGGGSDIQMTQSPSSVSASVGDRVTITCRAS |
| | QGISSWLAWYQQKPGKAPKLLLIYAASSLQSGVPSRFSGSGSGTDFTLTISSLQPED |
| | FATYYCQQTNSFPYTFGGGTKVEIKGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQ (SEQ ID NO: 276) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)3 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQ (SEQ ID NO: 277) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)4 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQ (SEQ ID NO: 278) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)5 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 279) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)6 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SMAQKRPACTLKPECVQQLLVCSQEAKKSAYCPYSHFPVGAALLTQEGRIFKGCNI |
| | ENACYPLGICAERTAIQKAVSEGYKDFRAIAIASDMQDDFISPCGACRQVMREFGT |
| | NWPVYMTKPDGTYIVMTVQELLPSSFGPEDLQKTQ (SEQ ID NO: 280) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)7 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SGGGGSMAQKRPACTLKPECVQQLLVCSQEAKKSAYCPYSHFPVGAALLTQEGRI |
| | FKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQ (SEQ ID NO: 281) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)8 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SGGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 282) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)9 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SGGGGSGGGGSGGGGSMAQKRPACTLKPECVQQLLVCSQEAKKSAYCPYSHFPV |
| | GAALLTQEGRIFKGCNIENACYPLGICAERTAIQKAVSEGYKDFRAIAIASDMQDDF |
| | ISPCGACRQVMREFGTNWPVYMTKPDGTYIVMTVQELLPSSFGPEDLQKTQ (SEQ |
| | ID NO: 283) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)10 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SGGGGSGGGGSGGGGSGGGGSMAQKRPACTLKPECVQQLLVCSQEAKKSAYCPY |
| | SHFPVGAALLTQEGRIFKGCNIENACYPLGICAERTAIQKAVSEGYKDFRAIAIASD |
| | MQDDFISPCGACRQVMREFGTNWPVYMTKPDGTYIVMTVQELLPSSFGPEDLQKT |
| | Q (SEQ ID NO: 284) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)11 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SGGGGSGGGGSGGGGSGGGGSGGGGSMAQKRPACTLKPECVQQLLVCSQEAKKS |
| | AYCPYSHFPVGAALLTQEGRIFKGCNIENACYPLGICAERTAIQKAVSEGYKDFRAI |
| | AIASDMQDDFISPCGACRQVMREFGTNWPVYMTKPDGTYIVMTVQELLPSSFGPE |
| | DLQKTQ (SEQ ID NO: 285) |
| Ab58_HC_ | EVQLLESGGGLVQPGGSLRLSCAASGETFSNYAMSWVRQAPGKGLEWVSAISGSG |
| (G4S)3_LC_ | GSTYYADSVKGRFTISRDNSKNTLYLQMNSLRAEDTAVYYCAKGPPTYHTNYYY |
| (G4S)112 | MDVWGKGTTVTVSSGGGGSGGGGSGGGGSDIQMTQSPSSVSASVGDRVTITCRAS |
| linker_CDA | QGISSWLAWYQQKPGKAPKLLLIYAASSLQSGVPSRFSGSGSGTDFTLTISSLQPED |
| | FATYYCQQTNSFPYTFGGGTKVEIKGGGGSGGGGSGGGGSGGGGSGGGGSGGGG |
| | SGGGGSGGGGSGGGGSGGGGSGGGGSGGGGSMAQKRPACTLKPECVQQLLVCSQ |
| | EAKKSAYCPYSHFPVGAALLTQEGRIFKGCNIENACYPLGICAERTAIQKAVSEGY |
| | KDFRAIAIASDMQDDFISPCGACRQVMREFGTNWPVYMTKPDGTYIVMTVQELLP |
| | SSFGPEDLQKTQ (SEQ ID NO: 286) |
| Ab61_HC_ | EVQLLESGGGLVQPGGSLRLSCAASGETESSYVMIWVRQAPGKGLEWVSSISGDSV |
| (G4S)3_LC_G4S | TTYYADSVKGRFTISRDNSKNTLYLQMNSLRAEDTAVYYCAKGPPTYHTNYYYM |
| linker_CDA | DVWGKGTTVTVSSGGGGSGGGGSGGGGSDIQMTQSPSSVSASVGDRVTITCRASQ |
| | GISSWLAWYQQKPGKAPKLLIYAASSLQQSGVPSRFSGSGSGTDFTLTISSLQPEDF |
| | ATYYCQQTNSEPYTFGGGTKVEIKGGGGSMAQKRPACTLKPECVQQLLVCSQEAK |
| | KSAYCPYSHFPVGAALLTQEGRIFKGCNIENACYPLGICAERTAIQKAVSEGYKDFR |
| | AIAIASDMQDDFISPCGACRQVMREFGTNWPVYMTKPDGTYIVMTVQELLPSSFGP |
| | EDLQKTQ (SEQ ID NO: 287) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)3 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSMAQKRPACTLKPECV |
| | QQLLVCSQEAKKSAYCPYSHFPVGAALLTQEGRIFKGCNIENACYPLGICAERTAIQ |
| | KAVSEGYKDFRAIAIASDMQDDFISPCGACRQVMREFGTNWPVYMTKPDGTYIVM |
| | TVQELLPSSFGPEDLQKTQ (SEQ ID NO: 288) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)4 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQ (SEQ ID NO: 289) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)5 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSMAQKR |
| | PACTLKPECVQQLLVCSQEAKKSAYCPYSHFPVGAALLTQEGRIFKGCNIENACYP |
| | LGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 290) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)6 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | MAQKRPACTLKPECVQQLLVCSQEAKKSAYCPYSHFPVGAALLTQEGRIFKGCNIE |
| | NACYPLGICAERTAIQKAVSEGYKDFRAIAIASDMQDDFISPCGACRQVMREFGTN |
| | WPVYMTKPDGTYIVMTVQELLPSSFGPEDLQKTQ (SEQ ID NO: 291) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)7 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | GGGGSMAQKRPACTLKPECVQQLLVCSQEAKKSAYCPYSHFPVGAALLTQEGRIF |
| | KGCNIENACYPLGICAERTAIQKAVSEGYKDFRAIAIASDMQDDFISPCGACRQVM |
| | REFGTNWPVYMTKPDGTYIVMTVQELLPSSFGPEDLQKTQ (SEQ ID NO: 292) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)8 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | GGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 293) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)9 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | GGGGSGGGGSGGGGSMAQKRPACTLKPECVQQLLVCSQEAKKSAYCPYSHFPVG |
| | AALLTQEGRIFKGCNIENACYPLGICAERTAIQKAVSEGYKDFRAIAIASDMQDDFI |
| | SPCGACRQVMREFGTNWPVYMTKPDGTYIVMTVQELLPSSFGPEDLQKTQ (SEQ |
| | ID NO: 294) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)10 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | GGGGSGGGGSGGGGSGGGGSMAQKRPACTLKPECVQQLLVCSQEAKKSAYCPYS |
| | HFPVGAALLTQEGRIFKGCNIENACYPLGICAERTAIQKAVSEGYKDFRAIAIASDM |
| | QDDFISPCGACRQVMREFGTNWPVYMTKPDGTYIVMTVQELLPSSFGPEDLQKTQ |
| | (SEQ ID NO: 295) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)11 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | GGGGSGGGGSGGGGSGGGGSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQ (SEQ ID NO: 296) |
| Ab61_HC_ | EVQLLESGGGLVQPGGSLRLSCAASGETESSYVMIWVRQAPGKGLEWVSSISGDSV |
| (G4S)3_LC_ | TTYYADSVKGRFTISRDNSKNTLYLQMNSLRAEDTAVYYCAKGPPTYHTNYYYM |
| (G4S)112 | DVWGKGTTVTVSSGGGGSGGGGSGGGGSDIQMTQSPSSVSASVGDRVTITCRASQ |
| linker_CDA | GISSWLAWYQQKPGKAPKLLIYAASSLQQSGVPSRFSGSGSGTDFTLTISSLQPEDF |
| | ATYYCQQTNSEPYTFGGGTKVEIKGGGGSGGGGSGGGGSGGGGSGGGGSGGGGS |
| | GGGGSGGGGSGGGGSGGGGSGGGGSGGGGSMAQKRPACTLKPECVQQLLVCSQE |
| | AKKSAYCPYSHFPVGAALLTQEGRIFKGCNIENACYPLGICAERTAIQKAVSEGYK |
| | DFRAIAIASDMQDDFISPCGACRQVMREFGTNWPVYMTKPDGTYIVMTVQELLPSS |
| | FGPEDLQKTQ (SEQ ID NO: 297) |
| Ab66_HC_ | EVQLVESGGGLVQPGGSLRLSCAASGETFSDHYMDWVRQAPGKGLASGETFSDH |
| (G4S)3_LC_G4S | YMDWVRQAPGKGLEWVGRIRNKASSYTTEYAASVKGRFTISRDDSKNSLYLQMN |
| linker_CDA | SLKTEDTAVYYCAREPKYWIDFDLWGRGTLVTVSSGGGGSGGGGSGGGGSDIQM |
| | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSMAQKRP |
| | ACTLKPECVQQLLVCSQEAKKSAYCPYSHFPVGAALLTQEGRIFKGCNIENACYPL |
| | GICAERTAIQKAVSEGYKDFRAIAIASDMQDDFISPCGACRQVMREFGTNWPVYM |
| | TKPDGTYIVMTVQELLPSSFGPEDLQKTQ (SEQ ID NO: 298) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)3 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSMAQKRPACTLKPECVQQLLVCSQEAKKSAYCPYSHFPVGAALLTQEGRIFK |
| | GCNIENACYPLGICAERTAIQKAVSEGYKDFRAIAIASDMQDDFISPCGACRQVMR |
| | EFGTNWPVYMTKPDGTYIVMTVQELLPSSFGPEDLQKTQ (SEQ ID NO: 299) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)4 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSMAQKRPACTLKPECVQQLLVCSQEAKKSAYCPYSHFPVGAALLTQ |
| | EGRIFKGCNIENACYPLGICAERTAIQKAVSEGYKDFRAIAIASDMQDDFISPCGAC |
| | RQVMREFGTNWPVYMTKPDGTYIVMTVQELLPSSFGPEDLQKTQ (SEQ ID NO: |
| | 300) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)5 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSMAQKRPACTLKPECVQQLLVCSQEAKKSAYCPYSHFPVGA |
| | ALLTQEGRIFKGCNIENACYPLGICAERTAIQKAVSEGYKDFRAIAIASDMQDDFISP |
| | CGACRQVMREFGTNWPVYMTKPDGTYIVMTVQELLPSSFGPEDLQKTQ (SEQ ID |
| | NO: 301) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)6 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSMAQKRPACTLKPECVQQLLVCSQEAKKSAYCPYSH |
| | FPVGAALLTQEGRIFKGCNIENACYPLGICAERTAIQKAVSEGYKDFRAIAIASDMQ |
| | DDFISPCGACRQVMREFGTNWPVYMTKPDGTYIVMTVQELLPSSFGPEDLQKTQ |
| | (SEQ ID NO: 302) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)7 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSGGGGSMAQKRPACTLKPECVQQLLVCSQEAKKSAY |
| | CPYSHFPVGAALLTQEGRIFKGCNIENACYPLGICAERTAIQKAVSEGYKDFRAIAI |
| | ASDMQDDFISPCGACRQVMREFGTNWPVYMTKPDGTYIVMTVQELLPSSFGPEDL |
| | QKTQ (SEQ ID NO: 303) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)8 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSGGGGSGGGGSMAQKRPACTLKPECVQQLLVCSQEA |
| | KKSAYCPYSHFPVGAALLTQEGRIFKGCNIENACYPLGICAERTAIQKAVSEGYKDF |
| | RAIAIASDMQDDFISPCGACRQVMREFGTNWPVYMTKPDGTYIVMTVQELLPSSFG |
| | PEDLQKTQ (SEQ ID NO: 304) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)9 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQ (SEQ ID NO: 305) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)10 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQ (SEQ ID NO: 306) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)11 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSGGGGSGGGGSMAQKRPACT |
| | LKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQ (SEQ ID NO: 307) |
| Ab66_HC_ | EVQLVESGGGLVQPGGSLRLSCAASGETFSDHYMDWVRQAPGKGLASGETFSDH |
| (G4S)3_LC_ | YMDWVRQAPGKGLEWVGRIRNKASSYTTEYAASVKGRFTISRDDSKNSLYLQMN |
| (G4S)112 | SLKTEDTAVYYCAREPKYWIDFDLWGRGTLVTVSSGGGGSGGGGSGGGGSDIQM |
| linker_CDA | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIKGGGGSGGGGSG |
| | GGGSGGGGSGGGGSGGGGSGGGGSGGGGSGGGGSGGGGSGGGGSGGGGSMAQK |
| | RPACTLKPECVQQLLVCSQEAKKSAYCPYSHFPVGAALLTQEGRIFKGCNIENACY |
| | PLGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 308) |
| Ab68_HC_ | EVQLVESGGGLVQPGRSLRLSCTASGFTFSDHDMNWVRQAPGKGLEWVGRTRNA |
| (G4S)3_LC_G4S | AGSYTTEYAASVKGRFTISRDDSKNSLYLQMNSLKTEDTAVYYCAREPKYWIDFD |
| linker_CDA | LWGRGTLVTSSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIKGGGGSMAQKRPACTLKPECVQQLLVCSQEAKKSAYC |
| | PYSHFPVGAALLTQEGRIFKGCNIENACYPLGICAERTAIQKAVSEGYKDFRAIAIAS |
| | DMQDDFISPCGACRQVMREFGTNWPVYMTKPDGTYIVMTVQELLPSSFGPEDLQK |
| | TQ (SEQ ID NO: 309) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)3 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSMAQKRPACTLKPECVQQLLVC |
| | SQEAKKSAYCPYSHFPVGAALLTQEGRIFKGCNIENACYPLGICAERTAIQKAVSEG |
| | YKDFRAIAIASDMQDDFISPCGACRQVMREFGTNWPVYMTKPDGTYIVMTVQELL |
| | PSSFGPEDLQKTQ (SEQ ID NO: 310) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)4 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSMAQKRPACTLKPECVQ |
| | QLLVCSQEAKKSAYCPYSHFPVGAALLTQEGRIFKGCNIENACYPLGICAERTAIQK |
| | AVSEGYKDFRAIAIASDMQDDFISPCGACRQVMREFGTNWPVYMTKPDGTYIVMT |
| | VQELLPSSFGPEDLQKTQ (SEQ ID NO: 311) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)5 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSMAQKRPACTL |
| | KPECVQQLLVCSQEAKKSAYCPYSHFPVGAALLTQEGRIFKGCNIENACYPLGICA |
| | ERTAIQKAVSEGYKDFRAIAIASDMQDDFISPCGACRQVMREFGTNWPVYMTKPD |
| | GTYIVMTVQELLPSSFGPEDLQKTQ (SEQ ID NO: 312) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)6 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSMAQKR |
| | PACTLKPECVQQLLVCSQEAKKSAYCPYSHFPVGAALLTQEGRIFKGCNIENACYP |
| | LGICAERTAIQKAVSEGYKDFRAIAIASDMQDDFISPCGACRQVMREFGTNWPVY |
| | MTKPDGTYIVMTVQELLPSSFGPEDLQKTQ (SEQ ID NO: 313) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)7 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSGGGGS |
| | MAQKRPACTLKPECVQQLLVCSQEAKKSAYCPYSHFPVGAALLTQEGRIFKGCNIE |
| | NACYPLGICAERTAIQKAVSEGYKDFRAIAIASDMQDDFISPCGACRQVMREFGTN |
| | WPVYMTKPDGTYIVMTVQELLPSSFGPEDLQKTQ (SEQ ID NO: 314) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)8 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSGGGGS |
| | GGGGSMAQKRPACTLKPECVQQLLVCSQEAKKSAYCPYSHFPVGAALLTQEGRIF |
| | KGCNIENACYPLGICAERTAIQKAVSEGYKDFRAIAIASDMQDDFISPCGACRQVM |
| | REFGTNWPVYMTKPDGTYIVMTVQELLPSSFGPEDLQKTQ (SEQ ID NO: 315) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)9 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSGGGGS |
| | GGGGSGGGGSMAQKRPACTLKPECVQQLLVCSQEAKKSAYCPYSHFPVGAALLT |
| | QEGRIFKGCNIENACYPLGICAERTAIQKAVSEGYKDFRAIAIASDMQDDFISPCGA |
| | CRQVMREFGTNWPVYMTKPDGTYIVMTVQELLPSSFGPEDLQKTQ (SEQ ID NO: |
| | 316) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)10 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSGGGGS |
| | GGGGSGGGGSGGGGSMAQKRPACTLKPECVQQLLVCSQEAKKSAYCPYSHFPVG |
| | AALLTQEGRIFKGCNIENACYPLGICAERTAIQKAVSEGYKDFRAIAIASDMQDDFI |
| | SPCGACRQVMREFGTNWPVYMTKPDGTYIVMTVQELLPSSFGPEDLQKTQ (SEQ |
| | ID NO: 317) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)11 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSGGGGS |
| | GGGGSGGGGSGGGGSGGGGSMAQKRPACTLKPECVQQLLVCSQEAKKSAYCPYS |
| | HFPVGAALLTQEGRIFKGCNIENACYPLGICAERTAIQKAVSEGYKDFRAIAIASDM |
| | QDDFISPCGACRQVMREFGTNWPVYMTKPDGTYIVMTVQELLPSSFGPEDLQKTQ |
| | (SEQ ID NO: 318) |
| Ab68_HC_ | EVQLVESGGGLVQPGRSLRLSCTASGFTFSDHDMNWVRQAPGKGLEWVGRTRNA |
| (G4S)3_LC_ | AGSYTTEYAASVKGRFTISRDDSKNSLYLQMNSLKTEDTAVYYCAREPKYWIDFD |
| (G4S)112 | LWGRGTLVTSSGGGGSGGGGSGGGGSDIQMTQSPSSLSASVGDRVTITCRASQSISS |
| linker_CDA | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSGGGGSGGGGSGGGGSGGGGS |
| | GGGGSGGGGSGGGGSGGGGSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQ (SEQ ID NO: 319) |
| Ab69_HC_ | DIQMTQSPSSLSASVGDRVTITCRASQSISSYLNWYQQKPGKAPKLLIYAASSLQSG |
| (G4S)3_LC_G4S | VPSRFSGSGSGTDFTLTISSLQPEDFATYYCQQSYIAPYIFGGGTKVEIKGGGGSGGG |
| linker_CDA | GSGGGGSEVQLVESGGGLVQPGGSLRLSCAASGETFVDHDMDWVRQAPGKGLE |
| | WVGRTRNKLGSYTTEYAASVKGRETGRDDSKNSLYLQMNSLKTEDTAVYYCARE |
| | PKYWIDFDLWGRGTLVTVSSGGGGSMAQKRPACTLKPECVQQLLVCSQEAKKSA |
| | YCPYSHFPVGAALLTQEGRIFKGCNIENACYPLGICAERTAIQKAVSEGYKDFRAIA |
| | IASDMQDDFISPCGACRQVMREFGTNWPVYMTKPDGTYIVMTVQELLPSSFGPED |
| | LQKTQ (SEQ ID NO: 320) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)3 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSMAQKRPACTLKPECVQQLL |
| | VCSQEAKKSAYCPYSHFPVGAALLTQEGRIFKGCNIENACYPLGICAERTAIQKAVS |
| | EGYKDFRAIAIASDMQDDFISPCGACRQVMREFGTNWPVYMTKPDGTYIVMTVQE |
| | LLPSSFGPEDLQKTQ (SEQ ID NO: 321) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)4 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSMAQKRPACTLKPEC |
| | VQQLLVCSQEAKKSAYCPYSHFPVGAALLTQEGRIFKGCNIENACYPLGICAERTAI |
| | QKAVSEGYKDFRAIAIASDMQDDFISPCGACRQVMREFGTNWPVYMTKPDGTYIV |
| | MTVQELLPSSFGPEDLQKTQ (SEQ ID NO: 322) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)5 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSMAQKRPAC |
| | TLKPECVQQLLVCSQEAKKSAYCPYSHFPVGAALLTQEGRIFKGCNIENACYPLGIC |
| | AERTAIQKAVSEGYKDFRAIAIASDMQDDFISPCGACRQVMREFGTNWPVYMTKP |
| | DGTYIVMTVQELLPSSFGPEDLQKTQ (SEQ ID NO: 323) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)6 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSMAQ |
| | KRPACTLKPECVQQLLVCSQEAKKSAYCPYSHFPVGAALLTQEGRIFKGCNIENAC |
| | YPLGICAERTAIQKAVSEGYKDFRAIAIASDMQDDFISPCGACRQVMREFGTNWPV |
| | YMTKPDGTYIVMTVQELLPSSFGPEDLQKTQ (SEQ ID NO: 324) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)7 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSGGG |
| | GSMAQKRPACTLKPECVQQLLVCSQEAKKSAYCPYSHFPVGAALLTQEGRIFKGC |
| | NIENACYPLGICAERTAIQKAVSEGYKDFRAIAIASDMQDDFISPCGACRQVMREFG |
| | TNWPVYMTKPDGTYIVMTVQELLPSSFGPEDLQKTQ (SEQ ID NO: 325) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)8 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSGGG |
| | GSGGGGSMAQKRPACTLKPECVQQLLVCSQEAKKSAYCPYSHFPVGAALLTQEGR |
| | IFKGCNIENACYPLGICAERTAIQKAVSEGYKDFRAIAIASDMQDDFISPCGACRQV |
| | MREFGTNWPVYMTKPDGTYIVMTVQELLPSSFGPEDLQKTQ (SEQ ID NO: 326) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)9 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSGGG |
| | GSGGGGSGGGGSMAQKRPACTLKPECVQQLLVCSQEAKKSAYCPYSHFPVGAAL |
| | LTQEGRIFKGCNIENACYPLGICAERTAIQKAVSEGYKDFRAIAIASDMQDDFISPCG |
| | ACRQVMREFGTNWPVYMTKPDGTYIVMTVQELLPSSFGPEDLQKTQ (SEQ ID NO: |
| | 327) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)10 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSGGG |
| | GSGGGGSGGGGSGGGGSMAQKRPACTLKPECVQQLLVCSQEAKKSAYCPYSHFP |
| | VGAALLTQEGRIFKGCNIENACYPLGICAERTAIQKAVSEGYKDFRAIAIASDMQD |
| | DFISPCGACRQVMREFGTNWPVYMTKPDGTYIVMTVQELLPSSFGPEDLQKTQ |
| | (SEQ ID NO: 328) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)11 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSGGG |
| | GSGGGGSGGGGSGGGGSGGGGSMAQKRPACTLKPECVQQLLVCSQEAKKSAYCP |
| | YSHFPVGAALLTQEGRIFKGCNIENACYPLGICAERTAIQKAVSEGYKDFRAIAIAS |
| | DMQDDFISPCGACRQVMREFGTNWPVYMTKPDGTYIVMTVQELLPSSFGPEDLQK |
| | TQ (SEQ ID NO: 329) |
| Ab69_HC_ | DIQMTQSPSSLSASVGDRVTITCRASQSISSYLNWYQQKPGKAPKLLIYAASSLQSG |
| (G4S)3_LC_ | VPSRFSGSGSGTDFTLTISSLQPEDFATYYCQQSYIAPYIFGGGTKVEIKGGGGSGGG |
| (G4S)112 | GSGGGGSEVQLVESGGGLVQPGGSLRLSCAASGETFVDHDMDWVRQAPGKGLE |
| linker_CDA | WVGRTRNKLGSYTTEYAASVKGRETGRDDSKNSLYLQMNSLKTEDTAVYYCARE |
| | PKYWIDFDLWGRGTLVTVSSGGGGSGGGGSGGGGSGGGGSGGGGSGGGGSGGG |
| | GSGGGGSGGGGSGGGGSGGGGSGGGGSMAQKRPACTLKPECVQQLLVCSQEAKK |
| | SAYCPYSHFPVGAALLTQEGRIFKGCNIENACYPLGICAERTAIQKAVSEGYKDFRA |
| | IAIASDMQDDFISPCGACRQVMREFGTNWPVYMTKPDGTYIVMTVQELLPSSFGPE |
| | DLQKTQ (SEQ ID NO: 330) |
| CDA_G4S | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSVSASVG |
| HC | DRVTITCRASQGISSWLAWYQQKPGKAPKLLLIYAASSLQSGVPSRFSGSGSGTDFT |
| | LTISSLQPEDFATYYCQQTNSFPYTFGGGTKVEIKGGGGSGGGGSGGGGSEVQLLE |
| | SGGGLVQPGGSLRLSCAASGETFSNYAMSWVRQAPGKGLEWVSAISGSGGSTYYA |
| | DSVKGRFTISRDNSKNTLYLQMNSLRAEDTAVYYCAKGPPTYHTNYYYMDVWGK |
| | GTTVTVSS (SEQ ID NO: 331) |
| CDA_G4S | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSVSASVG |
| HC | DRVTITCRASQGISSWLAWYQQKPGKAPKLLIYAASSLQQSGVPSRFSGSGSGTDF |
| | TLTISSLQPEDFATYYCQQTNSEPYTFGGGTKVEIKGGGGSGGGGSGGGGSEVQLL |
| | ESGGGLVQPGGSLRLSCAASGETESSYVMIWVRQAPGKGLEWVSSISGDSVTTYY |
| | ADSVKGRFTISRDNSKNTLYLQMNSLRAEDTAVYYCAKGPPTYHTNYYYMDVWG |
| | KGTTVTVSS (SEQ ID NO: 332) |
| CDA_G4S | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSLSASVG |
| HC | DRVTITCRASQSISSYLNWYQQKPGKAPKLLIYAASSLQSGVPSRFSGSGSGTDFTL |
| | TISSLQPEDFATYYCQQSYIAPYTFGGGTKVEIKGGGGSGGGGSGGGGSEVQLVES |
| | GGGLVQPGGSLRLSCAASGETFSDHYMDWVRQAPGKGLASGETFSDHYMDWVR |
| | QAPGKGLEWVGRIRNKASSYTTEYAASVKGRFTISRDDSKNSLYLQMNSLKTEDT |
| | AVYYCAREPKYWIDFDLWGRGTLVTVSS (SEQ ID NO: 333) |
| CDA_G4S | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSDIQMTQSPSSLSASVG |
| HC | DRVTITCRASQSISSYLNWYQQKPGKAPKLLIYAASSLQSGVPSRFSGSGSGTDFTL |
| | TISSLQPEDFATYYCQQSYIAPYTFGGGTKVEIKGGGGSGGGGSGGGGSEVQLVES |
| | GGGLVQPGRSLRLSCTASGFTFSDHDMNWVRQAPGKGLEWVGRTRNAAGSYTTE |
| | YAASVKGRFTISRDDSKNSLYLQMNSLKTEDTAVYYCAREPKYWIDFDLWGRGTL |
| | VTSS (SEQ ID NO: 334) |
| CDA_G4S | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSEVQLVESGGGLVQPGG |
| HC | SLRLSCAASGETFVDHDMDWVRQAPGKGLEWVGRTRNKLGSYTTEYAASVKGRE |
| | TGRDDSKNSLYLQMNSLKTEDTAVYYCAREPKYWIDFDLWGRGTLVTVSSGGGG |
| | SGGGGSGGGGSDIQMTQSPSSLSASVGDRVTITCRASQSISSYLNWYQQKPGKAPK |
| | LLIYAASSLQSGVPSRFSGSGSGTDFTLTISSLQPEDFATYYCQQSYIAPYIFGGGTK |
| | VEIK (SEQ ID NO: 335) |
| CDA_(G4S)3 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC | QSPSSVSASVGDRVTITCRASQGISSWLAWYQQKPGKAPKLLLIYAASSLQSGVPSR |
| | FSGSGSGTDFTLTISSLQPEDFATYYCQQTNSFPYTFGGGTKVEIKGGGGSGGGGSG |
| | GGGSEVQLLESGGGLVQPGGSLRLSCAASGETFSNYAMSWVRQAPGKGLEWVSAI |
| | SGSGGSTYYADSVKGRFTISRDNSKNTLYLQMNSLRAEDTAVYYCAKGPPTYHTN |
| | YYYMDVWGKGTTVTVSS (SEQ ID NO: 336) |
| CDA_(G4S)3 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC | QSPSSVSASVGDRVTITCRASQGISSWLAWYQQKPGKAPKLLIYAASSLQQSGVPS |
| | RFSGSGSGTDFTLTISSLQPEDFATYYCQQTNSEPYTFGGGTKVEIKGGGGSGGGGS |
| | GGGGSEVQLLESGGGLVQPGGSLRLSCAASGETESSYVMIWVRQAPGKGLEWVSS |
| | ISGDSVTTYYADSVKGRFTISRDNSKNTLYLQMNSLRAEDTAVYYCAKGPPTYHT |
| | NYYYMDVWGKGTTVTVSS (SEQ ID NO: 337) |
| CDA_(G4S)3 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC | QSPSSLSASVGDRVTITCRASQSISSYLNWYQQKPGKAPKLLIYAASSLQSGVPSRFS |
| | GSGSGTDFTLTISSLQPEDFATYYCQQSYIAPYTFGGGTKVEIKGGGGSGGGGSGG |
| | GGSEVQLVESGGGLVQPGGSLRLSCAASGETFSDHYMDWVRQAPGKGLASGETFS |
| | DHYMDWVRQAPGKGLEWVGRIRNKASSYTTEYAASVKGRFTISRDDSKNSLYLQ |
| | MNSLKTEDTAVYYCAREPKYWIDFDLWGRGTLVTVSS (SEQ ID NO: 338) |
| CDA_(G4S)3 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSDIQMT |
| HC | QSPSSLSASVGDRVTITCRASQSISSYLNWYQQKPGKAPKLLIYAASSLQSGVPSRFS |
| | GSGSGTDFTLTISSLQPEDFATYYCQQSYIAPYTFGGGTKVEIKGGGGSGGGGSGG |
| | GGSEVQLVESGGGLVQPGRSLRLSCTASGFTFSDHDMNWVRQAPGKGLEWVGRT |
| | RNAAGSYTTEYAASVKGRFTISRDDSKNSLYLQMNSLKTEDTAVYYCAREPKYWI |
| | DFDLWGRGTLVTSS (SEQ ID NO: 339) |
| CDA_(G4S)3 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSEVQLV |
| HC | ESGGGLVQPGGSLRLSCAASGETFVDHDMDWVRQAPGKGLEWVGRTRNKLGSYT |
| | TEYAASVKGRETGRDDSKNSLYLQMNSLKTEDTAVYYCAREPKYWIDFDLWGRG |
| | TLVTVSSGGGGGGGGSGGGGSDIQMTQSPSSLSASVGDRVTITCRASQSISSYLN |
| | WYQQKPGKAPKLLIYAASSLQSGVPSRFSGSGSGTDFTLTISSLQPEDFATYYCQQS |
| | YIAPYIFGGGTKVEIK (SEQ ID NO: 340) |
| CDA_(G4S)4 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | DIQMTQSPSSVSASVGDRVTITCRASQGISSWLAWYQQKPGKAPKLLLIYAASSLQ |
| | SGVPSRFSGSGSGTDFTLTISSLQPEDFATYYCQQTNSFPYTFGGGTKVEIKGGGGS |
| | GGGGSGGGGSEVQLLESGGGLVQPGGSLRLSCAASGETFSNYAMSWVRQAPGKG |
| | LEWVSAISGSGGSTYYADSVKGRFTISRDNSKNTLYLQMNSLRAEDTAVYYCAKG |
| | PPTYHTNYYYMDVWGKGTTVTVSS (SEQ ID NO: 341) |
| CDA_(G4S)4 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | DIQMTQSPSSVSASVGDRVTITCRASQGISSWLAWYQQKPGKAPKLLIYAASSLQQ |
| | SGVPSRFSGSGSGTDFTLTISSLQPEDFATYYCQQTNSEPYTFGGGTKVEIKGGGGS |
| | GGGGSGGGGSEVQLLESGGGLVQPGGSLRLSCAASGETESSYVMIWVRQAPGKGL |
| | EWVSSISGDSVTTYYADSVKGRFTISRDNSKNTLYLQMNSLRAEDTAVYYCAKGP |
| | PTYHTNYYYMDVWGKGTTVTVSS (SEQ ID NO: 342) |
| CDA_(G4S)4 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYTFGGGTKVEIKGGGGSGG |
| | GGSGGGGSEVQLVESGGGLVQPGGSLRLSCAASGETFSDHYMDWVRQAPGKGLA |
| | SGETFSDHYMDWVRQAPGKGLEWVGRIRNKASSYTTEYAASVKGRFTISRDDSKN |
| | SLYLQMNSLKTEDTAVYYCAREPKYWIDFDLWGRGTLVTVSS (SEQ ID NO: 343) |
| CDA_(G4S)4 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYTFGGGTKVEIKGGGGSGG |
| | GGSGGGGSEVQLVESGGGLVQPGRSLRLSCTASGFTFSDHDMNWVRQAPGKGLE |
| | WVGRTRNAAGSYTTEYAASVKGRFTISRDDSKNSLYLQMNSLKTEDTAVYYCAR |
| | EPKYWIDFDLWGRGTLVTSS (SEQ ID NO: 344) |
| CDA_(G4S)4 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | EVQLVESGGGLVQPGGSLRLSCAASGETFVDHDMDWVRQAPGKGLEWVGRTRN |
| | KLGSYTTEYAASVKGRETGRDDSKNSLYLQMNSLKTEDTAVYYCAREPKYWIDF |
| | DLWGRGTLVTVSSGGGGSGGGGSGGGGSDIQMTQSPSSLSASVGDRVTITCRASQS |
| | ISSYLNWYQQKPGKAPKLLIYAASSLQSGVPSRFSGSGSGTDFTLTISSLQPEDFATY |
| | YCQQSYIAPYIFGGGTKVEIK (SEQ ID NO: 345) |
| CDA_(G4S)5 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSDIQMTQSPSSVSASVGDRVTITCRASQGISSWLAWYQQKPGKAPKLLLIYA |
| | ASSLQSGVPSRFSGSGSGTDFTLTISSLQPEDFATYYCQQTNSFPYTFGGGTKVEIKG |
| | GGGSGGGGSGGGGSEVQLLESGGGLVQPGGSLRLSCAASGETFSNYAMSWVRQA |
| | PGKGLEWVSAISGSGGSTYYADSVKGRFTISRDNSKNTLYLQMNSLRAEDTAVYY |
| | CAKGPPTYHTNYYYMDVWGKGTTVTVSS (SEQ ID NO: 346) |
| CDA_(G4S)5 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSDIQMTQSPSSVSASVGDRVTITCRASQGISSWLAWYQQKPGKAPKLLIYAA |
| | SSLQQSGVPSRFSGSGSGTDFTLTISSLQPEDFATYYCQQTNSEPYTFGGGTKVEIKG |
| | GGGSGGGGSGGGGSEVQLLESGGGLVQPGGSLRLSCAASGETESSYVMIWVRQAP |
| | GKGLEWVSSISGDSVTTYYADSVKGRFTISRDNSKNTLYLQMNSLRAEDTAVYYC |
| | AKGPPTYHTNYYYMDVWGKGTTVTVSS (SEQ ID NO: 347) |
| CDA_(G4S)5 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYTFGGGTKVEIKGGG |
| | GSGGGGSGGGGSEVQLVESGGGLVQPGGSLRLSCAASGETFSDHYMDWVRQAPG |
| | KGLASGETFSDHYMDWVRQAPGKGLEWVGRIRNKASSYTTEYAASVKGRFTISRD |
| | DSKNSLYLQMNSLKTEDTAVYYCAREPKYWIDFDLWGRGTLVTVSS (SEQ ID NO: |
| | 348) |
| CDA_(G4S)5 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYTFGGGTKVEIKGGG |
| | GSGGGGSGGGGSEVQLVESGGGLVQPGRSLRLSCTASGFTFSDHDMNWVRQAPG |
| | KGLEWVGRTRNAAGSYTTEYAASVKGRFTISRDDSKNSLYLQMNSLKTEDTAVY |
| | YCAREPKYWIDFDLWGRGTLVTSS (SEQ ID NO: 349) |
| CDA_(G4S)5 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSEVQLVESGGGLVQPGGSLRLSCAASGETFVDHDMDWVRQAPGKGLEWV |
| | GRTRNKLGSYTTEYAASVKGRETGRDDSKNSLYLQMNSLKTEDTAVYYCAREPK |
| | YWIDFDLWGRGTLVTVSSGGGGSGGGGSGGGGSDIQMTQSPSSLSASVGDRVTIT |
| | CRASQSISSYLNWYQQKPGKAPKLLIYAASSLQSGVPSRFSGSGSGTDFTLTISSLQP |
| | EDFATYYCQQSYIAPYIFGGGTKVEIK (SEQ ID NO: 350) |
| CDA_(G4S)6 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSDIQMTQSPSSVSASVGDRVTITCRASQGISSWLAWYQQKPGKAPK |
| | LLLIYAASSLQSGVPSRFSGSGSGTDFTLTISSLQPEDFATYYCQQTNSFPYTFGGGT |
| | KVEIKGGGGSGGGGSGGGGSEVQLLESGGGLVQPGGSLRLSCAASGETFSNYAMS |
| | WVRQAPGKGLEWVSAISGSGGSTYYADSVKGRFTISRDNSKNTLYLQMNSLRAED |
| | TAVYYCAKGPPTYHTNYYYMDVWGKGTTVTVSS (SEQ ID NO: 351) |
| CDA_(G4S)6 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSDIQMTQSPSSVSASVGDRVTITCRASQGISSWLAWYQQKPGKAPK |
| | LLIYAASSLQQSGVPSRFSGSGSGTDFTLTISSLQPEDFATYYCQQTNSEPYTFGGGT |
| | KVEIKGGGGSGGGGGGGGSEVQLLESGGGLVQPGGSLRLSCAASGETESSYVMI |
| | WVRQAPGKGLEWVSSISGDSVTTYYADSVKGRFTISRDNSKNTLYLQMNSLRAED |
| | TAVYYCAKGPPTYHTNYYYMDVWGKGTTVTVSS (SEQ ID NO: 352) |
| CDA_(G4S)6 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIKGGGGSGGGGSGGGGSEVQLVESGGGLVQPGGSLRLSCAASGETFSDHYMDW |
| | VRQAPGKGLASGETFSDHYMDWVRQAPGKGLEWVGRIRNKASSYTTEYAASVKG |
| | RFTISRDDSKNSLYLQMNSLKTEDTAVYYCAREPKYWIDFDLWGRGTLVTVSS |
| | (SEQ ID NO: 353) |
| CDA_(G4S)6 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIKGGGGSGGGGGGGGSEVQLVESGGGLVQPGRSLRLSCTASGFTFSDHDMNWV |
| | RQAPGKGLEWVGRTRNAAGSYTTEYAASVKGRFTISRDDSKNSLYLQMNSLKTED |
| | TAVYYCAREPKYWIDFDLWGRGTLVTSS (SEQ ID NO: 354) |
| CDA_(G4S)6 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGGGGGSEVQLVESGGGLVQPGGSLRLSCAASGETFVDHDMDWVRQAPGK |
| | GLEWVGRTRNKLGSYTTEYAASVKGRETGRDDSKNSLYLQMNSLKTEDTAVYYC |
| | AREPKYWIDFDLWGRGTLVTVSSGGGGSGGGGSGGGGSDIQMTQSPSSLSASVGD |
| | RVTITCRASQSISSYLNWYQQKPGKAPKLLIYAASSLQSGVPSRFSGSGSGTDFTLTI |
| | SSLQPEDFATYYCQQSYIAPYIFGGGTKVEIK (SEQ ID NO: 355) |
| CDA_(G4S)7 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSDIQMTQSPSSVSASVGDRVTITCRASQGISSWLAWYQQKP |
| | GKAPKLLLIYAASSLQSGVPSRFSGSGSGTDFTLTISSLQPEDFATYYCQQTNSFPYT |
| | FGGGTKVEIKGGGGSGGGGSGGGGSEVQLLESGGGLVQPGGSLRLSCAASGETFS |
| | NYAMSWVRQAPGKGLEWVSAISGSGGSTYYADSVKGRFTISRDNSKNTLYLQMN |
| | SLRAEDTAVYYCAKGPPTYHTNYYYMDVWGKGTTVTVSS (SEQ ID NO: 356) |
| CDA_(G4S)7 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSDIQMTQSPSSVSASVGDRVTITCRASQGISSWLAWYQQKP |
| | GKAPKLLIYAASSLQQSGVPSRFSGSGSGTDFTLTISSLQPEDFATYYCQQTNSEPYT |
| | FGGGTKVEIKGGGGSGGGGSGGGGSEVQLLESGGGLVQPGGSLRLSCAASGETESS |
| | YVMIWVRQAPGKGLEWVSSISGDSVTTYYADSVKGRFTISRDNSKNTLYLQMNSL |
| | RAEDTAVYYCAKGPPTYHTNYYYMDVWGKGTTVTVSS (SEQ ID NO: 357) |
| CDA_(G4S)7 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSDIQMTQSPSSLSASVGDRVTITCRASQSISSYLNWYQQKPG |
| | KAPKLLIYAASSLQSGVPSRFSGSGSGTDFTLTISSLQPEDFATYYCQQSYIAPYTFG |
| | GGTKVEIKGGGGSGGGGSGGGGSEVQLVESGGGLVQPGGSLRLSCAASGETFSDH |
| | YMDWVRQAPGKGLASGETFSDHYMDWVRQAPGKGLEWVGRIRNKASSYTTEYA |
| | ASVKGRFTISRDDSKNSLYLQMNSLKTEDTAVYYCAREPKYWIDFDLWGRGTLVT |
| | VSS (SEQ ID NO: 358) |
| CDA_(G4S)7 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSDIQMTQSPSSLSASVGDRVTITCRASQSISSYLNWYQQKPG |
| | KAPKLLIYAASSLQSGVPSRFSGSGSGTDFTLTISSLQPEDFATYYCQQSYIAPYTFG |
| | GGTKVEIKGGGGSGGGGSGGGGSEVQLVESGGGLVQPGRSLRLSCTASGFTFSDH |
| | DMNWVRQAPGKGLEWVGRTRNAAGSYTTEYAASVKGRFTISRDDSKNSLYLQM |
| | NSLKTEDTAVYYCAREPKYWIDFDLWGRGTLVTSS (SEQ ID NO: 359) |
| CDA_(G4S)7 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSEVQLVESGGGLVQPGGSLRLSCAASGETFVDHDMDWVR |
| | QAPGKGLEWVGRTRNKLGSYTTEYAASVKGRETGRDDSKNSLYLQMNSLKTEDT |
| | AVYYCAREPKYWIDFDLWGRGTLVTVSSGGGGSGGGGSGGGGSDIQMTQSPSSLS |
| | ASVGDRVTITCRASQSISSYLNWYQQKPGKAPKLLIYAASSLQSGVPSRFSGSGSGT |
| | DFTLTISSLQPEDFATYYCQQSYIAPYIFGGGTKVEIK (SEQ ID NO: 360) |
| CDA_(G4S)8 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSDIQMTQSPSSVSASVGDRVTITCRASQGISSWLAW |
| | YQQKPGKAPKLLLIYAASSLQSGVPSRFSGSGSGTDFTLTISSLQPEDFATYYCQQT |
| | NSFPYTFGGGTKVEIKGGGGSGGGGSGGGGSEVQLLESGGGLVQPGGSLRLSCAA |
| | SGETFSNYAMSWVRQAPGKGLEWVSAISGSGGSTYYADSVKGRFTISRDNSKNTL |
| | YLQMNSLRAEDTAVYYCAKGPPTYHTNYYYMDVWGKGTTVTVSS (SEQ ID NO: |
| | 361) |
| CDA_(G4S)8 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSDIQMTQSPSSVSASVGDRVTITCRASQGISSWLAW |
| | YQQKPGKAPKLLIYAASSLQQSGVPSRFSGSGSGTDFTLTISSLQPEDFATYYCQQT |
| | NSEPYTFGGGTKVEIKGGGGSGGGGSGGGGSEVQLLESGGGLVQPGGSLRLSCAA |
| | SGETESSYVMIWVRQAPGKGLEWVSSISGDSVTTYYADSVKGRFTISRDNSKNTLY |
| | LQMNSLRAEDTAVYYCAKGPPTYHTNYYYMDVWGKGTTVTVSS (SEQ ID NO: |
| | 362) |
| CDA_(G4S)8 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYTFGGGTKVEIKGGGGSGGGGSGGGGSEVQLVESGGGLVQPGGSLRLSCAASGE |
| | TFSDHYMDWVRQAPGKGLASGETFSDHYMDWVRQAPGKGLEWVGRIRNKASSY |
| | TTEYAASVKGRFTISRDDSKNSLYLQMNSLKTEDTAVYYCAREPKYWIDFDLWGR |
| | GTLVTVSS (SEQ ID NO: 363) |
| CDA_(G4S)8 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYTFGGGTKVEIKGGGGSGGGGSGGGGSEVQLVESGGGLVQPGRSLRLSCTASGF |
| | TFSDHDMNWVRQAPGKGLEWVGRTRNAAGSYTTEYAASVKGRFTISRDDSKNSL |
| | YLQMNSLKTEDTAVYYCAREPKYWIDFDLWGRGTLVTSS (SEQ ID NO: 364) |
| CDA_(G4S)8 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSEVQLVESGGGLVQPGGSLRLSCAASGETFVDHDM |
| | DWVRQAPGKGLEWVGRTRNKLGSYTTEYAASVKGRETGRDDSKNSLYLQMNSL |
| | KTEDTAVYYCAREPKYWIDFDLWGRGTLVTVSSGGGGSGGGGSGGGGSDIQMTQ |
| | SPSSLSASVGDRVTITCRASQSISSYLNWYQQKPGKAPKLLIYAASSLQSGVPSRFSG |
| | SGSGTDFTLTISSLQPEDFATYYCQQSYIAPYIFGGGTKVEIK (SEQ ID NO: 365) |
| CDA_(G4S)9 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSDIQMTQSPSSVSASVGDRVTITCRASQGISS |
| | WLAWYQQKPGKAPKLLLIYAASSLQSGVPSRFSGSGSGTDFTLTISSLQPEDFATYY |
| | CQQTNSFPYTFGGGTKVEIKGGGGSGGGGSGGGGSEVQLLESGGGLVQPGGSLRL |
| | SCAASGETFSNYAMSWVRQAPGKGLEWVSAISGSGGSTYYADSVKGRFTISRDNS |
| | KNTLYLQMNSLRAEDTAVYYCAKGPPTYHTNYYYMDVWGKGTTVTVSS (SEQ ID |
| | NO: 366) |
| CDA_(G4S)9 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSDIQMTQSPSSVSASVGDRVTITCRASQGISS |
| | WLAWYQQKPGKAPKLLIYAASSLQQSGVPSRFSGSGSGTDFTLTISSLQPEDFATY |
| | YCQQTNSEPYTFGGGTKVEIKGGGGSGGGGSGGGGSEVQLLESGGGLVQPGGSLR |
| | LSCAASGETESSYVMIWVRQAPGKGLEWVSSISGDSVTTYYADSVKGRFTISRDNS |
| | KNTLYLQMNSLRAEDTAVYYCAKGPPTYHTNYYYMDVWGKGTTVTVSS (SEQ ID |
| | NO: 367) |
| CDA_(G4S)9 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSEVQLVESGGGLVQPGGSLRLS |
| | CAASGETFSDHYMDWVRQAPGKGLASGETFSDHYMDWVRQAPGKGLEWVGRIR |
| | NKASSYTTEYAASVKGRFTISRDDSKNSLYLQMNSLKTEDTAVYYCAREPKYWID |
| | FDLWGRGTLVTVSS (SEQ ID NO: 368) |
| CDA_(G4S)9 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIKGGGGSGGGGSGGGGSEVQLVESGGGLVQPGRSLRLS |
| | CTASGFTFSDHDMNWVRQAPGKGLEWVGRTRNAAGSYTTEYAASVKGRFTISRD |
| | DSKNSLYLQMNSLKTEDTAVYYCAREPKYWIDFDLWGRGTLVTSS (SEQ ID NO: |
| | 369) |
| CDA_(G4S)9 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSEVQLVESGGGLVQPGGSLRLSCAASGETF |
| | VDHDMDWVRQAPGKGLEWVGRTRNKLGSYTTEYAASVKGRETGRDDSKNSLYL |
| | QMNSLKTEDTAVYYCAREPKYWIDFDLWGRGTLVTVSSGGGGSGGGGSGGGGSD |
| | IQMTQSPSSLSASVGDRVTITCRASQSISSYLNWYQQKPGKAPKLLIYAASSLQSGV |
| | PSRFSGSGSGTDFTLTISSLQPEDFATYYCQQSYIAPYIFGGGTKVEIK (SEQ ID NO: |
| | 370) |
| CDA_(G4S)10 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSDIQMTQSPSSVSASVGDRVTITCRA |
| | SQGISSWLAWYQQKPGKAPKLLLIYAASSLQSGVPSRFSGSGSGTDFTLTISSLQPE |
| | DFATYYCQQTNSFPYTFGGGTKVEIKGGGGSGGGGSGGGGSEVQLLESGGGLVQP |
| | GGSLRLSCAASGETFSNYAMSWVRQAPGKGLEWVSAISGSGGSTYYADSVKGRFT |
| | ISRDNSKNTLYLQMNSLRAEDTAVYYCAKGPPTYHTNYYYMDVWGKGTTVTVSS |
| | (SEQ ID NO: 371) |
| CDA_(G4S)10 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSDIQMTQSPSSVSASVGDRVTITCRA |
| | SQGISSWLAWYQQKPGKAPKLLIYAASSLQQSGVPSRFSGSGSGTDFTLTISSLQPE |
| | DFATYYCQQTNSEPYTFGGGTKVEIKGGGGSGGGGSGGGGSEVQLLESGGGLVQP |
| | GGSLRLSCAASGETESSYVMIWVRQAPGKGLEWVSSISGDSVTTYYADSVKGRFTI |
| | SRDNSKNTLYLQMNSLRAEDTAVYYCAKGPPTYHTNYYYMDVWGKGTTVTVSS |
| | (SEQ ID NO: 372) |
| CDA_(G4S)10 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYTFGGGTKVEIKGGGGSGGGGSGGGGSEVQLVESGGGLVQPGG |
| | SLRLSCAASGETFSDHYMDWVRQAPGKGLASGETFSDHYMDWVRQAPGKGLEW |
| | VGRIRNKASSYTTEYAASVKGRFTISRDDSKNSLYLQMNSLKTEDTAVYYCAREPK |
| | YWIDFDLWGRGTLVTVSS (SEQ ID NO: 373) |
| CDA_(G4S)10 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGGGGGSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYTFGGGTKVEIKGGGGSGGGGSGGGGSEVQLVESGGGLVQPGR |
| | SLRLSCTASGFTFSDHDMNWVRQAPGKGLEWVGRTRNAAGSYTTEYAASVKGRF |
| | TISRDDSKNSLYLQMNSLKTEDTAVYYCAREPKYWIDFDLWGRGTL VTSS (SEQ ID |
| | NO: 374) |
| CDA_(G4S)10 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSEVQLVESGGGLVQPGGSLRLSCAA |
| | SGETFVDHDMDWVRQAPGKGLEWVGRTRNKLGSYTTEYAASVKGRETGRDDSK |
| | NSLYLQMNSLKTEDTAVYYCAREPKYWIDFDLWGRGTLVTVSSGGGGSGGGGSG |
| | GGGSDIQMTQSPSSLSASVGDRVTITCRASQSISSYLNWYQQKPGKAPKLLIYAASS |
| | LQSGVPSRFSGSGSGTDFTLTISSLQPEDFATYYCQQSYIAPYIFGGGTKVEIK (SEQ |
| | ID NO: 375) |
| CDA_(G4S)11 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSDIQMTQSPSSVSASVGDRV |
| | TITCRASQGISSWLAWYQQKPGKAPKLLLIYAASSLQSGVPSRFSGSGSGTDFTLTIS |
| | SLQPEDFATYYCQQTNSFPYTFGGGTKVEIKGGGGSGGGGSGGGGSEVQLLESGG |
| | GLVQPGGSLRLSCAASGETFSNYAMSWVRQAPGKGLEWVSAISGSGGSTYYADSV |
| | KGRFTISRDNSKNTLYLQMNSLRAEDTAVYYCAKGPPTYHTNYYYMDVWGKGTT |
| | VTVSS (SEQ ID NO: 376) |
| CDA_(G4S)11 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSDIQMTQSPSSVSASVGDRV |
| | TITCRASQGISSWLAWYQQKPGKAPKLLIYAASSLQQSGVPSRFSGSGSGTDFTLTI |
| | SSLQPEDFATYYCQQTNSEPYTFGGGTKVEIKGGGGSGGGGSGGGGSEVQLLESG |
| | GGLVQPGGSLRLSCAASGETESSYVMIWVRQAPGKGLEWVSSISGDSVTTYYADS |
| | VKGRFTISRDNSKNTLYLQMNSLRAEDTAVYYCAKGPPTYHTNYYYMDVWGKG |
| | TTVTVSS (SEQ ID NO: 377) |
| CDA_(G4S)11 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGGGGGSGGGGSGGGGSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYTFGGGTKVEIKGGGGSGGGGSGGGGSEVQLVESGGG |
| | LVQPGGSLRLSCAASGETFSDHYMDWVRQAPGKGLASGETFSDHYMDWVRQAP |
| | GKGLEWVGRIRNKASSYTTEYAASVKGRFTISRDDSKNSLYLQMNSLKTEDTAVY |
| | YCAREPKYWIDFDLWGRGTLVTVSS (SEQ ID NO: 378) |
| CDA_(G4S)11 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYTFGGGTKVEIKGGGGSGGGGSGGGGSEVQLVESGGG |
| | LVQPGRSLRLSCTASGFTFSDHDMNWVRQAPGKGLEWVGRTRNAAGSYTTEYAA |
| | SVKGRFTISRDDSKNSLYLQMNSLKTEDTAVYYCAREPKYWIDFDLWGRGTLVTS |
| | S (SEQ ID NO: 379) |
| CDA_(G4S)11 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGGGGGSGGGGSGGGGSEVQLVESGGGLVQPGGSLR |
| | LSCAASGETFVDHDMDWVRQAPGKGLEWVGRTRNKLGSYTTEYAASVKGRETG |
| | RDDSKNSLYLQMNSLKTEDTAVYYCAREPKYWIDFDLWGRGTLVTVSSGGGGSG |
| | GGGSGGGGSDIQMTQSPSSLSASVGDRVTITCRASQSISSYLNWYQQKPGKAPKLLI |
| | YAASSLQSGVPSRFSGSGSGTDFTLTISSLQPEDFATYYCQQSYIAPYIFGGGTKVEI |
| | K (SEQ ID NO: 380) |
| CDA_(G4S)112 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSGGGGSDIQMTQSPSSVSAS |
| | VGDRVTITCRASQGISSWLAWYQQKPGKAPKLLLIYAASSLQSGVPSRFSGSGSGT |
| | DFTLTISSLQPEDFATYYCQQTNSFPYTFGGGTKVEIKGGGGSGGGGSGGGGSEVQ |
| | LLESGGGLVQPGGSLRLSCAASGETFSNYAMSWVRQAPGKGLEWVSAISGSGGST |
| | YYADSVKGRFTISRDNSKNTLYLQMNSLRAEDTAVYYCAKGPPTYHTNYYYMDV |
| | WGKGTTVTVSS (SEQ ID NO: 381) |
| CDA_(G4S)112 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSGGGGSDIQMTQSPSSVSAS |
| | VGDRVTITCRASQGISSWLAWYQQKPGKAPKLLIYAASSLQQSGVPSRFSGSGSGT |
| | DFTLTISSLQPEDFATYYCQQTNSEPYTFGGGTKVEIKGGGGSGGGGSGGGGSEVQ |
| | LLESGGGLVQPGGSLRLSCAASGETESSYVMIWVRQAPGKGLEWVSSISGDSVTTY |
| | YADSVKGRFTISRDNSKNTLYLQMNSLRAEDTAVYYCAKGPPTYHTNYYYMDVW |
| | GKGTTVTVSS (SEQ ID NO: 382) |
| CDA_(G4S)112 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYTFGGGTKVEIKGGGGSGGGGSGGGGSEVQLV |
| | ESGGGLVQPGGSLRLSCAASGETFSDHYMDWVRQAPGKGLASGETFSDHYMDWV |
| | RQAPGKGLEWVGRIRNKASSYTTEYAASVKGRFTISRDDSKNSLYLQMNSLKTED |
| | TAVYYCAREPKYWIDFDLWGRGTLVTVSS (SEQ ID NO: 383) |
| CDA_(G4S)112 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGSGGGGSGGGGS |
| HC | GGGGSGGGGSGGGGSGGGGSGGGGSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYTFGGGTKVEIKGGGGSGGGGSGGGGSEVQLV |
| | ESGGGLVQPGRSLRLSCTASGFTFSDHDMNWVRQAPGKGLEWVGRTRNAAGSYT |
| | TEYAASVKGRFTISRDDSKNSLYLQMNSLKTEDTAVYYCAREPKYWIDFDLWGRG |
| | TLVTSS (SEQ ID NO: 384) |
| CDA_(G4S)112 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| LC_(G4S)3_ | WPVYMTKPDGTYIVMTVQELLPSSFGPEDLQKTQGGGGSGGGGGGGGSGGGGS |
| HC | GGGGSGGGGSGGGGSGGGGSGGGGSGGGGSGGGGSGGGGSEVQLVESGGGLVQ |
| | PGGSLRLSCAASGETFVDHDMDWVRQAPGKGLEWVGRTRNKLGSYTTEYAASVK |
| | GRETGRDDSKNSLYLQMNSLKTEDTAVYYCAREPKYWIDFDLWGRGTLVTVSSG |
| | GGGSGGGGSGGGGSDIQMTQSPSSLSASVGDRVTITCRASQSISSYLNWYQQKPGK |
| | APKLLIYAASSLQSGVPSRFSGSGSGTDFTLTISSLQPEDFATYYCQQSYIAPYIFGG |
| | GTKVEIK (SEQ ID NO: 385) |
| CDA_G4S | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLLESGGGLVQPGG |
| LC | SLRLSCAASGETFSNYAMSWVRQAPGKGLEWVSAISGSGGSTYYADSVKGRFTISR |
| | DNSKNTLYLQMNSLRAEDTAVYYCAKGPPTYHTNYYYMDVWGKGTTVTVSSGG |
| | GGSGGGGSGGGGSDIQMTQSPSSVSASVGDRVTITCRASQGISSWLAWYQQKPGK |
| | APKLLLIYAASSLQSGVPSRFSGSGSGTDFTLTISSLQPEDFATYYCQQTNSFPYTFG |
| | GGTKVEIK (SEQ ID NO: 386) |
| CDA_G4S | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLLESGGGLVQPGG |
| LC | SLRLSCAASGETESSYVMIWVRQAPGKGLEWVSSISGDSVTTYYADSVKGRFTISR |
| | DNSKNTLYLQMNSLRAEDTAVYYCAKGPPTYHTNYYYMDVWGKGTTVTVSSGG |
| | GGSGGGGSGGGGSDIQMTQSPSSVSASVGDRVTITCRASQGISSWLAWYQQKPGK |
| | APKLLIYAASSLQQSGVPSRFSGSGSGTDFTLTISSLQPEDFATYYCQQTNSEPYTFG |
| | GGTKVEIK (SEQ ID NO: 387) |
| CDA_G4S | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLVESGGGLVQPGG |
| LC | SLRLSCAASGETFSDHYMDWVRQAPGKGLASGETFSDHYMDWVRQAPGKGL |
| | EWVGRIRNKASSYTTEYAASVKGRFTISRDDSKNSLYLQMNSLKTEDTAVYYCAR |
| | EPKYWIDFDLWGRGTLVTVSSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYTFGGGTKVEIK (SEQ ID NO: 388) |
| CDA_G4S | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSEVQLVESGGGLVQPGR |
| LC | SLRLSCTASGFTFSDHDMNWVRQAPGKGLEWVGRTRNAAGSYTTEYAASVKGRF |
| | TISRDDSKNSLYLQMNSLKTEDTAVYYCAREPKYWIDFDLWGRGTLVTSSGGGGS |
| | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIK (SEQ ID NO: 389) |
| CDA_G4S | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSDIQMTQSPSSLSASVG |
| LC | DRVTITCRASQSISSYLNWYQQKPGKAPKLLIYAASSLQSGVPSRFSGSGSGTDFTL |
| | TISSLQPEDFATYYCQQSYIAPYIFGGGTKVEIKGGGGSGGGGSGGGGSEVQLVESG |
| | GGLVQPGGSLRLSCAASGETFVDHDMDWVRQAPGKGLEWVGRTRNKLGSYTTE |
| | YAASVKGRETGRDDSKNSLYLQMNSLKTEDTAVYYCAREPKYWIDFDLWGRGTL |
| | VTVSS (SEQ ID NO: 390) |
| CDA_(G4S)3 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLLE |
| LC | SGGGLVQPGGSLRLSCAASGETFSNYAMSWVRQAPGKGLEWVSAISGSGGSTYYA |
| | DSVKGRFTISRDNSKNTLYLQMNSLRAEDTAVYYCAKGPPTYHTNYYYMDVWGK |
| | GTTVTVSSGGGGSGGGGSGGGGSDIQMTQSPSSVSASVGDRVTITCRASQGISSWL |
| | AWYQQKPGKAPKLLLIYAASSLQSGVPSRFSGSGSGTDFTLTISSLQPEDFATYYCQ |
| | QTNSFPYTFGGGTKVEIK (SEQ ID NO: 391) |
| CDA_(G4S)3 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLLE |
| LC | SGGGLVQPGGSLRLSCAASGETESSYVMIWVRQAPGKGLEWVSSISGDSVTTYYA |
| | DSVKGRFTISRDNSKNTLYLQMNSLRAEDTAVYYCAKGPPTYHTNYYYMDVWGK |
| | GTTVTVSSGGGGGGGGSGGGGSDIQMTQSPSSVSASVGDRVTITCRASQGISSWL |
| | AWYQQKPGKAPKLLIYAASSLQQSGVPSRFSGSGSGTDFTLTISSLQPEDFATYYCQ |
| | QTNSEPYTFGGGTKVEIK (SEQ ID NO: 392) |
| CDA_(G4S)3 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLV |
| LC | ESGGGLVQPGGSLRLSCAASGETFSDHYMDWVRQAPGKGLASGETFSDHYMDWV |
| | RQAPGKGLEWVGRIRNKASSYTTEYAASVKGRFTISRDDSKNSLYLQMNSLKTED |
| | TAVYYCAREPKYWIDFDLWGRGTLVTVSSGGGGSGGGGSGGGGSDIQMTQSPSSL |
| | SASVGDRVTITCRASQSISSYLNWYQQKPGKAPKLLIYAASSLQSGVPSRFSGSGSG |
| | TDFTLTISSLQPEDFATYYCQQSYIAPYTFGGGTKVEIK (SEQ ID NO: 393) |
| CDA_(G4S)3 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSEVQLV |
| LC | ESGGGLVQPGRSLRLSCTASGFTFSDHDMNWVRQAPGKGLEWVGRTRNAAGSYT |
| | TEYAASVKGRFTISRDDSKNSLYLQMNSLKTEDTAVYYCAREPKYWIDFDLWGRG |
| | TLVTSSGGGGSGGGGSGGGGSDIQMTQSPSSLSASVGDRVTITCRASQSISSYLNW |
| | YQQKPGKAPKLLIYAASSLQSGVPSRFSGSGSGTDFTLTISSLQPEDFATYYCQQSYI |
| | APYTFGGGTKVEIK (SEQ ID NO: 394) |
| CDA_(G4S)3 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSDIQMT |
| LC | QSPSSLSASVGDRVTITCRASQSISSYLNWYQQKPGKAPKLLIYAASSLQSGVPSRFS |
| | GSGSGTDFTLTISSLQPEDFATYYCQQSYIAPYIFGGGTKVEIKGGGGSGGGGSGGG |
| | GSEVQLVESGGGLVQPGGSLRLSCAASGETFVDHDMDWVRQAPGKGLEWVGRTR |
| | NKLGSYTTEYAASVKGRETGRDDSKNSLYLQMNSLKTEDTAVYYCAREPKYWID |
| | FDLWGRGTLVTVSS (SEQ ID NO: 395) |
| CDA_(G4S)4 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | EVQLLESGGGLVQPGGSLRLSCAASGETFSNYAMSWVRQAPGKGLEWVSAISGSG |
| | GSTYYADSVKGRFTISRDNSKNTLYLQMNSLRAEDTAVYYCAKGPPTYHTNYYY |
| | MDVWGKGTTVTVSSGGGGSGGGGSGGGGSDIQMTQSPSSVSASVGDRVTITCRAS |
| | QGISSWLAWYQQKPGKAPKLLLIYAASSLQSGVPSRFSGSGSGTDFTLTISSLQPED |
| | FATYYCQQTNSFPYTFGGGTKVEIK (SEQ ID NO: 396) |
| CDA_(G4S)4 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | EVQLLESGGGLVQPGGSLRLSCAASGETESSYVMIWVRQAPGKGLEWVSSISGDSV |
| | TTYYADSVKGRFTISRDNSKNTLYLQMNSLRAEDTAVYYCAKGPPTYHTNYYYM |
| | DVWGKGTTVTVSSGGGGSGGGGSGGGGSDIQMTQSPSSVSASVGDRVTITCRASQ |
| | GISSWLAWYQQKPGKAPKLLIYAASSLQQSGVPSRFSGSGSGTDFTLTISSLQPEDF |
| | ATYYCQQTNSEPYTFGGGTKVEIK (SEQ ID NO: 397) |
| CDA_(G4S)4 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | EVQLVESGGGLVQPGGSLRLSCAASGETFSDHYMDWVRQAPGKGLASGETFSDH |
| | YMDWVRQAPGKGLEWVGRIRNKASSYTTEYAASVKGRFTISRDDSKNSLYLQMN |
| | SLKTEDTAVYYCAREPKYWIDFDLWGRGTLVTVSSGGGGSGGGGSGGGGSDIQM |
| | TQSPSSLSASVGDRVTITCRASQSISSYLNWYQQKPGKAPKLLIYAASSLQSGVPSR |
| | FSGSGSGTDFTLTISSLQPEDFATYYCQQSYIAPYTFGGGTKVEIK (SEQ ID NO: |
| | 398) |
| CDA_(G4S)4 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | EVQLVESGGGLVQPGRSLRLSCTASGFTFSDHDMNWVRQAPGKGLEWVGRTRNA |
| | AGSYTTEYAASVKGRFTISRDDSKNSLYLQMNSLKTEDTAVYYCAREPKYWIDFD |
| | LWGRGTLVTSSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYTFGGGTKVEIK (SEQ ID NO: 399) |
| CDA_(G4S)4 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYIFGGGTKVEIKGGGGSGGG |
| | GSGGGGSEVQLVESGGGLVQPGGSLRLSCAASGETFVDHDMDWVRQAPGKGLE |
| | WVGRTRNKLGSYTTEYAASVKGRETGRDDSKNSLYLQMNSLKTEDTAVYYCARE |
| | PKYWIDFDLWGRGTLVTVSS (SEQ ID NO: 400) |
| CDA_(G4S)5 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSEVQLLESGGGLVQPGGSLRLSCAASGETFSNYAMSWVRQAPGKGLEWVS |
| | AISGSGGSTYYADSVKGRFTISRDNSKNTLYLQMNSLRAEDTAVYYCAKGPPTYH |
| | TNYYYMDVWGKGTTVTVSSGGGGSGGGGSGGGGSDIQMTQSPSSVSASVGDRVT |
| | ITCRASQGISSWLAWYQQKPGKAPKLLLIYAASSLQSGVPSRFSGSGSGTDFTLTISS |
| | LQPEDFATYYCQQTNSFPYTFGGGTKVEIK (SEQ ID NO: 401) |
| CDA_(G4S)5 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSEVQLLESGGGLVQPGGSLRLSCAASGETESSYVMIWVRQAPGKGLEWVSS |
| | ISGDSVTTYYADSVKGRFTISRDNSKNTLYLQMNSLRAEDTAVYYCAKGPPTYHT |
| | NYYYMDVWGKGTTVTVSSGGGGSGGGGSGGGGSDIQMTQSPSSVSASVGDRVTI |
| | TCRASQGISSWLAWYQQKPGKAPKLLIYAASSLQQSGVPSRFSGSGSGTDFTLTISS |
| | LQPEDFATYYCQQTNSEPYTFGGGTKVEIK (SEQ ID NO: 402) |
| CDA_(G4S)5 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSEVQLVESGGGLVQPGGSLRLSCAASGETFSDHYMDWVRQAPGKGLASGE |
| | TFSDHYMDWVRQAPGKGLEWVGRIRNKASSYTTEYAASVKGRFTISRDDSKNSLY |
| | LQMNSLKTEDTAVYYCAREPKYWIDFDLWGRGTLVTVSSGGGGSGGGGSGGGGS |
| | DIQMTQSPSSLSASVGDRVTITCRASQSISSYLNWYQQKPGKAPKLLIYAASSLQSG |
| | VPSRFSGSGSGTDFTLTISSLQPEDFATYYCQQSYIAPYTFGGGTKVEIK (SEQ ID |
| | NO: 403) |
| CDA_(G4S)5 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSEVQLVESGGGLVQPGRSLRLSCTASGFTFSDHDMNWVRQAPGKGLEWVG |
| | RTRNAAGSYTTEYAASVKGRFTISRDDSKNSLYLQMNSLKTEDTAVYYCAREPKY |
| | WIDFDLWGRGTLVTSSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYTFGGGTKVEIK (SEQ ID NO: 404) |
| CDA_(G4S)5 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYIFGGGTKVEIKGGG |
| | GSGGGGSGGGGSEVQLVESGGGLVQPGGSLRLSCAASGETFVDHDMDWVRQAPG |
| | KGLEWVGRTRNKLGSYTTEYAASVKGRETGRDDSKNSLYLQMNSLKTEDTAVYY |
| | CAREPKYWIDFDLWGRGTLVTVSS (SEQ ID NO: 405) |
| CDA_(G4S)6 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGGGGGSEVQLLESGGGLVQPGGSLRLSCAASGETFSNYAMSWVRQAPGKG |
| | LEWVSAISGSGGSTYYADSVKGRFTISRDNSKNTLYLQMNSLRAEDTAVYYCAKG |
| | PPTYHTNYYYMDVWGKGTTVTVSSGGGGSGGGGSGGGGSDIQMTQSPSSVSASV |
| | GDRVTITCRASQGISSWLAWYQQKPGKAPKLLLIYAASSLQSGVPSRFSGSGSGTD |
| | FTLTISSLQPEDFATYYCQQTNSFPYTFGGGTKVEIK (SEQ ID NO: 406) |
| CDA_(G4S)6 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSEVQLLESGGGLVQPGGSLRLSCAASGETESSYVMIWVRQAPGKGL |
| | EWVSSISGDSVTTYYADSVKGRFTISRDNSKNTLYLQMNSLRAEDTAVYYCAKGP |
| | PTYHTNYYYMDVWGKGTTVTVSSGGGGSGGGGSGGGGSDIQMTQSPSSVSASVG |
| | DRVTITCRASQGISSWLAWYQQKPGKAPKLLIYAASSLQQSGVPSRFSGSGSGTDF |
| | TLTISSLQPEDFATYYCQQTNSEPYTFGGGTKVEIK (SEQ ID NO: 407) |
| CDA_(G4S)6 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSEVQLVESGGGLVQPGGSLRLSCAASGETFSDHYMDWVRQAPGKG |
| | LASGETFSDHYMDWVRQAPGKGLEWVGRIRNKASSYTTEYAASVKGRFTISRDDS |
| | KNSLYLQMNSLKTEDTAVYYCAREPKYWIDFDLWGRGTLVTVSSGGGGSGGGGS |
| | GGGGSDIQMTQSPSSLSASVGDRVTITCRASQSISSYLNWYQQKPGKAPKLLIYAAS |
| | SLQSGVPSRFSGSGSGTDFTLTISSLQPEDFATYYCQQSYIAPYTFGGGTKVEIK |
| | (SEQ ID NO: 408) |
| CDA_(G4S)6 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSEVQLVESGGGLVQPGRSLRLSCTASGFTFSDHDMNWVRQAPGKG |
| | LEWVGRTRNAAGSYTTEYAASVKGRFTISRDDSKNSLYLQMNSLKTEDTAVYYC |
| | AREPKYWIDFDLWGRGTLVTSSGGGGSGGGGSGGGGSDIQMTQSPSSLSASVGDR |
| | VTITCRASQSISSYLNWYQQKPGKAPKLLIYAASSLQSGVPSRFSGSGSGTDFTLTIS |
| | SLQPEDFATYYCQQSYIAPYTFGGGTKVEIK (SEQ ID NO: 409) |
| CDA_(G4S)6 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYIFGGGTKV |
| | EIKGGGGSGGGGSGGGGSEVQLVESGGGLVQPGGSLRLSCAASGETFVDHDMDW |
| | VRQAPGKGLEWVGRTRNKLGSYTTEYAASVKGRETGRDDSKNSLYLQMNSLKTE |
| | DTAVYYCAREPKYWIDFDLWGRGTLVTVSS (SEQ ID NO: 410) |
| CDA_(G4S)7 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSEVQLLESGGGLVQPGGSLRLSCAASGETFSNYAMSWVRQ |
| | APGKGLEWVSAISGSGGSTYYADSVKGRFTISRDNSKNTLYLQMNSLRAEDTAVY |
| | YCAKGPPTYHTNYYYMDVWGKGTTVTVSSGGGGSGGGGSGGGGSDIQMTQSPSS |
| | VSASVGDRVTITCRASQGISSWLAWYQQKPGKAPKLLLIYAASSLQSGVPSRFSGS |
| | GSGTDFTLTISSLQPEDFATYYCQQTNSFPYTFGGGTKVEIK (SEQ ID NO: 411) |
| CDA_(G4S)7 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSEVQLLESGGGLVQPGGSLRLSCAASGETESSYVMIWVRQA |
| | PGKGLEWVSSISGDSVTTYYADSVKGRFTISRDNSKNTLYLQMNSLRAEDTAVYY |
| | CAKGPPTYHTNYYYMDVWGKGTTVTVSSGGGGSGGGGSGGGGSDIQMTQSPSSV |
| | SASVGDRVTITCRASQGISSWLAWYQQKPGKAPKLLIYAASSLQQSGVPSRFSGSG |
| | SGTDFTLTISSLQPEDFATYYCQQTNSEPYTFGGGTKVEIK (SEQ ID NO: 412) |
| CDA_(G4S)7 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSEVQLVESGGGLVQPGGSLRLSCAASGETFSDHYMDWVRQ |
| | APGKGLASGETFSDHYMDWVRQAPGKGLEWVGRIRNKASSYTTEYAASVKGRFT |
| | ISRDDSKNSLYLQMNSLKTEDTAVYYCAREPKYWIDFDLWGRGTLVTVSSGGGGS |
| | GGGGSGGGGSDIQMTQSPSSLSASVGDRVTITCRASQSISSYLNWYQQKPGKAPKL |
| | LIYAASSLQSGVPSRFSGSGSGTDFTLTISSLQPEDFATYYCQQSYIAPYTFGGGTKV |
| | EIK (SEQ ID NO: 413) |
| CDA_(G4S)7 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSEVQLVESGGGLVQPGRSLRLSCTASGFTFSDHDMNWVRQ |
| | APGKGLEWVGRTRNAAGSYTTEYAASVKGRFTISRDDSKNSLYLQMNSLKTEDTA |
| | VYYCAREPKYWIDFDLWGRGTLVTSSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYTFGGGTKVEIK (SEQ ID NO: 414) |
| CDA_(G4S)7 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGGGGGSGGGGSDIQMTQSPSSLSASVGDRVTITCRASQSISSYLNWYQQKPG |
| | KAPKLLIYAASSLQSGVPSRFSGSGSGTDFTLTISSLQPEDFATYYCQQSYIAPYIFG |
| | GGTKVEIKGGGGSGGGGSGGGGSEVQLVESGGGLVQPGGSLRLSCAASGETFVDH |
| | DMDWVRQAPGKGLEWVGRTRNKLGSYTTEYAASVKGRETGRDDSKNSLYLQMN |
| | SLKTEDTAVYYCAREPKYWIDFDLWGRGTLVTVSS (SEQ ID NO: 415) |
| CDA_(G4S)8 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSEVQLLESGGGLVQPGGSLRLSCAASGETFSNYAMS |
| | WVRQAPGKGLEWVSAISGSGGSTYYADSVKGRFTISRDNSKNTLYLQMNSLRAED |
| | TAVYYCAKGPPTYHTNYYYMDVWGKGTTVTVSSGGGGSGGGGSGGGGSDIQMT |
| | QSPSSVSASVGDRVTITCRASQGISSWLAWYQQKPGKAPKLLLIYAASSLQSGVPSR |
| | FSGSGSGTDFTLTISSLQPEDFATYYCQQTNSFPYTFGGGTKVEIK (SEQ ID NO: |
| | 416) |
| CDA_(G4S)8 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSEVQLLESGGGLVQPGGSLRLSCAASGETESSYVMI |
| | WVRQAPGKGLEWVSSISGDSVTTYYADSVKGRFTISRDNSKNTLYLQMNSLRAED |
| | TAVYYCAKGPPTYHTNYYYMDVWGKGTTVTVSSGGGGSGGGGSGGGGSDIQMT |
| | QSPSSVSASVGDRVTITCRASQGISSWLAWYQQKPGKAPKLLIYAASSLQQSGVPS |
| | RFSGSGSGTDFTLTISSLQPEDFATYYCQQTNSEPYTFGGGTKVEIK (SEQ ID NO: |
| | 417) |
| CDA_(G4S)8 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSEVQLVESGGGLVQPGGSLRLSCAASGETFSDHYM |
| | DWVRQAPGKGLASGETFSDHYMDWVRQAPGKGLEWVGRIRNKASSYTTEYAAS |
| | VKGRFTISRDDSKNSLYLQMNSLKTEDTAVYYCAREPKYWIDFDLWGRGTLVTVS |
| | SGGGGSGGGGSGGGGSDIQMTQSPSSLSASVGDRVTITCRASQSISSYLNWYQQKP |
| | GKAPKLLIYAASSLQSGVPSRFSGSGSGTDFTLTISSLQPEDFATYYCQQSYIAPYTF |
| | GGGTKVEIK (SEQ ID NO: 418) |
| CDA_(G4S)8 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGGGGGSGGGGSEVQLVESGGGLVQPGRSLRLSCTASGFTFSDHDM |
| | NWVRQAPGKGLEWVGRTRNAAGSYTTEYAASVKGRFTISRDDSKNSLYLQMNSL |
| | KTEDTAVYYCAREPKYWIDFDLWGRGTLVTSSGGGGSGGGGSGGGGSDIQMTQS |
| | PSSLSASVGDRVTITCRASQSISSYLNWYQQKPGKAPKLLIYAASSLQSGVPSRFSGS |
| | GSGTDFTLTISSLQPEDFATYYCQQSYIAPYTFGGGTKVEIK (SEQ ID NO: 419) |
| CDA_(G4S)8 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYIFGGGTKVEIKGGGGSGGGGSGGGGSEVQLVESGGGLVQPGGSLRLSCAASGET |
| | FVDHDMDWVRQAPGKGLEWVGRTRNKLGSYTTEYAASVKGRETGRDDSKNSLY |
| | LQMNSLKTEDTAVYYCAREPKYWIDFDLWGRGTLVTVSS (SEQ ID NO: 420) |
| CDA_(G4S)9 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSEVQLLESGGGLVQPGGSLRLSCAASGETFS |
| | NYAMSWVRQAPGKGLEWVSAISGSGGSTYYADSVKGRFTISRDNSKNTLYLQMN |
| | SLRAEDTAVYYCAKGPPTYHTNYYYMDVWGKGTTVTVSSGGGGSGGGGSGGGG |
| | SDIQMTQSPSSVSASVGDRVTITCRASQGISSWLAWYQQKPGKAPKLLLIYAASSL |
| | QSGVPSRFSGSGSGTDFTLTISSLQPEDFATYYCQQTNSFPYTFGGGTKVEIK (SEQ |
| | ID NO: 421) |
| CDA_(G4S)9 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSEVQLLESGGGLVQPGGSLRLSCAASGETES |
| | SYVMIWVRQAPGKGLEWVSSISGDSVTTYYADSVKGRFTISRDNSKNTLYLQMNS |
| | LRAEDTAVYYCAKGPPTYHTNYYYMDVWGKGTTVTVSSGGGGSGGGGSGGGGS |
| | DIQMTQSPSSVSASVGDRVTITCRASQGISSWLAWYQQKPGKAPKLLIYAASSLQQ |
| | SGVPSRFSGSGSGTDFTLTISSLQPEDFATYYCQQTNSEPYTFGGGTKVEIK (SEQ ID |
| | NO: 422) |
| CDA_(G4S)9 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSEVQLVESGGGLVQPGGSLRLSCAASGETFS |
| | DHYMDWVRQAPGKGLASGETFSDHYMDWVRQAPGKGLEWVGRIRNKASSYTTE |
| | YAASVKGRFTISRDDSKNSLYLQMNSLKTEDTAVYYCAREPKYWIDFDLWGRGTL |
| | VTVSSGGGGGGGGSGGGGSDIQMTQSPSSLSASVGDRVTITCRASQSISSYLNWY |
| | QQKPGKAPKLLIYAASSLQSGVPSRFSGSGSGTDFTLTISSLQPEDFATYYCQQSYIA |
| | PYTFGGGTKVEIK (SEQ ID NO: 423) |
| CDA_(G4S)9 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSEVQLVESGGGLVQPGRSLRLSCTASGFTFS |
| | DHDMNWVRQAPGKGLEWVGRTRNAAGSYTTEYAASVKGRFTISRDDSKNSLYLQ |
| | MNSLKTEDTAVYYCAREPKYWIDFDLWGRGTLVTSSGGGGSGGGGSGGGGSDIQ |
| | MTQSPSSLSASVGDRVTITCRASQSISSYLNWYQQKPGKAPKLLIYAASSLQSGVPS |
| | RFSGSGSGTDFTLTISSLQPEDFATYYCQQSYIAPYTFGGGTKVEIK (SEQ ID NO: |
| | 424) |
| CDA_(G4S)9 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSDIQMTQSPSSLSASVGDRVTITCRASQSISS |
| | YLNWYQQKPGKAPKLLIYAASSLQSGVPSRFSGSGSGTDFTLTISSLQPEDFATYYC |
| | QQSYIAPYIFGGGTKVEIKGGGGSGGGGSGGGGSEVQLVESGGGLVQPGGSLRLSC |
| | AASGETFVDHDMDWVRQAPGKGLEWVGRTRNKLGSYTTEYAASVKGRETGRDD |
| | SKNSLYLQMNSLKTEDTAVYYCAREPKYWIDFDLWGRGTLVTVSS (SEQ ID NO: |
| | 425) |
| CDA_(G4S)10 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSEVQLLESGGGLVQPGGSLRLSCAA |
| | SGETFSNYAMSWVRQAPGKGLEWVSAISGSGGSTYYADSVKGRFTISRDNSKNTL |
| | YLQMNSLRAEDTAVYYCAKGPPTYHTNYYYMDVWGKGTTVTVSSGGGGSGGGG |
| | SGGGGSDIQMTQSPSSVSASVGDRVTITCRASQGISSWLAWYQQKPGKAPKLLLIY |
| | AASSLQSGVPSRFSGSGSGTDFTLTISSLQPEDFATYYCQQTNSFPYTFGGGTKVEIK |
| | (SEQ ID NO: 426) |
| CDA_(G4S)10 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSEVQLLESGGGLVQPGGSLRLSCAA |
| | SGETESSYVMIWVRQAPGKGLEWVSSISGDSVTTYYADSVKGRFTISRDNSKNTLY |
| | LQMNSLRAEDTAVYYCAKGPPTYHTNYYYMDVWGKGTTVTVSSGGGGSGGGGS |
| | GGGGSDIQMTQSPSSVSASVGDRVTITCRASQGISSWLAWYQQKPGKAPKLLIYAA |
| | SSLQQSGVPSRFSGSGSGTDFTLTISSLQPEDFATYYCQQTNSEPYTFGGGTKVEIK |
| | (SEQ ID NO: 427) |
| CDA_(G4S)10 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSEVQLVESGGGLVQPGGSLRLSCAA |
| | SGETFSDHYMDWVRQAPGKGLASGETFSDHYMDWVRQAPGKGLEWVGRIRNKA |
| | SSYTTEYAASVKGRFTISRDDSKNSLYLQMNSLKTEDTAVYYCAREPKYWIDFDL |
| | WGRGTLVTVSSGGGGSGGGGSGGGGSDIQMTQSPSSLSASVGDRVTITCRASQSIS |
| | SYLNWYQQKPGKAPKLLIYAASSLQSGVPSRFSGSGSGTDFTLTISSLQPEDFATYY |
| | CQQSYIAPYTFGGGTKVEIK (SEQ ID NO: 428) |
| CDA_(G4S)10 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSEVQLVESGGGLVQPGRSLRLSCTA |
| | SGFTFSDHDMNWVRQAPGKGLEWVGRTRNAAGSYTTEYAASVKGRFTISRDDSK |
| | NSLYLQMNSLKTEDTAVYYCAREPKYWIDFDLWGRGTLVTSSGGGGSGGGGSGG |
| | GGSDIQMTQSPSSLSASVGDRVTITCRASQSISSYLNWYQQKPGKAPKLLIYAASSL |
| | QSGVPSRFSGSGSGTDFTLTISSLQPEDFATYYCQQSYIAPYTFGGGTKVEIK (SEQ |
| | ID NO: 429) |
| CDA_(G4S)10 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSDIQMTQSPSSLSASVGDRVTITCRA |
| | SQSISSYLNWYQQKPGKAPKLLIYAASSLQSGVPSRFSGSGSGTDFTLTISSLQPEDF |
| | ATYYCQQSYIAPYIFGGGTKVEIKGGGGSGGGGSGGGGSEVQLVESGGGLVQPGG |
| | SLRLSCAASGETFVDHDMDWVRQAPGKGLEWVGRTRNKLGSYTTEYAASVKGRE |
| | TGRDDSKNSLYLQMNSLKTEDTAVYYCAREPKYWIDFDLWGRGTLVTVSS (SEQ |
| | ID NO: 430) |
| CDA_(G4S)11 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSEVQLLESGGGLVQPGGSLR |
| | LSCAASGETFSNYAMSWVRQAPGKGLEWVSAISGSGGSTYYADSVKGRFTISRDN |
| | SKNTLYLQMNSLRAEDTAVYYCAKGPPTYHTNYYYMDVWGKGTTVTVSSGGGG |
| | SGGGGSGGGGSDIQMTQSPSSVSASVGDRVTITCRASQGISSWLAWYQQKPGKAP |
| | KLLLIYAASSLQSGVPSRFSGSGSGTDFTLTISSLQPEDFATYYCQQTNSFPYTFGGG |
| | TKVEIK (SEQ ID NO: 431) |
| CDA_(G4S)11 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGGGGGSGGGGSGGGGSGGGGSGGGGSEVQLLESGGGLVQPGGSLR |
| | LSCAASGETESSYVMIWVRQAPGKGLEWVSSISGDSVTTYYADSVKGRFTISRDNS |
| | KNTLYLQMNSLRAEDTAVYYCAKGPPTYHTNYYYMDVWGKGTTVTVSSGGGGS |
| | GGGGSGGGGSDIQMTQSPSSVSASVGDRVTITCRASQGISSWLAWYQQKPGKAPK |
| | LLIYAASSLQQSGVPSRFSGSGSGTDFTLTISSLQPEDFATYYCQQTNSEPYTFGGGT |
| | KVEIK (SEQ ID NO: 432) |
| CDA_(G4S)11 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSEVQLVESGGGLVQPGGSLR |
| | LSCAASGETFSDHYMDWVRQAPGKGLASGETFSDHYMDWVRQAPGKGLEWVGR |
| | IRNKASSYTTEYAASVKGRFTISRDDSKNSLYLQMNSLKTEDTAVYYCAREPKYWI |
| | DFDLWGRGTLVTVSSGGGGSGGGGSGGGGSDIQMTQSPSSLSASVGDRVTITCRAS |
| | QSISSYLNWYQQKPGKAPKLLIYAASSLQSGVPSRFSGSGSGTDFTLTISSLQPEDFA |
| | TYYCQQSYIAPYTFGGGTKVEIK (SEQ ID NO: 433) |
| CDA_(G4S)11 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSEVQLVESGGGLVQPGRSLR |
| | LSCTASGFTFSDHDMNWVRQAPGKGLEWVGRTRNAAGSYTTEYAASVKGRFTIS |
| | RDDSKNSLYLQMNSLKTEDTAVYYCAREPKYWIDFDLWGRGTLVTSSGGGGSGG |
| | GGSGGGGSDIQMTQSPSSLSASVGDRVTITCRASQSISSYLNWYQQKPGKAPKLLIY |
| | AASSLQSGVPSRFSGSGSGTDFTLTISSLQPEDFATYYCQQSYIAPYTFGGGTKVEIK |
| | (SEQ ID NO: 434) |
| CDA_(G4S)11 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSDIQMTQSPSSLSASVGDRV |
| | TITCRASQSISSYLNWYQQKPGKAPKLLIYAASSLQSGVPSRFSGSGSGTDFTLTISS |
| | LQPEDFATYYCQQSYIAPYIFGGGTKVEIKGGGGSGGGGSGGGGSEVQLVESGGGL |
| | VQPGGSLRLSCAASGETFVDHDMDWVRQAPGKGLEWVGRTRNKLGSYTTEYAAS |
| | VKGRETGRDDSKNSLYLQMNSLKTEDTAVYYCAREPKYWIDFDLWGRGTLVTVS |
| | S (SEQ ID NO: 435) |
| CDA_(G4S)112 | MAQKRPACTLKPECVQQLLVCSQEAKKSAYCPYSHFPVGAALLTQEGRIFKGCNIE |
| linker_Ab58_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSGGGGSEVQLLESGGGLVQP |
| | GGSLRLSCAASGETFSNYAMSWVRQAPGKGLEWVSAISGSGGSTYYADSVKGRFT |
| | ISRDNSKNTLYLQMNSLRAEDTAVYYCAKGPPTYHTNYYYMDVWGKGTTVTVSS |
| | GGGGSGGGGSGGGGSDIQMTQSPSSVSASVGDRVTITCRASQGISSWLAWYQQKP |
| | GKAPKLLLIYAASSLQSGVPSRFSGSGSGTDFTLTISSLQPEDFATYYCQQTNSFPYT |
| | FGGGTKVEIK (SEQ ID NO: 436) |
| CDA_(G4S)112 | MAQKRPACTLKPECVQQLLVCSQEAKKSAYCPYSHFPVGAALLTQEGRIFKGCNIE |
| linker_Ab61_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSGGGGSEVQLLESGGGLVQP |
| | GGSLRLSCAASGETESSYVMIWVRQAPGKGLEWVSSISGDSVTTYYADSVKGRFTI |
| | SRDNSKNTLYLQMNSLRAEDTAVYYCAKGPPTYHTNYYYMDVWGKGTTVTVSS |
| | GGGGSGGGGSGGGGSDIQMTQSPSSVSASVGDRVTITCRASQGISSWLAWYQQKP |
| | GKAPKLLIYAASSLQQSGVPSRFSGSGSGTDFTLTISSLQPEDFATYYCQQTNSEPYT |
| | FGGGTKVEIK (SEQ ID NO: 437) |
| CDA_(G4S)112 | MAQKRPACTLKPECVQQLLVCSQEAKKSAYCPYSHFPVGAALLTQEGRIFKGCNIE |
| linker_Ab66_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSGGGGSEVQLVESGGGLVQ |
| | PGGSLRLSCAASGETFSDHYMDWVRQAPGKGLASGETFSDHYMDWVRQAPGKG |
| | LEWVGRIRNKASSYTTEYAASVKGRFTISRDDSKNSLYLQMNSLKTEDTAVYYCA |
| | REPKYWIDFDLWGRGTLVTVSSGGGGSGGGGSGGGGSDIQMTQSPSSLSASVGDR |
| | VTITCRASQSISSYLNWYQQKPGKAPKLLIYAASSLQSGVPSRFSGSGSGTDFTLTIS |
| | SLQPEDFATYYCQQSYIAPYTFGGGTKVEIK (SEQ ID NO: 438) |
| CDA_(G4S)112 | MAQKRPACTLKPECVQQLLVCSQEAKKSAYCPYSHFPVGAALLTQEGRIFKGCNIE |
| linker_Ab68_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGSGGGGSGGGGSGGGGSGGGGSEVQLVESGGGLVQ |
| | PGRSLRLSCTASGFTFSDHDMNWVRQAPGKGLEWVGRTRNAAGSYTTEYAASVK |
| | GRFTISRDDSKNSLYLQMNSLKTEDTAVYYCAREPKYWIDFDLWGRGTLVTSSGG |
| | GGSGGGGSGGGGSDIQMTQSPSSLSASVGDRVTITCRASQSISSYLNWYQQKPGKA |
| | PKLLIYAASSLQSGVPSRFSGSGSGTDFTLTISSLQPEDFATYYCQQSYIAPYTFGGG |
| | TKVEIK (SEQ ID NO: 439) |
| CDA_(G4S)112 | MAQKRPACTLKPECVQQLLVCSQEAKKSAYCPYSHFPVGAALLTQEGRIFKGCNIE |
| linker_Ab69_ | NACYPLGICAERTAIQKAVSEGYKDFRAIAIASDMQDDFISPCGACRQVMREFGTN |
| HC_(G4S)3_ | WPVYMTKPDGTYIVMTVQELLPSSFGPEDLQKTQGGGGSGGGGSGGGGSGGGGS |
| LC | GGGGSGGGGSGGGGSGGGGGGGGSGGGGSGGGGSGGGGSDIQMTQSPSSLSAS |
| | VGDRVTITCRASQSISSYLNWYQQKPGKAPKLLIYAASSLQSGVPSRFSGSGSGTDF |
| | TLTISSLQPEDFATYYCQQSYIAPYIFGGGTKVEIKGGGGSGGGGSGGGGSEVQLVE |
| | SGGGLVQPGGSLRLSCAASGETFVDHDMDWVRQAPGKGLEWVGRTRNKLGSYTT |
| | EYAASVKGRETGRDDSKNSLYLQMNSLKTEDTAVYYCAREPKYWIDFDLWGRGT |
| | LVTVSS (SEQ ID NO: 440) |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The publications listed in the application, to the extent that they provide exemplary procedural or other details supplementary to those set forth herein, are specifically incorporated herein by reference.

What is claimed is:

1. A composition comprising an antigen-binding protein conjugated to a protection molecule, wherein the protection molecule comprises a molecule capable of neutralizing a cytotoxic agent.

2. The composition of claim 1, wherein the antigen-binding protein is an antibody or functional fragment thereof.

3. The composition of claim 1 or 2, wherein the antigen-binding protein is capable of binding to an antigen expressed on a healthy cell.

4. The composition of claim 3, wherein the healthy cell is a rapidly dividing healthy cell.

5. The composition of claim 3 or 4, wherein the healthy cell is a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell.

6. The composition of any one of claims 1-5, wherein the antigen-binding protein is capable of binding to CD34.

7. The composition of any one of claims 1-5, wherein the antigen-binding protein is not capable of binding to CD34.

8. The composition of any one of claims 1-5, wherein the antigen-binding protein is capable of binding to CD117.

9. The composition of any one of claims 1-5, wherein the antigen-binding protein is not capable of binding to CD117.

10. The composition of any one of claims 1-5, wherein the antigen-binding protein comprises a heavy chain amino acid sequence provided in Table 1 and a light chain amino acid sequence provided in Table 1, or a functional fragment thereof.

11. The composition of any one of claims 1-10, wherein the antigen-binding protein and/or protein molecule comprises a purification tag.

12. The composition of claim 11, wherein the purification tag comprises a 6×Histidine tag.

13. The composition of claim 11 or 12, wherein the purification tag comprises a protease cleavage sequence.

14. The composition of any one of claims 1-13, wherein the antigen-binding protein does not comprise an Fc region.

15. The composition of any one of claims 1-14, wherein protection molecule comprises an enzyme.

16. The composition of any one of claims 1-15, wherein the protection molecule comprises a cytidine deaminase.

17. The composition of claim 16, wherein the cytidine deaminase is a human cytidine deaminase.

18. The composition of claim 16 or 17, wherein the cytidine deaminase comprises the amino acid sequence provided in Table 3.

19. The composition of any one of claims 1-18, wherein the cytotoxic agent comprises an anti-cancer agent.

20. The composition of any one of claims 1-19, wherein the cytotoxic agent comprises an alkylating agent, an antimetabolite, a nucleotide analog, a taxane, a platinum-based agent, and/or an antibiotic.

21. The composition of any one of claims 1-20, wherein the cytotoxic agent comprises gemcitabine.

22. The composition of any one of claims 1-21, wherein the antigen-binding protein and protection molecule are conjugated through click chemistry.

23. The composition of claim 22, wherein the click chemistry is copper-free click chemistry.

24. The composition of any one of claims 1-23, wherein the protection molecule is conjugated to a primary amine on the antigen-binding protein.

25. The composition of any one of claims 1-21, wherein the antigen-binding protein and protection molecule are conjugated as a recombinant protein.

26. The composition of any one of claims 1-21, wherein the antigen-binding protein and protection molecule are conjugated by cognate affinity-binding molecules.

27. The composition of 26, wherein the cognate affinity-binding molecules comprise biotin and streptavidin and/or biotin and avidin.

28. The composition of 26, wherein the cognate affinity-binding molecules comprise a SpyTag and SpyCatcher.

29. The composition of any one of claims 1-28, wherein the antigen-binding protein is cleavable from the protection molecule.

30. The composition of any one of claims 1-29, wherein the protection molecule is conjugated to the N-terminus and/or C-terminus of the antigen-binding protein.

31. The composition of any one of claims 1-30, wherein the composition also comprises the cytotoxic agent.

32. The composition of claim 31, wherein the composition comprises an amount of the cytotoxic agent greater than an amount within the cytotoxic agent's therapeutic window.

33. A protein comprising a heavy chain amino acid sequence provided in Table 1, a light chain amino acid sequence provided in Table 1, and a cytidine deaminase sequence provided in Table 3.

34. The protein of claim 33, further comprising a linker amino acid sequence provided in Table 2 and/or a His tag provided in Table 4.

35. A CD117-binding protein-cytidine deaminase conjugate comprising an amino acid sequence provided in Table 5.

36. A method of treating a patient that has, or will receive, a cytotoxic agent, the method comprising administering an antigen-binding protein conjugated to a protection molecule, wherein the protection molecule comprises a molecule capable of neutralizing the cytotoxic agent.

37. The method of claim 36, wherein the antigen-binding protein is an antibody or functional fragment thereof.

38. The method of claim 36 or 37, wherein the antigen-binding protein is capable of binding to an antigen expressed on a healthy cell.

39. The method of claim 38, wherein the healthy cell is a rapidly dividing healthy cell.

40. The method of claim 38 or 39, wherein the healthy cell is a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell.

41. The method of any one of claims 36-40, wherein the antigen-binding protein is capable of binding to CD34.

42. The method of any one of claims 36-40, wherein the antigen-binding protein is not capable of binding to CD34.

43. The method of any one of claims 36-40, wherein the antigen-binding protein is capable of binding to CD117.

44. The method of any one of claims 36-40, wherein the antigen-binding protein is not capable of binding to CD117.

45. The method of any one of claims 36-40, wherein the antigen-binding protein comprises a heavy chain amino acid sequence provided in Table 1 and a light chain amino acid sequence provided in Table 1, or a functional fragment thereof.

46. The method of any one of claims 36-45, wherein the antigen-binding protein and/or protein molecule comprises a purification tag.

47. The method of claim 46, wherein the purification tag comprises a 6×Histidine tag.

48. The method of claim 46 or 47, wherein the purification tag comprises a protease cleavage sequence.

49. The method of any one of claims 36-48, wherein the antigen-binding protein does not comprise an Fc region.

50. The method of any one of claims 36-49, wherein the protection molecule comprises an enzyme.

51. The method of any one of claims 36-50, wherein the protection molecule comprises a cytidine deaminase.

52. The method of claim 51, wherein the cytidine deaminase is a human cytidine deaminase.

53. The composition of claim 51 or 52, wherein the cytidine deaminase comprises the amino acid sequence provided in Table 3.

54. The method of any one of claims 36-53, wherein the cytotoxic agent comprises an anti-cancer agent.

55. The method of any one of claims 36-54, wherein the cytotoxic agent comprises an alkylating agent, an antimetabolite, a nucleotide analog, a taxane, a platinum-based agent, and/or an antibiotic.

56. The method of any one of claims 36-55, wherein the cytotoxic agent comprises gemcitabine.

57. The method of any one of claims 36-56, wherein the antigen-binding protein and protection molecule are conjugated through click chemistry.

58. The method of claim 57, wherein the click chemistry is copper-free click chemistry.

59. The method of any one of claims 36-58, wherein the protection molecule is conjugated to a primary amine on the antigen-binding protein.

60. The method of any one of claims 36-56, wherein the antigen-binding protein and protection molecule are conjugated as a recombinant protein.

61. The method of any one of claims 36-56, wherein the antigen-binding protein and protection molecule are conjugated by cognate affinity-binding molecules.

62. The method of 61, wherein the cognate affinity-binding molecules comprise biotin and streptavidin and/or biotin and avidin.

63. The method of 61, wherein the cognate affinity-binding molecules comprise a Spy Tag and SpyCatcher.

64. The method of any one of claims 36-63, wherein the antigen-binding protein is cleavable from the protection molecule.

65. The method of any one of claims 36-64, wherein the protection molecule is conjugated to the N-terminus and/or C-terminus of the antigen-binding protein.

66. The method of any one of claims 36-65, wherein the patient received, or will receive, an amount of the cytotoxic agent that is greater than an amount in the cytotoxic agent's therapeutic window.

67. The method of any one of claims 36-66, wherein the patient is administered the antigen-binding protein prior to receiving the cytotoxic agent.

68. The method of any one of claims 36-67, wherein the patient is administered the antigen-binding protein concurrently with or in the same composition as the cytotoxic agent.

69. The method of any one of claims 36-68, wherein the patient is administered the antigen-binding protein subsequent to receiving the cytotoxic agent.

70. The method of any one of claims 36-69, wherein the patient has, has been diagnosed with, has one or more symptoms of, or is suspected of having a disease indicated for the cytotoxic agent.

71. The method of any one of claims 36-70, wherein the patient has, has been diagnosed with, has one or more symptoms of, or is suspected of having a cancer, an infection, or an autoimmune disease.

72. The method of any one of claims 36-71, wherein the patient has had an adverse reaction to the cytotoxic agent.

73. The method of any one of claims 36-72, wherein the patient is indicated to receive the cytotoxic agent.

74. The method of any one of claims 36-73, wherein the antigen-binding protein is administered by injection.

75. The method of any one of claims 36-74, wherein the antigen-binding protein is administered intravenously.

76. A method of reducing one or more side effects of a cytotoxic agent, the method comprising administering to a patient an antigen-binding protein conjugated to a protection molecule, wherein the protection molecule comprises a molecule capable of neutralizing the cytotoxic agent.

77. The method of claim 76, wherein the antigen-binding protein is an antibody or functional fragment thereof.

78. The method of claim 76 or 77, wherein the antigen-binding protein is capable of binding to an antigen expressed on a healthy cell.

79. The method of claim 78, wherein the healthy cell is a rapidly dividing healthy cell.

80. The method of claim 78 or 79, wherein the healthy cell is a gastrointestinal cell, a bone marrow cell, an endothelial cell, a progenitor cell, a dermal cell, a cardiac cell, a liver cell, a kidney cell, a lung cell, an immune cell, a nervous system cell, and/or a mucosal cell.

81. The method of any one of claims 76-80, wherein the antigen-binding protein is capable of binding to CD34.

82. The method of any one of claims 76-80, wherein the antigen-binding protein is not capable of binding to CD34.

83. The method of any one of claims 76-80, wherein the antigen-binding protein is capable of binding to CD117.

84. The method of any one of claims 76-80, wherein the antigen-binding protein is not capable of binding to CD117.

85. The method of any one of claims 76-80, wherein the antigen-binding protein comprises a heavy chain amino acid sequence provided in Table 1 and a light chain amino acid sequence provided in Table 1, or a functional fragment thereof.

86. The method of any one of claims 76-85, wherein the antigen-binding protein comprises a purification tag.

87. The method of claim 86, wherein the purification tag comprises a 6×Histidine tag.

88. The method of claim 86 or 87, wherein the purification tag comprises a protease cleavage sequence.

89. The method of any one of claims 76-88, wherein the antigen-binding protein does not comprise an Fc region.

90. The method of any one of claims 76-89, wherein protection molecule comprises an enzyme.

91. The method of any one of claims 76-90, wherein the protection molecule comprises a cytidine deaminase.

92. The method of claim 91, wherein the cytidine deaminase is a human cytidine deaminase.

93. The composition of claim 91 or 92, wherein the cytidine deaminase comprises the amino acid sequence provided in Table 3.

94. The method of any one of claims 76-93, wherein the cytotoxic agent comprises an anti-cancer agent.

95. The method of any one of claims 76-94, wherein the cytotoxic agent comprises an alkylating agent, an antimetabolite, a nucleotide analog, a taxane, a platinum-based agent, and/or an antibiotic.

96. The method of any one of claims 76-95, wherein the cytotoxic agent comprises gemcitabine.

97. The method of any one of claims 76-96, wherein the antigen-binding protein and protection molecule are conjugated through click chemistry.

98. The method of claim 97, wherein the click chemistry is copper-free click chemistry.

99. The method of any one of claims 76-98, wherein the protection molecule is conjugated to a primary amine on the antigen-binding protein.

100. The method of any one of claims 76-96, wherein the antigen-binding protein and protection molecule are conjugated as a recombinant protein.

101. The method of any one of claims 76-96, wherein the antigen-binding protein and protection molecule are conjugated by cognate affinity-binding molecules.

102. The method of 101, wherein the cognate affinity-binding molecules comprise biotin and streptavidin and/or biotin and avidin.

103. The method of 101, wherein the cognate affinity-binding molecules comprise a Spy Tag and SpyCatcher.

104. The method of any one of claims 76-103, wherein the antigen-binding protein is cleavable from the protection molecule.

105. The method of any one of claims 76-104, wherein the protection molecule is conjugated to the N-terminus and/or C-terminus of the antigen-binding protein.

106. The method of any one of claims 76-105, wherein the patient received, or will receive, an amount of the cytotoxic agent that is greater than an amount in the cytotoxic agent's therapeutic window.

107. The method of any one of claims 76-106, wherein the patient is administered the antigen-binding protein prior to receiving the cytotoxic agent.

108. The method of any one of claims 76-107, wherein the patient is administered the antigen-binding protein concurrently with or in the same composition as the cytotoxic agent.

109. The method of any one of claims 76-108, wherein the patient is administered the antigen-binding protein subsequent to receiving the cytotoxic agent.

110. The method of any one of claims 76-109, wherein the patient has, has been diagnosed with, has one or more symptoms of, or is suspected of having a disease indicated for the cytotoxic agent.

111. The method of any one of claims 76-110, wherein the patient has, has been diagnosed with, has one or more symptoms of, or is suspected of having a cancer, an infection, or an autoimmune disease.

112. The method of any one of claims 76-111, wherein the patient has had an adverse reaction to the cytotoxic agent.

113. The method of any one of claims 76-112, wherein the patient is indicate to receive the cytotoxic agent.

114. The method of any one of claims 76-113, wherein the antigen-binding protein is administered by injection.

115. The method of any one of claims 76-114, wherein the antigen-binding protein is administered intravenously.

116. The method of any one of claims 76-115, wherein the side effects comprise neutropenia, anemia, thrombocytopenia, lymphopenia, or a combination thereof.

117. The method of any one of claims 76-116, wherein the side effects occur or are worsened when the patient receives the cytotoxic agent at a dosage above the therapeutic window of the cytotoxic agent.
